(19) United States
(12) Patent Application Publication
(10) Pub. No.: US 2023/0405122 A1
(43) Pub. Date: Dec. 21, 2023

(54) COMPOSITIONS AND USES OF PSCA TARGETED CHIMERIC ANTIGEN RECEPTOR MODIFIED CELLS

(71) Applicant: City of Hope, Duarte, CA (US)

(72) Inventors: Jianhua Yu, Wilmington, DE (US); Michael A. Caligiuri, Wilmington, DE (US)

(21) Appl. No.: 18/038,423

(22) PCT Filed: Nov. 23, 2021
(86) PCT No.: PCT/US2021/060500

§ 371 Date: May 23, 2023

Related U.S. Application Data
(60) Provisional application No. 63/117,904, filed on Nov. 24, 2020.
(60) Provisional application No. 63/172,489, filed on Apr. 8, 2021.

(51) Int. Cl. A61K 39/00; C07K 14/54; C07K 16/30; C07K 14/705; C07K 14/725; A61P 35/00; C12N 5/0783

(52) U.S. Cl. CPC A61K 39/464493; C07K 14/5443; C07K 16/3069; A61K 39/4631; A61K 39/4613; C07K 14/70521; C07K 14/7051; A61P 35/00; C12N 5/0646; C07K 2317/622; C07K 2319/03; A61K 2239/54; A61K 2239/26

(57) ABSTRACT

Nucleic acid molecules encoding an IL-15 domain and a chimeric antigen receptor (CAR) that targets cells expressing prostate stem cell antigen are provided as well as polypeptides encoded thereby. Vectors and host cells such as immune cells containing the nucleic acid molecules also are disclosed, as well as methods for their use.

### CROSS-REFERENCE TO RELATED APPLICATIONS

This application claims priority to U.S. Provisional Application No. 63/117,904, filed Nov. 24, 2020 and U.S. Provisional Application No. 63/172,489, filed Apr. 8, 2021, the contents of each are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 9, 2021, is named 113086-0230_SL.txt and is 90,793 bytes in size.

### TECHNICAL FIELD

This disclosure concerns PSCA-specific chimeric antigen receptor (CAR)-engineered immune cells, methods of formulating, and methods of use.

### BACKGROUND

Prostate stem cell antigen (PSCA) is highly expressed in various solid tumor cells, including pancreatic cancer, prostate cancer, and urinary bladder cancer but has limited expression in normal cells (Argani et al., (2001) Cancer Res. 61, 4320-4324; Abate-Daga et al., (2014) Hum Gene Ther. 25(12), 1003-1012). Pancreatic cancer remains the 4th leading cause of cancer-related deaths in the United States despite being the 10th most frequently diagnosed malignancy (Siegel et al., (2012) CA Cancer J. Clin. 62, 10-29). Most patients present with locally advanced or metastatic disease at diagnosis and are therefore not eligible for surgical resection. In addition, pancreatic cancer cells tend to be intrinsically resistant to chemo- and radiotherapy. The standard of care is gemcitabine-based chemotherapy, which reduces morbidity but does not induce a proven survival benefit. Median survival is currently estimated to be 6-8 months (Cartwright et al., (2008) Cancer Control 15, 308-313). More effective therapies are needed to effectively treat these PSCA-positive cancers.

### SUMMARY OF THE DISCLOSURE

Provided herein is a nucleic acid molecule comprising, or alternatively consisting essentially of, or yet further consisting of a first nucleotide molecule encoding a chimeric antigen receptor (CAR) and a second nucleotide molecule encoding an IL-15 domain. The CAR comprises, or alternatively consists essentially of, or yet further consists of a single chain variable fragment (scFv) targeting prostate stem cell antigen (PSCA), a spacer, a transmembrane domain, a co-stimulatory domain and a CD3 ξ signaling domain. The nucleic acid molecule can be DNA or RNA.

Described herein are methods for making and using CAR T and natural killer (NK) cells or other immune cells expressing a PSCA targeted chimeric antigen receptor (CAR) (also herein called PSCA CAR NK cells) co-expressing an IL-15 domain (e.g., at least a portion of IL-15, at least a portion of IL-15Ra, or a fusion protein that includes at least a portion of IL-15 and at least a portion of IL-15Ra) to treat a variety of solid tumors, (e.g., non-small cell lung carcinoma, gall bladder cancer, pancreatic cancer, prostate cancer, and urinary bladder cancer). The PSCA CAR NK cells described herein possess potent antigen-specific anti-tumor efficacy in vitro and in vivo. The PSCA CAR NK cells described herein also possess the potent antigen-specific anti-tumor efficacy.

Described herein is a nucleic acid molecule comprising a nucleotide sequence encoding a chimeric antigen receptor (CAR) or a polypeptide, wherein the chimeric antigen receptor or polypeptide comprises: an scFv targeting PSCA, a spacer, a transmembrane domain, a co-stimulatory domain, and a CD3 ξ signaling domain, and a nucleotide sequence encoding a polypeptide comprising an IL-15 domain.

In some embodiments, the CAR encoded by the first nucleotide molecule comprises or alternatively consists essentially of, or yet further consists of an scFv comprising or alternatively consisting essentially of, or yet further consisting of a heavy chain (HC) complementary-determining region (CDR) 1 (CDRH1) comprising DYYI (aa 31 to aa 34 of SEQ ID NO: 33), an HC CDR 2 (CDRH2) comprising WIDPENGDTEFVPKFQG (aa 50 to aa 66 of SEQ ID NO: 33), and an HC CDR 3 (CDRH3) comprising GGF (aa 99 to aa 101 of SEQ ID NO: 33). In some embodiments, the CAR encoded by the first nucleotide sequence comprises or alternatively consists essentially of, or yet further consists of an scFv comprising or alternatively consisting essentially of, or yet further consisting of a light chain (LC) complementarity-determining region (CDR) 1 (CDRL1) comprising SASSSVRFIH (aa 24 to aa 33 of SEQ ID NO: 32), an LC CDR 2 (CDRL2) comprising DTSKLAS (aa 49 to aa 55 of SEQ ID NO: 32), and an LC CDR 3 (CDRL3) comprising QQWGSSPFT (aa 88 to aa 96 of SEQ ID NO: 32).

In another aspect, the nucleic acid molecule encoding the IL-15 domain comprises a soluble IL-15 (sIL-15 or s15), a membrane bound IL-15 (mbIL-15 or mIL-15 or m15), a fusion protein that includes soluble IL-15 and at least a portion of IL-15Rα (sIL-15c or s15c), and a fusion protein that includes a transmembrane domain, at least a portion of IL-15 and at least a portion of IL-15a (mbIL-15c or mIL-15c or m15c) that can optionally be codon-optimized.

Also provided is a vector comprising, or alternatively consisting essentially of, or yet further consisting of a nucleic acid molecule as disclosed herein. In some embodiments, the vector further comprises a regulatory sequence operatively linked to one or more elements of the nucleic acid molecule that direct the expression or the replication of the nucleic acid molecule.

Also described is an expression vector comprising a nucleic acid molecule described herein and a population of human immune cells (e.g., NK cells, T cells or macrophages) transduced by the vector or harboring a nucleic acid molecule (e.g., mRNA) descried herein.

Also described is a method of treating a solid tumor or cancer in a patient comprising administering a population of autologous or allogeneic human CAR cells or human NK cells transduced by a vector comprising the nucleic acid molecule described herein, wherein the solid tumor or cancer comprises cells expressing PSCA. In various embodiments: the population of human CAR or NK cells expressing the chimeric antigen receptor or the polypeptide is administered locally or systemically; the population of human CAR or NK cells expressing the chimeric antigen receptor or the polypeptide is administered by single or repeat dosing.

In various embodiments, the NK cells are derived from cord blood, peripheral blood or stem cells.

Further provided herein are a CAR and IL-15 domain polypeptides encoded by the disclosed nucleic acid molecules as well as cells expressing and comprising the same.

Additionally provided is an isolated cell comprising one or more of: polypeptides as disclosed herein, a nucleic acid molecule as disclosed herein, or a vector as disclosed herein. In some embodiments, the cell expresses a Chimeric Antigen Receptor (CAR) and IL-15 domain as disclosed herein. In some embodiments the cell is an immune cell and optionally a T-cell, B-cell, an NK cell, an NKT cell, a dendritic cell, a myeloid cell, a monocyte, or a macrophage.

Additionally provided herein is a population of cells comprising, or alternatively consisting essentially of, or yet further consisting of the isolated cell disclosed herein. In some embodiments provided herein is a population of immune cells, e.g., human NK cells, T cells, or macrophages comprising or alternatively consisting essentially of, or yet further consisting of the nucleic acid molecule or the CAR and IL-15 domain encoded by the nucleic acid molecule disclosed herein.

Further provided is a human NK cells transduced by a vector comprising a nucleic acid of this disclosure, e.g., encoding the CAR, wherein the human NK cells are stable after one, two, or three freeze-thaw cycles. They can be used in the methods of this disclosure, and the cells are frozen prior to administration and optionally wherein the cells are NK cells. In another aspect, the cells are thawed before administration and optionally wherein the cell are NK cells.

Additionally, provided herein is a composition comprising, or alternatively consisting essentially of, or yet further consisting of a carrier and one or more of a nucleic acid molecule, a vector, a polypeptide, a cell or population of cells disclosed herein.

In one aspect, provided is a method for producing a cell line of a cell disclosed herein comprising or alternatively consisting essentially of, or yet further consisting of introducing a nucleic acid disclosed herein or a vector disclosed herein to a cell disclosed herein, and expressing the nucleic acid molecule.

In yet another aspect, provided is a method of inhibiting the growth of a cancer cell expressing PSCA or a tissue comprising the cancer cell, the method comprising, or alternatively consisting essentially of, or yet further consisting of contacting the cancer cell or the tissue with a cell disclosed herein.

In yet another aspect, provided is a method of treating a cancer that expresses PSCA in a subject in need thereof, comprising, or alternatively consisting essentially of, or yet further consisting of administering to the subject a cell or a population of cells disclosed herein, thereby treating the cancer.

In yet another aspect, provided is a method for treating a solid tumor or cancer in a patient in need thereof, comprising, or alternatively consisting essentially of, or yet further consisting of administering a population of autologous or allogenic human NK cells transduced by a vector comprising, or alternatively consisting essentially of, or yet further consisting of a nucleic acid molecule disclosed herein, and wherein the solid tumor or cancer comprises cells expressing PSCA.

In some embodiments, described herein is a method of treating a solid tumor or a method of reducing or eliminating PSCA-positive cells. In some embodiments, solid tumor is any one or more of a pancreatic cancer, prostate cancer, bladder cancer, gastric cancer, breast cancer, cervical cancer, endometrial cancer, esophageal cancer, lung cancer, ovarian cancer, testicular cancer, thyroid cancer, etc. or a subpopulation of these or other cancers Also described herein is a method of treating PSCA-positive cancers or disorders (including, e.g., pancreatic cancer, prostate cancer, and urinary bladder cancer) in a patient comprising administering a population of autologous or allogeneic human NK cells transduced by a vector comprising a nucleic acid molecule described herein, wherein the PSCA-positive cancers or disorders comprise cells expressing PSCA. In various embodiments: human NK cells expressing a chimeric antigen receptor or polypeptide described herein are administered locally or systemically; the PSCA-expressing target cells are cancerous cells; and the human NK cells expressing chimeric antigen receptor or polypeptide are administered by single or repeat dosing.

Also described herein are methods for using PSCA CAR NK cells as anti-cancer agents selective against PSCA-positive cell; also described herein are methods of decreasing the population of non-cancerous PSCA-positive cells. In some embodiments, described herein is a method of reducing or eliminating PSCA-positive cells in a subject comprising administering a population of autologous or allogeneic human NK cells transduced by a vector comprising the nucleic acid molecule encoding a PSCA CAR or a PSCA polypeptide.

In yet another aspect, provided is a method of reducing or eliminating PSCA-positive cells in a subject comprising administering a population of autologous or allogeneic human NK cells transduced by a vector comprising a nucleic acid molecule disclosed herein.

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a NK cell, and most preferably an autologous NK cell.

The CAR or polypeptide can be transiently expressed in a NK cell population by an mRNA encoding the CAR or polypeptide. The mRNA can be introduced into the NK cells by electroporation (Wiesinger et al. 2019 Cancers (Basel) 11:1198).

In some embodiments, described herein is a method of increasing survival of a subject having cancer comprising administering a composition comprising a CAR NK cell described herein.

In some embodiments, described herein is a method of treating a cancer in a patient comprising administering a composition comprising a CAR NK cell described herein.

In some embodiments, described herein is a method of reducing or ameliorating a symptom associated with a cancer in a patient comprising administering a composition comprising a CAR NK cell described herein.

In some embodiments, a composition comprising CAR NK cells or CAR T cells described herein is administered locally or systemically. In some embodiments, a composition comprising CAR NK cells described herein is administered by single or repeat dosing. In some embodiments, a composition comprising CAR NK cells described herein is administered to a patient having a cancer, a pathogen infection, an autoimmune disorder, or undergoing allogeneic transplant.

In some embodiments, the cancer is a solid tumor. In some embodiments, the cancer is selected from the group consisting of pancreatic cancer, prostate cancer, and urinary bladder. In some embodiments, the cancer is any cancer or tumor that comprises a PSCA-positive cell.

Additionally, provided herein is a kit comprising, or alternatively consisting essentially of, or yet further consisting of optional instructions for use and one or more of a nucleic acid molecule, vector, polypeptide, cell, population of cells, or composition disclosed herein.

### DESCRIPTION OF DRAWINGS

FIG. 1 shows a graph depicting PSCA antigen RNA expression in various tumor tissues. The dotted line represents normal PSCA level, and the box outlines PSCA expression level of pancreatic cancer.

FIG. 2 shows a schematic depicting various exemplary CAR constructs. SP: signal peptide; TM: transmembrane; Codon Opm IL15: codon optimized IL15, either soluble or membrane bound.

FIGS. 3A-3B shows in vitro cell lysis as a result of treatment with PSCA-CAR NK cells bearing the CD28 or 2B4 signaling domain compared to NK cells expressing truncated CD19 without the PSCA-CAR and untreated. FIG. 3A shows PSCA-CAR NK cells lyse PSCA(+) Canpan-1 lyse pancreatic tumor cells in an antigen-specific manner. FIG. 3B shows there is no specific CAR activity of PSCA-CAR NK cells against PSCA(−) PANC-1 cell line.

FIGS. 4A-4B show percent cell lysis at various Effector: Target (E:T) ratios of two PSCA-CAR with A1 or M1 scFv in engineered NK cells compared to no treatment. FIG. 4A shows PSCA-CAR NK cells lyse PSCA(+) Canpan-1 lyse pancreatic tumor cells in an antigen-specific manner. FIG. 4B shows there cell lysis of PSCA-CAR NK cells against PSCA(−) PANC-1 cell line. Data were collected from ⁵¹Cr release assay.

FIGS. 5A-5B show cytotoxicity of PSCA CAR NK cells with 4 different types of IL-15 against PSCA(+) Canpan-1 pancreatic cancer cells (FIG. 5A) and PSCA(−) PANC-1 cell line (FIG. 5B) in series of Effector: Target (E:T) ratios. Cytotoxic effect was analyzed from a ⁵¹Cr release assay.

FIG. 6 shows cytotoxicity of PSCA-CAR NK cells with 4 different types of IL-15 against PSCA(+) Canpan-1 pancreatic cancer cells. The top panel shows normalized cell index data obtained from the xCELLigence Real-Time Cell Analysis (RTCA), indicating the tumor growth index overtime against CAPAN-1 cells. The bottom panel shows quantitative data at one time point of around 25 hours (see the vertical solid line in the top panel) after the co-culture of tumor cells and CAR NK cells was initiated. The legends used indicate the treatment for each group for both the top and the bottom panel. From left to right: tEGFR, PSCA CAR with M1 scFv, A1-t19, A1-m15c, A1 s15c, A1-m15, A1-s15, and “tumor only” untreated.

FIGS. 7A-7C show tumor depletion effect of PSCA NK cells in pancreatic cancer engrafted mouse model. FIG. 7A shows IVIS imaging of pancreatic cancer (CAPAN1_luc, expressing a luciferase gene) engrafted mice. Mice were imaged prior- and post-NK treatment. Frozen PSCA-CAR-sIL15 NK cells were thawed and injected directly to the mice systematically (i.v.) and locally (i.p.). FIG. 7B shows quantification of bioluminescence of pancreatic cancer cells. FIG. 7C shows quantification of bioluminescence of pancreatic cancer cells. Quantification was achieved by normalizing to the pre-treatment and presented as fold changes.

FIGS. 8A-8B show the in vitro efficacy of PSCA CAR NK cells-expressing soluble IL-15. PSCA CAR NK cells-expressing soluble IL-15 were tested for cytotoxicity against two different pancreatic cell lines. FIG. 8A shows the impact on Capan-1 (PSCA+) cells and FIG. 8B shows the impact on Panc-1 (PSCA−) cells. Cytotoxicity was tested after coculture of NK cells with pancreatic cells for a duration of 72 hrs at an E:T ratio of 1:3.

FIG. 9 shows functional comparison of research and clinical construct by RTCA assay. Research and clinical PSCA CAR NK vector expressing soluble IL-15 were tested for cytotoxicity against Capan-1 (PSCA+) cells. Non-human DNA sequences were removed from the research grade PSCA CAR—soluble IL-15 vector to create the clinical grade PSCA CAR—soluble IL-15 vector. Cytotoxicity was tested after co-culture of indicated NK cells or PSCA CAR—soluble NK cells with pancreatic cells for a duration of 72 hrs at an E:T ratio of 1:3. Cell growth was analyzed by the xCELLigence Real-Time Cell Analysis (RTCA).

FIGS. 10A-10D show the anti-tumor effect of PSCA NK cells. FIG. 10A is a schematic depiction of the study. NSG mice were intraperitoneally engrafted with 0.2×10⁹ CAPAN1 cells expressing luciferase (luc) then received three cycles of treatment with NK cells expressing soluble IL-15 or PSCA CAR and soluble IL-15. Each cycle included four injections each composed of 2×10⁹ cells administered intraperitoneally and 2×10⁹ administered intravenously. FIG. 10B is IVIS imaging of mice that were engrafted with CAPAN1_luc and treated with or without the indicated NK or CAR NK cells expressing soluble IL-15. Mice were imaged prior- and post-NK treatment. FIG. 10C shows a quantification of bioluminescence of pancreatic cancer cells. FIG. 10D shows survival analysis of mice engrafted with CAPAN1_luc and treated with or without indicated NK cells or CAR NK cells expressing soluble IL-15.

FIGS. 11A-11B show the cell killing effect of a combination of PSCA CAR—soluble IL-15 NK cells and aldoxorubicin. FIG. 11A is a series of representative images of 48 hr post co-culture of the pancreatic cancer cell line CAPAN1 with PSCA CAR—soluble IL-15 NK cells (Effector/Target=1:3, PSCA CAR NK) or aldoxorubicin (0.3 μM) or in combination. FIG. 11B depicts an analysis of normalized tumor growth index of CAPAN1 treated with PSCA CAR—soluble IL-15 NK cells, aldoxorubicin (0.3 μM), or in combination, analyzed by the xCELLigence Real-Time Cell Analysis (RTCA). NK cells expressing soluble IL-15 served as control.

FIGS. 12A-12B show the cell killing effect of a combination of PSCA CAR—soluble IL-15 NK cells and gemcibabine. FIG. 12A is a series of representative images of 48 hr post co-culture of the pancreatic cancer cell line CAPAN1 with PSCA CAR NK—soluble IL-15 cells expressing (Effector/Target=1:3, PSCA CAR NK) or gemcibabine (0.3 μM) or in combination. FIG. 12B depicts an analysis of normalized tumor growth index of CAPAN1 treated with PSCA CAR—soluble IL-15 NK cells, aldoxorubicin (0.3 μM), or in combination, analyzed by the xCELLigence Real-Time Cell Analysis (RTCA). NK cells expressing soluble IL-15 served as control.

FIGS. 13A-13B show Capan-1 tumor cell killing by freshly prepared (FIG. 13A) and previously frozen (FIG. 13B) NK cells.

FIGS. 14A-14D show the anti-tumor effect of PSCA NK cells. FIG. 14A is IVIS imaging of mice that were engrafted with CAPAN1_luc and treated with or without the indicated NK expressing soluble IL-15 or PSCA CAR NK cells expressing soluble IL-15. Mice were imaged prior- and post-NK treatment. FIG. 14B shows imaging the pancreas under three different treatments. FIG. 14C shows survival analysis of mice engrafted with CAPAN1_luc and treated with or without indicated NK cells expressing soluble IL-15 or PSCA CAR NK cells expressing soluble IL-15. FIG. 14D shows quantification of tumor cells and NK cells under three different treatments.

### DETAILED DESCRIPTION

In this disclosure, the generation of and anti-tumor efficacy of CAR with an anti-PSCA antigen-binding domain are described, inter alia.

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

### Definitions

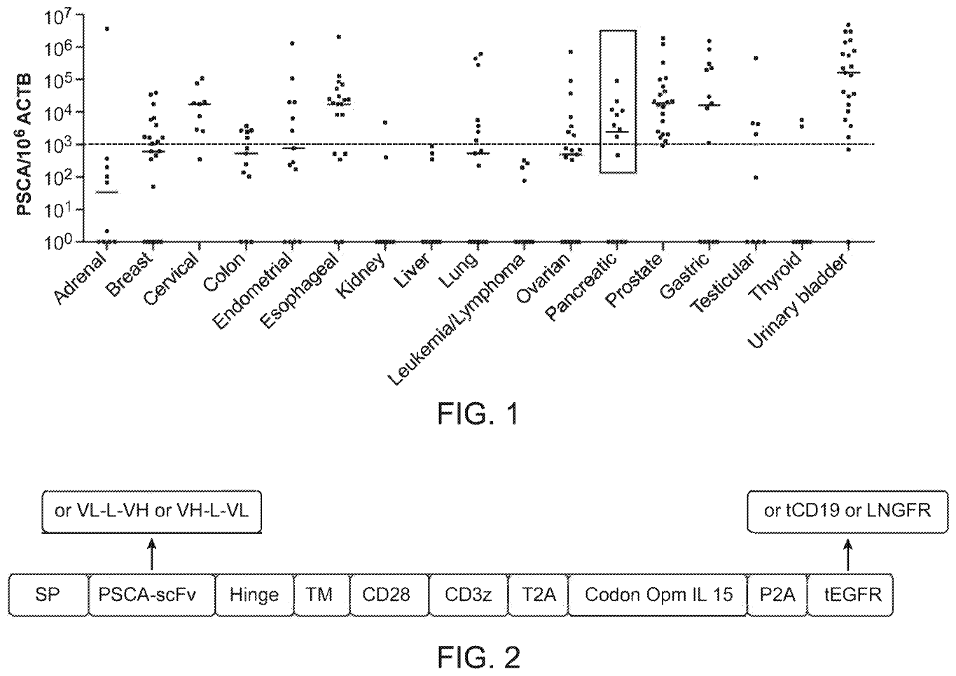

As used herein, Prostate Stem Cell Antigen or PSCA, refers to a glycosylphosphatidylinositol-anchored cell membrane glycoprotein that is highly expressed in the prostate. The protein is also expressed in the tissues identified in FIG. 1, e.g., bladder, placenta, colon, kidney, and stomach. PSCA is up-regulated in a large proportion of prostate cancers. The gene encoding for PSCA includes a polymorphism that results in an upstream start codon in some individuals; this polymorphism is thought to be associated with a risk for certain gastric and bladder cancers. Diseases associated with PSCA include prostate cancer, gallbladder adenocarcinoma and transitional cell carcinoma. Among its related pathways are metabolism of proteins and post-translational modification-synthesis of GPI-anchored proteins.

The term “about,” as used herein when referring to a measurable value such as an amount or concentration and the like, is meant to encompass variations of 20%, 10%, 5%, 1%, 0.5%, or even 0.1% of the specified amount.

As used in the specification and claims, the singular form “a”, “an”, and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human. In some embodiments, they refer to and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human.

Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

As used herein, the term “animal” refers to living multi-cellular vertebrate organisms, a category that includes, for example, mammals and birds. The term “mammal” includes both human and non-human mammals.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” or “isolated nucleic acid molecule” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “polynucleotide”, “nucleic acid sequence”, “nucleic acid molecule”, and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. In some embodiments, the polynucleotide as disclosed herein is a RNA. In some embodiments, the polynucleotide as disclosed herein is a DNA. In some embodiments, the polynucleotide as disclosed herein is a hybrid of DNA and RNA.A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample.

In one aspect, the expression level of a gene from one sample may be directly compared to the expression level of that gene from a control or reference sample. In another aspect, the expression level of a gene from one sample may be directly compared to the expression level of that gene from the same sample following administration of a compound.

A “gene product” or alternatively a “gene expression product” refers to the amino acid (e.g., peptide or polypeptide) generated when a gene is transcribed and translated.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

In some embodiments, the terms “first” “second” “third” “fourth” or similar in a component name are used to distinguish and identify more than one components sharing certain identity in their names. For example, “first nucleotide molecule” and “second nucleotide molecule” may be used in the specification or claims to distinguish two nucleotide sequences or molecules, and in some embodiments, the first nucleotide molecule may refer to a nucleotide molecule that encode for a chimeric antigen receptor (CAR) as disclosed herein while the second nucleotide molecule may refer to a nucleotide molecule that encodes for an IL-15 domain.

The term “a regulatory sequence or molecule” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EF1alpha promoter and the CMV promoter. The EF1alpha sequence is known in the art (see, e.g., addgene.org/11154/sequences/; ncbi.nlm.nih.gov/nuccore/J04617, each last accessed on Mar. 13, 2019, and Zheng and Baum (2014) Int'l. J. Med. Sci. 11(5):404-408). The CMV promoter sequence is known in the art (see, e.g., snapgene.com/resources/plasmid-files/?set=basic_cloning_vectors&plasmid=CMV_promoter, last accessed on Mar. 13, 2019 and Zheng and Baum (2014), supra.).

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “enhancer” as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST. In another embodiment, the program is any one of: Clustal Omega accessible at www.ebi.ac.uk/Tools/msa/clustalo/, Needle (EMBOSS) accessible at www.ebi.ac.uk/Tools/psa/emboss_needle/, Stretcher (EMBOSS) accessible at www.ebi.ac.uk/Tools/psa/emboss_stretcher/, Water (EMBOSS) accessible at www.ebi.ac.uk/Tools/psa/emboss_water/, Matcher (EMBOSS) accessible at www.ebi.ac.uk/Tools/psa/emboss_matcher/, LALIGN accessible at www.ebi.ac.uk/Tools/psa/lalign/. In further embodiments, the default setting is used.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complement. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” as it is applied to the production of cells, such as chimeric antigen receptor cells, refers to the process whereby a foreign nucleotide sequence is introduced into a cell. In some embodiments, this transduction is done via a vector, viral or non-viral.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014. In some embodiments, the term “antibody” refers to a single-chain variable fragment (scFv, or ScFV). In some embodiments, the term “antibody” refers to more than one single-chain variable fragments (scFv, or ScFV) linked with each other, optionally via a peptide linker or another suitable component as disclosed herein. In some embodiments, an antibody is a monoclonal antibody. In some embodiments, an antibody is a monospecific antibody or a multispecific antibody, such as a bispecific antibody or a trispecific antibody. The species of the antibody can be a human or non-human, e.g., mammalian.

As used herein, the language of “the proximity” of an immune cell, such as a T cell or an NK cell, refers to any location around the immune cell, a target of the immune cell (such as a cancer cell) present in which location can be recognized by the immune cell, such as via binding of a molecule present on the immune cell to another molecule present on the target, and optionally damaged or killed by the immune cell. Therefore, the action of bringing to the proximity of an immune cell is also referred to herein as engaging an immune cell.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDHR and light chain regions labeled CDLR). Thus, a CDHR3 is the CDR3 from the variable domain of the heavy chain of the antibody in which it is found, whereas a CDLR1 is the CDR1 from the variable domain of the light chain of the antibody in which it is found. For example, a PSCA antibody will have a specific V_{H} region and the V_{L} region sequence unique to the PSCA relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). In one aspect, the term “equivalent” or “biological equivalent” of an antibody or antigen binding fragment means the ability of the antibody or fragment to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

As used herein, the term “antigen binding domain” refers to any protein or polypeptide domain such as an antibody fragment that can specifically bind to an antigen target. Non-limiting examples of antibody fragments include a single-chain variable fragment (scFV or ScFV), a Fab, F(ab′)₂, Fab′, or Fv.

As used herein, a single-chain variable fragment, also referred to herein as a fragment of an antibody, and is a fusion protein of the variable regions of the heavy (V_{H}) and light chains (V_{L}) of immunoglobulins, optionally connected with a short linker peptide of about 10 to about 25 amino acids. The linker is usually rich in glycine for flexibility, as well as serine or threonine for solubility, and can either connect the N-terminus of the V_{H} with the C-terminus of the VL, or vice versa. This protein retains the specificity of the original immunoglobulin, despite removal of the constant regions and the introduction of the linker.

As used herein, a fragment crystallizable (Fc) region refers to the tail region of an antibody that stabilizes the antibody, and optionally interacts with (such as binds) an Fc receptor on an immune cell or on a platelet or that binds a complement protein.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement. In some embodiments, the term “equivalent” or “biological equivalent” of an antibody means the ability of the antibody to selectively bind its epitope protein or a fragment thereof as measured by ELISA or other suitable methods is substantively maintained, for example, at a level of at least 50%, or at least 55%, or at least 60%, or at least 65%, or at least 70%, or at least 75%, or at least 80%, or at least 85%, or at least 90%, or at least 95%, or at least 99%, or more. Biologically equivalent antibodies include, but are not limited to, those antibodies, peptides, antibody fragments, antibody variant, antibody derivative and antibody mimetics that bind to the same epitope as the reference antibody. Additionally or alternatively, the equivalent and the reference antibody shares the same set of CDRs but other amino acids are modified.

The polypeptide or an equivalent thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus (C-terminus). Additionally or alternatively, the polypeptide or an equivalent thereof can further comprises an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the amine-terminus (N-terminus).

An equivalent of a reference polypeptide comprises, consists essentially of, or alternatively consists of an polypeptide having at least 80% amino acid identity to the reference polypeptide, such as the CAR as disclosed herein, or a polypeptide that is encoded by a polynucleotide that hybridizes under conditions of high stringency to the complement of a polynucleotide encoding the reference polypeptide, such as a CAR or IL-15 domain as disclosed herein, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

By “fragment” is intended a molecule consisting of only a part of the intact full-length sequence and structure. The fragment of a polypeptide can include a C-terminal deletion, an N-terminal deletion, an internal deletion of the native polypeptide, or any combination thereof. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question substantially retains biological activity.

The term “antibody variant” intends to include antibodies produced in a species other than a mouse. It also includes antibodies containing post-translational modifications to the linear polypeptide sequence of the antibody or a fragment thereof. It further encompasses fully human antibodies.

As used herein, the term “specific binding” or “binding” means the contact between an antibody and an antigen with a binding affinity of at least 10⁻⁶ M. In certain embodiments, antibodies bind with affinities of at least about 10⁻⁷ M, and preferably at least about 10⁻⁸ M, at least about 10⁻⁹ M, at least about 10⁻¹⁰ M, at least about 10⁻¹¹ M, or at least about 10⁻¹² M.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

CD19 is a molecule that functions as co-receptor for the B-cell antigen receptor complex (BCR) on B-lymphocytes. It decreases the threshold for activation of downstream signaling pathways and for triggering B-cell responses to antigens, and is required for normal B cell differentiation and proliferation in response to antigen challenges. See, for example, de Rie et al., Cell Immunol. 1989 February; 118(2):368-81; and Carter and Fearon. Science. 1992 Apr. 3; 256(5053):105-7. The majority of B cell malignancies, such as Non-Hodgkin's Lymphoma (NHL), acute lymphoblastic leukemia (ALL), and chronic lymphocytic leukemia (CLL), express normal to high levels of CD19. In some embodiments, the CD19 is a human CD19. Non-limiting exemplary sequences of this protein or the underlying gene can be found under Gene Cards ID: GC16P033267, HGNC: 1633, NCBI Entrez Gene: 930, Ensembl: ENSG00000177455, OMIM®: 107265, or UniProtKB/Swiss-Prot: P15391, each of which is incorporated by reference herein in its entirety.

In some embodiments, CD19 refers to CD19 isoform 1 or CD19 isoform 2 or both. In further embodiments, CD19 isoform 1 comprises, or consists essentially of, or yet further consists of

MPPPRLLFFLLFLTPMEVRPEEPLVVKVEEGDNAVLQCLKGTSDGPTQQLTWSRESPLKPF LKLSLGLPGLGIIHMRPLAIWLFIFNVSQQMGGFYLCQPGPPSEKAWQPGWTVNVEGSGELF RWNVSDLGGLGCGLKNRSSEGPSSPSGKLMSPKLYVWAKDRPEIWEGEPPCLPPRDSLNQ SLSQDLTMAPGSTLWLSCGVPPDSVSRGPLSWTHVHPKGPKSLLSLELKDDRPARDMWV METGLLLPRATAQDAGKYYCHRGNLTMSFHLEITARPVLWHWLLRTGGWKVSAVTLAY LIFCLCSLVGILHLQRALVLRRKRKRMTDPTRRFFKVTPPPGSGPQNQYGNVLSLPTPTSGL GRAQRWAAGLGGTAPSYGNPSSDVQADGALGSRSPPGVGPEEEEGEGYEEPDSEEDSEFY ENDSNLGQDQLSQDGSGYENPEDEPLGPEDEDSFSNAESYENEDEELTQPVARTMDFLSPH GSAWDPSREATSLGSQSYEDMRGILYAAPQLRSIRGQPGPNHEEDADSYENMDNPDGPDP AWGGGGRMGTWSTR (SEQ ID NO:82), or aa 16 to aa 556 of SEQ ID NO: 82, or aa 20 to aa 556 of SEQ ID NO: 82. In further embodiments, CD19 isoform 2 comprises, or consists essentially of, or yet further consists of

(SEQ ID NO: 83)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLAGSQS
YEDMRGILYAAPQLRSIRGQPGPNHEEDADSYENMDNPDGPDPAWGGGGR
MGTWSTR.

As used herein, the term “truncation” or a grammatical variation thereof refers to a shortening in the amino acid sequence of a polypeptide or the nucleotide sequence of a nucleic acid. A protein truncation may be the result of a truncation in the nucleic acid sequence encoding the protein, a substitution or other mutation that creates a premature stop codon without shortening the nucleic acid sequence, or from alternate splicing of RNA in which a substitution or other mutation that does not itself cause a truncation results in aberrant RNA processing. In some embodiments, a truncated polypeptide of a reference polypeptide retains substantially the biological function of the reference polypeptide. In other embodiments, a truncated polypeptide of a reference polypeptide has a reduced or substantially eliminated biological function compared to that of the reference. Additionally or alternatively, a truncated polypeptide of a reference polypeptide retains one or more epitopes of the reference.

In some embodiments, the term “truncated CD19” refers to a non-functional fragment of CD19. A non-limiting example is that a portion of or the whole cytoplasmic domain of the CD19 can be truncated. Accordingly, the truncated CD19 has a reduced or substantively abolished ability of activating downstream signaling pathways, such as mediating mobilization of cytoplasmic calcium. While retaining the extracellular domain of the CD19, such truncated CD19 can still be detected by an antibody or an antigen binding fragment thereof specifically recognizing and binding to the extracellular domain of the CD19. In further embodiments, while retaining the transmembrane domain of the CD19, the truncated CD19 can still be expressed on a cell membrane, such as on the cell surface. In some embodiments, a truncated CD19 comprises, or consists essentially of, or yet further consists of any one or more of truncated CD19R (also called CD19t) having a sequence that is at least 90%, at least 95%, at least 98% identical to or identical to: MPPPRLLFFLLFLTPMEVRPEEPLVVKVEEGDNAVLQCLKGTSDGPTQQLTWSRESPLKPF LKLSLGLPGLGIHMRPLAIWLFIFNVSQQMGGFYLCQPGPPSEKAWQPGWTVNVEGSGELF RWNVSDLGGLGCGLKNRSSEGPSSPSGKLMSPKLYVWAKDRPEIWEGEPPCVPPRDSLNQ SLSQDL TMAPGSTLWLSCGVPPDSVSRGPLSWTHVHPKGPKSLLSLELKDDRPARDMWV METGLLLPRATAQDAGKYYCHRGNLTMSFHLEITARPVLWHWLLRTGGWKVSAVTLAY LIFCLCSLVGILHLQRALVLRRKR (SEQ ID NO: 26). In some cases, the truncated CD19t has 1, 2, 3, 4 of 5 amino acid changes (preferably conservative) compared to SEQ ID NO: 26.

As used herein, the term “autologous,” in reference to cells refers to cells that are isolated and infused back into the same subject (recipient or host). “Allogeneic” refers to non-autologous cells.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a spacer, e.g. a “hinge domain” which serves as a linker between the extracellular and transmembrane domains. Non limiting examples of such domains are provided herein, e.g.:

Hinge domain: IgG1 heavy chain hinge coding
sequence:
(SEQ ID NO: 84)
CTCGAGCCCAAATCTTGTGACAAAACTCACACATGCCCACCGTGCCCG.

Additional non-limiting example includes an IgG4 hinge region, IgD and CD8 domains, as known in the art.

Transmembrane domain: CD28 transmembrane region
coding sequence:
(SEQ ID NO: 85)
TTTTGGGTGCTGGTGGTGGTTGGTGGAGTCCTGGCTTGCTATAGCTTGCT
AGTAACAGTGGCCTTTATTATTTTCTGGGTG.
Intracellular domain: 4-1BB co-stimulatory
signaling region coding sequence:
(SEQ ID NO: 86)
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG.
Intracellular domain: CD28 co-stimulatory
signaling region coding sequence:
(SEQ ID NO: 87)
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC.
Intracellular domain: CD3 zeta signaling region
coding sequence:
(SEQ ID NO: 88)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “CD4 transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD4 transmembrane domain sequence as shown herein. The example sequences of CD4 transmembrane domain are provided in Parrish, Heather L et al. “A Transmembrane Domain GGxxG Motif in CD4 Contributes to Its Lck-Independent Function but Does Not Mediate CD4 Dimerization.” PloS one vol. 10,7 e0132333. 6 Jul. 2015, doi:10.1371/journal.pone.0132333. Non-limiting examples of such include the human CD4 transmembrane domain:

(SEQ ID NO: 16)
MALIVLGGVAGLLLFIGLGIFF.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain:
(SEQ ID NO: 89)
PAKPTTTPAPRPPTPAPTIASQPLSLRPEACRPAAGGAVHTRGLDFACDI
Y;
Mouse CD8 alpha hinge domain:
(SEQ ID NO: 90)
KVNSTTTKPVLRTPSPVHPTGTSQPQRPEDCRPRGSVKGTGLDFACDIY;
Cat CD8 alpha hinge domain:
(SEQ ID NO: 91)
PVKPTTTPAPRPPTQAPITTSQRVSLRPGTCQPSAGSTVEASGLDLSCDI
Y.

As used herein, the term “CD28 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS (SEQ ID NO: 92) or SEQ ID NO: 15, and equivalents of each thereof.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain(GenBank Accession No: NP_113726.1) provide additional example sequences of the CD8 α transmembrane domain. The sequences associated with each of the listed accession numbers are provided as follows:

Human CD8 alpha transmembrane domain:
(SEQ ID NO: 17), or SEQ ID NOS: 18 or 19
IYIWAPLAGTCGVLLLSLVIT.
Mouse CD8 alpha transmembrane domain:
(SEQ ID NO: 93)
IWAPLAGICVALLLSLIITLI.
Rat CD8 alpha transmembrane domain:
(SEQ ID NO: 94)
IWAPLAGICAVLLLSLVITLI.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. In some embodiments, the CAR comprises, or alternatively consists essentially of, or yet consists of a CD28 transmembrane and cytoplasmic domain comprising

FWVLVVVGGVLACYSLLVTVAFIIFWVRSKRSRLLHSDYMNMTPRRPGPTRKHYQPYAPP RDFAAYRS (SEQ ID NO: 95) or an equivalent thereof. In further embodiments, the equivalent of SEQ ID NO: 95 may comprises 1, or 2, or 3, or 4, or 5, or 6, or 7, or 8, or 9, or 10, or 11, or 12, or 13, or 14, or 15, or more mutations compared to SEQ ID NO: 95 but is still capable of functioning as a transmembrane domain and a costimulatory signaling region.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below:

4-1BB costimulatory signaling region:
(SEQ ID NO: 24), or SEQ ID NO: 20
KRGRKKLLYIFKQPFMRPVQTTQEEDGCSCRFPEEEEGGCEL.

As used herein, the term “2B4 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 2B4 costimulatory signaling region sequence shown herein.

2B4 costimulatory signaling region:
(SEQ ID NO: 66)
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYS.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide sequence provided below.

ICOS costimulatory signaling region coding
sequence:
(SEQ ID NO: 96)
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence OX40 costimulatory signaling region coding sequence: AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC (SEQ ID NO: 97), and equivalents thereof.

As used herein, the term “DAP10 costimulatory signaling region” or “DAP10 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP10 costimulatory signaling region sequence as shown herein. Non-limiting example sequences of the DAP10 costimulatory signaling region are disclosed in U.S. Pat. No. 9,587,020B2, and include the exemplary sequence: RPRRSPAQDGKVYINMPGRG (SEQ ID NO: 98), or equivalents thereof.

As used herein, the term “DAP12 costimulatory signaling region” or “DAP12 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP12 costimulatory signaling region sequence as disclosed in U.S. Pat. No. 9,587,020B2. Non-limiting example sequences of the DAP12 costimulatory signaling region are disclosed in U, and include the exemplary sequence: ESPYQELQGQRSDVYSDLNTQ (SEQ ID NO: 99), or equivalents thereof.

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. Non-limiting example sequences of the CD3 zeta signaling domain are provided in U.S. application Ser. Nos. 13/826,258, 15/327,794 and

see U.S. Pat. No. 10,801,012, e.g.:
(SEQ ID NO: 21)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR;
or
see e.g., U.S. Pat. No. 10,801,012B2
(SEQ ID NO: 100)
MRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR;
or
see e.g., U.S. Pat. Publ. No. 2017/0209492A1
(SEQ ID NO: 101)
RVKFSRSADAPAYKQGQNQLYNELNLGRREEYDVLDKRRGRDPEMGGKPR
RKNPQEGNELQKDKMAEAYSEIGMKGERRRGKGHDGQGLSTATKDTYDAL
HMQALPPR;
or
see e.g., U.S. Pat. Publ. No.: 2017/0209492A1
(SEQ ID NO: 102)
RVKFSRSADAPAYQQGQNQLYNELNLGRREEYDVLDKRRGRDPEMGGKPR
RKNPQEGNELQKDKMAEAYSEIGMKGERRRGKGHDGQGLSTATKDTYDAL
HMQALPPR.

Alternative signaling domains for CD3 zeta include signaling motifs which are known as immunoreceptor tyrosine-based activation motifs or ITAMs. Examples of ITAM containing primary cytoplasmic signaling sequences include those derived from CD3 zeta, FcR gamma, FcR beta, CD3 gamma, CD3 delta, CD3 epsilon, CD5, CD22, CD79a, CD79b, CD278 (also known as “ICOS”), FcϵRI, and CD66d. Further examples of molecules containing a primary intracellular signaling domain that are of particular use in the invention include those of DAP10, DAP12, and CD32. Non-limiting example sequences of the DAP10 costimulatory signaling region are disclosed in U.S. Patent Application US2017/0209492.

As used herein, the term “suicide gene” refers to any gene that expresses a product (optionally with presence of another agent, such as an antibody) that is fatal to the cell expressing the suicide gene. Transcription or expression of such gene, i.e., presence of its gene product, in a cell alone or together with other agents causing the cell to kill itself, for example through apoptosis. It provides a possible strategy of eliminating a cell, for example, a therapeutic cell expressing CAR after it performs its desired function, such as treating a cancer. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR (tEGFR), or inducible caspase (“iCasp”). In yet further embodiments, exemplified suicide strategy includes the thymidine kinase/ganciclovir system, the cytosine deaminase/5-fluorocytosine system, the nitroreductase/CB1954 system, carboxypeptidase G2/Nitrogen mustard system, cytochrome P450/oxazaphosphorine system, purine nucleoside phosphorylase/6-methylpurine deoxyriboside (PNP/MEP), the horseradish peroxidase/indole-3-acetic acid system (HRP/IAA), and the carboxylesterase/irinotecan (CE/irinotecan) system, the truncated EGFR (tEGFR), inducible caspase (“iCasp”), the E. coli gpt gene, the E. coli Deo gene and nitroreductase. See, more details at Karjoo, Z. et al. 2016. Adv. Drug Deliv. Rev. 99 (Pt. A):123-128.

A protein expressed on cell surface may be used as a marker (such as for purification or detection or tracking) or to provide a suicide switch of a CAR expressing cell as disclosed herein. Such protein is referred to herein as a suicide gene product or a detectable marker or both. A portion of or the whole cytoplasmic region of such protein is usually truncated so that the native function of the protein is reduced or even abolished. Thus, such a protein is also referred to herein as a truncated protein marker. In some embodiments, when used as a suicide switch of the CAR expressing cell, the truncated protein marker does not express or is expressed at a substantially lower level on a normal cell or a normal cell adjacent to the CAR expressing cell in the subject. Accordingly, upon removal of the CAR expressing cell (for example, by administering an antibody specially recognizing and binding the truncated protein marker, or by administering a toxin conjugated to a moiety directing the toxin to the truncated protein marker), a normal cell of the subject would not be jeopardized. Accordingly, in some embodiments, a method as disclosed herein can further comprise administering the subject an agent reducing or abolishing the CAR expressing cell in the subject. In further embodiments, the agent reducing or abolishing the CAR expressing cell in the subject comprises, or consists essentially of, or yet further consists of an antibody or a fragment thereof specifically recognizing and binding to the suicide gene product, such as tEGFR or RQR8. Additionally or alternatively, the administration of the agent reducing or abolishing the CAR expressing cell in the subject is about 1 day, about 3 days, about 1 week, about 2 weeks, about 3 weeks, about 4 weeks, about 5 weeks, about 6 weeks, about 7 weeks, about 8 weeks, about 9 weeks, about 10 weeks, about 3 months, about 4 months, about 5 months, about 6 months, about 1 year, about 1.5 years, about 2 years, or longer post the administration of a cell as disclosed herein.

As used herein, cytokines are a large group of proteins, peptides or glycoproteins that are secreted by specific cells of the immune system and signaling molecules that mediate and regulate immunity, inflammation and hematopoiesis. In some embodiments, they are about 5 kDa to about 20 kDa. Cytokines comprise chemokines, interferons, interleukins, lymphokines, and tumour necrosis factors, but generally not hormones or growth factors. In some embodiments, the cytokine(s) as used herein promote one or more of the development, differentiation, activation, or expansion of immune cells, such as T cells, NK cells, NKT cells, or any combination thereof.

As used herein, interleukin (IL) refers to cytokines that was first seen to be expressed by white blood cells (leukocytes). The function of the immune system depends in a large part on interleukins. The majority of interleukins are synthesized by helper CD4 T lymphocytes, as well as through monocytes, macrophages, and endothelial cells. They promote the development and differentiation of T and B lymphocytes, NK cells, NKT cells, and hematopoietic cells. As used herein, an interleukin can be a soluble cytokine secreted out of a cell, or a membrane bound (mb) cytokine expressed on a cell surface. A soluble form and a membrane bound form of a cytokine can be converted by one of skill in the art, such as by a method comprising, consisting essentially of, or consisting of engineering a transmembrane domain (such as a platelet-derived growth factor receptor beta (PDGFRP) transmembrane domain) or a signal peptide or both a transmembrane domain and a signal peptide to a cytokine.

As used herein, Interleukin-15 or IL-15 refers to a cytokine or gene encoding for a cytokine that regulates T cell and natural killer cell activation and proliferation. IL-15 induces the activation of JAK kinases, as well as the phosphorylation and activation of transcription activators STAT3, STAT5, and STAT6. IL-15 stimulates phagocytosis probably by signaling through the IL15 receptor, composed of the subunits IL15RA, IL2RB and IL2RG, which results in kinase SYK activation. IL-15 is found to bind to hematopoietic receptor subunits that are common binding sites of Interleukin-2 (IL-2). Since IL-15 and IL-2 compete for common receptors, the two molecule negatively regulate each other's activity. The number of CD8+ memory cells is shown to be controlled by a balance between this cytokine and IL-2. IL-15 may increase the expression of apoptosis inhibitor BCL2L1/BCL-x(L), possibly through the transcription activation activity of STAT6, and thus prevent apoptosis.

In some embodiments, the IL-15 is a soluble IL-15. In further embodiments, the soluble IL-15 comprises, or consists essentially of, or yet further consists of an amino acid sequence selected from

### NWVNVISDLKKIEDLIQSMHIDATLYTESDVHPSCKVTAMKCFLLELQVISLESGDASIHDT VENLIILANNSLSSNGNVTESGCKECEELEEKNIKEFLQSFVHIVQMFINTS (SEQ ID NO: 103); or

GIHVFILGCFSAGLPKTEANWVNVISDLKKIEDLIQSMHIDATLYTESDVHPSCKVTAMKCF LLELQVISLESGDASIHDTVENLIILANNSLSSNGNVTESGCKECEELEEKNIKEFLQSFVHIV QMFINTS (SEQ ID NO: 43), or an amino acid sequence of
MRISKPHLRSISIQCYLCLLLNSHFLTEAGIHVFILGCFSAGLPKTEANWVNVISDLKKIEDLI QSMHIDATLYTESDVHPSCKVTAMKCFLLELQVISLESGDASIHDTVENLIILANNSLSSNG NVTESGCKECEELEEKNIKEFLQSFVHIVQMFINTS (SEQ ID NO: 104) (each of which may be referred to herein as a wild type IL15) or an equivalent thereof. In some embodiments, the IL15 equivalent has at least about 10%, or about 20%, or about 30%, or about 40%, or about 50%, or about 60%, or about 70%, or about 80%, or about 90%, or about 95%, or about 100%, or about 1.5 fold, or about 2 fold, or about 3 fold, or about 5 fold, or about 10 fold, or more binding affinity to IL15RA compared to the wild type IL15. In some embodiments, the IL-15 equivalent is a IL-15 comprising one or more mutations selected from L45D, L45E, S51D, L52D, N72D, N72E, N72A, N72S, N72Y and N72P, wherein the first letter indicates the original amino acid residue, the last letter indicates the mutated amino acid residue and the number in the center indicates the amino acid residue number in or aligned to the sequence of

(SEQ ID NO: 103) or SEQ ID NO: 43
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS.

In some embodiments, used herein as a cytokine is a soluble complex (SIL15C) comprising, or consisting essentially of, or consisting of an IL-15 and an IL15 receptor. In further embodiments, the IL-15 comprises, or consists essentially of, or yet further consists of an amino acid sequence of GIHVFILGCFSAGLPKTEANWVNVISDLKKIEDLIQSMHIDATLYTESDVHPSCKVTAMKCF LLELQVISLESGDASIHDTVENLIILANNSLSSNGNVTESGCKECEELEEKNIKEFLQSFVHIV QMFINTS (SEQ ID NO: 43), or an amino acid sequence of MRISKPHLRSISIQCYLCLLLNSHFLTEAGIHVFILGCFSAGLPKTEANWVNVISDLKKIEDLI QSMHIDATLYTESDVHPSCKVTAMKCFLLELQVISLESGDASIHDTVENLIILANNSLSSNG NVTESGCKECEELEEKNIKEFLQSFVHIVQMFINTS (SEQ ID NO: 104) or an equivalent of each thereof.

In further embodiments, the IL15 receptor comprises, or consists essentially of, or yet further consists of an IL15 receptor subunit alpha (IL15RA) or an equivalent thereof. Additionally or alternatively, the IL15 receptor comprises, or consists essentially of, or yet further consists of an IL15 receptor subunit beta (IL15RB, i.e., IL2 receptor subunit beta). In some embodiments, the IL15RA equivalent has at least about 10%, or about 20%, or about 30%, or about 40%, or about 50%, or about 60%, or about 70%, or about 80%, or about 90%, or about 95%, or about 100%, or about 1.5 fold, or about 2 folds, or about 3 folds, or about 5 folds, or about 10 folds, or more binding affinity to IL15 compared to the wildtype IL15RA. The binding affinity can be determined by a method as provided in the Examples as well as in the art, such as Wei et al, J. Immunol, vol. 167(1), p:277-282, 2001. In some embodiments, the IL15RA equivalent comprises, or consists essentially of, or yet further consists of a sushi domain of the IL15RA. In further embodiments, the IL15RA equivalent is a fragment of the wildtype IL15RA comprising, or consisting essentially of, or yet further consisting of a sushi domain of the IL15RA. In some embodiments, the IL15 receptor comprises, or consists essentially of, or consists of

ITCPPPMSVEHADIWVKSYSLYSRERYICNSGFKRKAGTSSLTECVLNKATNVAHWTTPSL KCIR (SEQ ID NO: 80) or an equivalent thereof.

In further embodiments, the SIL15C comprises, or consists essentially of, or yet further consists of an amino acid sequence of

GIHVFILGCFSAGLPKTEANWVNVISDLKKIEDLIQSMHIDATLYTESDVHPSCKVTAMKCF LLELQVISLESGDASIHDTVENLIILANNSLSSNGNVTESGCKECEELEEKNIKEFLQSFVHIV QMFINTS (soluble IL-15, SEQ ID NO: 43) or an equivalent thereof and an amino acid sequence of ITCPPPMSVEHADIWVKSYSLYSRERYICNSGFKRKAGTSSLTECVLNKATNVAHWTTPSL KCIR (SEQ ID NO: 80) or an equivalent thereof. In some embodiments, the IL15 and the IL15 receptor are linked by a peptide linker. In some embodiments, the linker comprises SGGGSGGGGSGGGGSGGGGSGGGSLQ (SEQ ID NO: 71) or GGGGSGGGGSGGGGS (SEQ ID NO: 37), or an equivalent of each thereof. In some embodiments, the SIL15C comprises, or consists essentially of, or yet further consists of ALT-803 (IL15N72D:IL15RαSu/IgG1 Fc complex). ALT-803 is an IL-15 super agonist complex IL-15N72D:IL-15RαSu/Fc that comprises, or consists essentially of, or yet further consists of an IL-15 mutant (IL-15N72D) and a dimeric IL-15 receptor α sushi domain-IgG1 Fc fusion protein.

As used herein, the IL15RA sushi domain refers to a consensus sequence of CPXiPX2SVEHADIX3VKSYSLX4SRERYX5CNSGFKRKAGTSSLTECVLNKATNX6AX7WTTP SLKC, wherein X₁ is P or A, .X₂ is M or V, X₃ is W, R or Q, X₄ is Y or H, X₅ is I or V, X₆ is V or A, and X₇ is V or A (SEQ ID NO: 105).

As used herein, the terms “IL15RA”, “IL 15 RA”, “IL-15 RA” and “IL 15 Receptor Subunit Alpha” are used interchangeably to refer to a cytokine receptor that specifically binds interleukin 15 (IL15) with high affinity and encoded by the IL15RA gene or a fragment thereof. IL15RA can signal both in cis and trans where IL15 receptor (IL15R) from one subset of cells presents IL15 to neighboring IL2RG-expressing cells. Non-limiting exemplary sequences of this protein or the underlying gene or functions thereof may be found under Gene Cards ID: GC10M005943, HGNC: 5978, Entrez Gene: 3601, Ensembl: ENSG00000134470, OMIM: 601070, or UniProtKB: Q13261, each of which is incorporated by reference herein in its entirety. In some embodiments, the IL15RA comprises, or consists essentially of, or yet further consists of an amino acid sequence of

(SEQ ID NO: 80)
ITCPPPMSVEHADIWVKSYSLYSRERYICNSGFKRKAGTSSLTECVLNKA
TNVAHWTTPSLKCIR.

As used herein, IL2RB and IL15RB are used interchangeably to refer to a receptor for IL2 and in association with IL15RA, involved in the stimulation of neutrophil phagocytosis by IL15. See, for example, Ratthe et al. J. Leukoc. Biol. 76:162-168(2004). Non-limiting exemplary sequences of this protein or the underlying gene or functions thereof may be found under Gene Cards ID: GC22M037125, HGNC: 6009, Entrez Gene: 3560, Ensembl: ENSG00000100385, OMIM: 146710, or UniProtKB: P14784, each of which is incorporated by reference herein in its entirety.

In other embodiments, the IL-15 is a membrane bound IL-15 (mbIL-15), such as a soluble IL-15 conjugated with a transmembrane domain optionally further comprising a peptide linker or one or more random amino acid residues, and thus bounded on cell membrane. In further embodiments, the mbIL-15 is IL15 conjugated with a PDGFRP transmembrane domain or an equivalent thereof.

A signal peptide, as used herein, refers to (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined toward the secretory pathway. In one embodiment, the signal peptide is a secretary signal.

In some embodiments, the signal peptide comprises, or consists essentially of, or yet further consists of MYRMQLLSCIALSLALVTNS (SEQ ID NO: 31), or MWLQSLLLLGTVACSIS (SEQ ID NO: 106), or MRISKPHLRSISIQCYLCLLLNSHFLTEA (SEQ ID NO: 107), or MRSSPGNMERIVICLMVIFLGTLV (SEQ ID NO: 108), or MGWSSIILFLVATATGVH (SEQ ID NO: 30), or MLLLVTSLLLCELPHPAFLLIP (SEQ ID NO: 36) or an equivalent of each thereof. In one embodiment, the signal peptide comprises, or consists essentially of, or yet further consists of MYRMQLLSCIALSLALVTNS (SEQ ID NO: 31), or MWLQSLLLLGTVACSIS (SEQ ID NO: 106), or MRISKPHLRSISIQCYLCLLLNSHFLTEA (SEQ ID NO: 107), or MRSSPGNMERIVICLMVIFLGTLV (SEQ ID NO: 108), or an equivalent of each thereof. In another embodiments, the signal peptide comprises, or consists essentially of, or yet further consists of MGWSSIILFLVATATGVH (SEQ ID NO: 30), or MLLLVTSLLLCELPHPAFLLIP (SEQ ID NO: 36) or an equivalent of each thereof. In one embodiment, the signal peptide is a secretary signal.

A secretary signal intends a secretory signal peptide that allows the export of a protein from the cytosol into the secretory pathway. Proteins can exhibit differential levels of successful secretion and often certain signal peptides can cause lower or higher levels when partnered with specific proteins. In eukaryotes, the signal peptide is a hydrophobic string of amino acids that is recognized by the signal recognition particle (SRP) in the cytosol of eukaryotic cells. After the signal peptide is produced from an mRNA-ribosome complex, the SRP binds the peptide and stops protein translation. The SRP then shuttles the mRNA/ribosome complex to the rough endoplasmic reticulum where the protein is translated into the lumen of the endoplasmic reticulum. The signal peptide is then cleaved off the protein to produce either a soluble, or membrane tagged (if a transmembrane region is also present), protein in the endoplasmic reticulum. These are known in the art, and commercially available from vendors, e.g., Oxford Genetics.

As used herein, a “ribosomal skip sequence” refers to a sequence that prevents a ribosome from covalently linking a new inserted amino acid molecule during translation. Ribosomal skipping can be caused by the introduction of a cleavable peptide to form multiple peptides by causing a ribosome to fail at making a peptide. A non-limiting example includes a polypeptide that is at least 95% identical or identical to LEGGGEGRGSLLTCGDVEENPGPR; SEQ ID NO: 27). Other ribosomal skip sequences useful in a CAR or peptide described herein include T2At having a sequence that is at least 95% identical or identical to: EGRGSLLTCGDVEENPGP (SEQ ID NO: 46) or P2A having a sequence that is at least 95% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NOS: 46 or 47.

As used herein, a “cleavable peptide”, which is also referred to as a cleavable linker, means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X—N-P-G-P, wherein X refers to any amino acid generally thought to be self-cleaving (SEQ ID NO: 109). Additional examples are provided herein and incorporated by reference.

As used herein the terms “linker sequence” “linker peptide” and “flexible linker” are used interchangeably, relating to any amino acid sequence comprising from 1 to 10, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (Glycine₄Serine)₃ (SEQ ID NO: 37) flexible polypeptide linker comprising three copies of gly-gly-gly-gly-ser (SEQ ID NO: 39). In some embodiments, the linker sequence is a (G4S)ₙ (SEQ ID NO: 110), wherein n is 1, or 2, or 3, or 4, or 5, or 6, or 7, or 8, or 9, or 10, or 11, or 12, or 13, or 14, or 15. In some embodiments, the linker comprises SGGGSGGGGSGGGGSGGGGSGGGSLQ (SEQ ID NO: 71) or GGGGSGGGGSGGGGS (SEQ ID NO: 37), or an equivalent of each thereof.

The term “internal ribosome entry site” or “IRES” as used herein interchangeably refers to a polynucleotide that directly promotes ribosome binding and mRNA translation and thereby permits initiation of translation in cap-independent manner. In some embodiments, an IRES refers an RNA sequence on a messenger RNA (mRNA). Additionally or alternatively, an IRES also refers to a polynucleotide sequence (such as an RNA sequence, a DNA sequence or a hybrid thereof) complementary, or reverse, or both complementary and reverse to an IRES RNA sequence. Non-limiting examples of IRES can be found in Hellen CU and Sarnow P. Internal ribosome entry sites in eukaryotic mRNA molecules. Genes Dev. 2001 Jul. 1; 15(13):1593-612.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-histidine (His) tag, calmodulin binding protein (CBP), or maltose-binding protein (MBP)), or a fluorescent tag. In one aspect, truncated CD19 and EGFR are useful as markers.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein.

Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “vector” refers to a nucleic acid construct deigned for transfer between different hosts, including but not limited to a plasmid, a virus, a cosmid, a phage, a BAC, a YAC, etc. Non-limiting examples of plasmids include RD114, FD114-TR and VSVG. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV 11, AAV12, AAV13, AAV PHP.B, or AAV rh74. These vectors are commercially available or have been described in the patent or technical literature.

The term “transduce” or “transduction” as it is applied to the production of chimeric antigen receptor cells refers to the process whereby a foreign nucleotide sequence is introduced into a cell.

In some embodiments, this transduction is done via a vector.

As used herein, a cell may be a prokaryotic or a eukaryotic cell. In further embodiments, the cell is an immune cell.

As used herein, “an immune cell” includes, e.g., white blood cells (leukocytes, such as granulocytes (neutrophils, eosinophils, and basophils), monocytes, and lymphocytes (T cells, B cells, natural killer (NK) cells and NKT cells)) which may be derived from hematopoietic stem cells (HSC) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSc), hematopoietic stem cells (HSCs), or immortalized cells. In some embodiments, the HSC are derived from umbilical cord blood of a subject, peripheral blood of a subject, or bone marrow of a subject.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Non-limiting examples include 293, 293T cells and immune cells.

An “enriched population” of cells intends a substantially homogenous population of cells having certain defined characteristics. The cells are greater than 70%, or alternatively greater than 75%, or alternatively greater than 80%, or alternatively greater than 85%, or alternatively greater than 90%, or alternatively greater than 95%, or alternatively greater than 98% identical in the defined characteristics.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and-4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK—YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™) DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5,-6,-7,-8,-9,-10, and-16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-l. Non-limiting exemplary sources for such commercially available cell lines include the American Type Culture Collection, or ATCC, (http://www.atcc.org/) and the German Collection of Microorganisms and Cell Cultures (https://www.dsmz.de/).

The term “stem cell” refers to a cell that is in an undifferentiated or partially differentiated state and has the capacity for self-renewal or to generate differentiated progeny or both. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural or neuronal stem cells (NSCs). In some embodiments, the stem or progenitor cells can be embryonic stem cells or an induced pluripotent stem cell (iPSC). In some embodiments, the stem or progenitor cells are hematopoietic stem cells (HSCs). As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, immune or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, a “pluripotent cell” defines a less differentiated cell that can give rise to at least two distinct (genotypically or phenotypically or both) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, the term “pluripotent gene or marker” intends an expressed gene or protein that has been correlated with an immature or undifferentiated phenotype, e.g., Oct 3/4, Sox2, Nanog, c-Myc and LIN-28. Methods to identify such are known in the art and systems to identify such are commercially available from, for example, EMID Millipore (MILLIPLEX® Map Kit).

As used herein, hematopoietic stem cells (HSCs) are cells, such as stem cells, that give rise to all types of blood cells, including but not limited to white blood cells, red blood cells, and platelets. Hematopoietic stem cells can be found in the peripheral blood and the bone marrow. In some embodiments, an immune cell as disclosed herein is derived from an HSC.

The term “phenotype” refers to a description of an individual's trait or characteristic that is measurable and that is expressed only in a subset of individuals within a population. In one aspect of the disclosure, an individual's phenotype includes the phenotype of a single cell, a substantially homogeneous population of cells, a population of differentiated cells, or a tissue comprised of a population of cells.

In some embodiments, a population of cells intends a collection of more than one cell that is identical (clonal) or non-identical in phenotype or genotype or both. The population can be purified, highly purified, substantially homogenous or heterogeneous as described herein.

The terms effective period (or time) and effective conditions refer to a period of time or other controllable conditions (e.g., temperature, humidity for in vitro or ex vivo methods), necessary or preferred for an agent or composition to achieve its intended result, e.g., the differentiation or dedifferentiation of cells to a pre-determined cell type.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively more than 95%, of the cells are of the same or similar phenotype. Phenotype can be determined by a pre-selected cell surface marker or other marker.

As used herein, “treating” or “treatment” of a disease in a subject and the like are used herein to mean obtaining a desired pharmacologic and/or physiologic effect. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, such as a cancer. In some embodiments, it refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, the term “treatment” or “treating” excludes prevention or prophylaxis.

As used herein, the term “sample” and “biological sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. In some embodiments, a biological sample is selected from peripheral blood, plasma or serum.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, a therapeutic protein or polypeptide refers to a protein or a polypeptide suitable for a treatment, including but not limited to an antibody or a fragment thereof, an enzyme, a ligand or a receptor. Such therapeutic protein or polypeptide may be chose by a physician or one of skill in the art, based on the disease to be treated. For example, for treating a cancer, an antibody to an immune checkpoint receptor or a ligand thereof may be used, such as an anti-PD-1 antibody or an anti-PD-L1 antibody or both.

In one embodiment, the term “disease” or “disorder” as used herein refers to a cancer or tumor (which are used interchangeably), a status of being diagnosed with such disease, a status of being suspect of having such disease, or a status of at high risk of having such disease.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen. In some embodiments, the term “cancer” as used herein refers to multiple myeloma (MM). In some embodiments, the term “cancer” as used herein refers to acute myeloid leukemia (AML). Additionally or alternatively, the cancer as used herein expresses PSCA. In some embodiment, the cancer is a relapsed cancer. In some embodiments, the cancer is a refractory cancer.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). The term pharmaceutically acceptable carrier (or medium), which may be used interchangeably with the term biologically compatible carrier or medium, refers to reagents, cells, compounds, materials, compositions, or dosage forms, or any combination thereof, that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit to risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodible). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result or protection or both desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules or other entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an intratumoral administration, or administration to a tumor microenvironment, or both. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

“Administration” can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. In some embodiments, 1×10⁴ to 1×10¹⁵ or ranges in between of cells as disclosed herein are administrated to a subject, such as 1×107 to 1×10¹⁰. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

An agent of the present disclosure can be administered for therapy by any suitable route of administration. It will also be appreciated that the optimal route will vary with the condition and age of the recipient, and the disease being treated.

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

The term “suffering” as it related to the term “treatment” refers to a patient or individual who has been diagnosed with or is predisposed to a disease as disclosed herein. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro, or ex vivo, or in vivo tests (or any combination thereof) initially can provide useful guidance on the proper doses for patient administration. In general, one will desire to administer an amount of the agent as disclosed herein (such as a cell) that is effective to achieve a serum level commensurate with the concentrations found to be effective in vitro or ex vivo. Determination of these parameters is well within the skill of the art. These considerations, as well as effective formulations and administration procedures are well known in the art and are described in standard textbooks.

“Therapeutically effective amount” of a drug or an agent refers to an amount of the drug or the agent (such as a cell as disclosed herein) that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses, as needed to induce a partial or complete effect. Thus, a therapeutically effective amount may be administered in one or more administrations. In some embodiments, a therapeutically effective amount of cells as disclosed herein is 1×10⁴ to 1×10¹⁵ or ranges, such as 1×10⁷ to 1×10¹⁰.

In some embodiments, a treatment, such as an immune cell comprising a polypeptide as disclosed herein, is administered to a subject as disclosed herein in an effective amount. In further embodiments, a treatment, such as an immune cell comprising a polypeptide as disclosed herein, is administered to a subject as disclosed herein in a therapeutically effective amount.

An “anti-cancer therapy,” as used herein, includes but is not limited to surgical resection, chemotherapy, cryotherapy, radiation therapy, immunotherapy and targeted therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

In some embodiments where the cancer is an immune cell cancer, an anti-cancer therapy may comprises, or consists essentially of, or consists of a hematopoietic stem cell transplantation.

In some embodiments, a therapeutic agent, such as a cell as disclosed herein, may be combined in treating a cancer with another anti-cancer therapy or a therapy depleting an immune cell. For example, lymphodepletion chemotherapy is performed followed by administration of a cell as disclosed herein, such as four weekly infusions. In further embodiments, these steps may be repeated for once, twice, three or more times until a partial or complete effect is observed or a clinical end point is achieved.

Gemcibabine (Gemzar®) is an antimetabolite used to treat carcinomas and has been used as a first-line treatment for pancreatic cancer, and in combination with cisplatin for advanced or metastatic bladder cancer and advanced or metastatic non-small cell lung cancer. It is used as a second-line treatment in combination with carboplatin for ovarian cancer and in combination with paclitaxel for breast cancer that is metastatic or cannot be surgically removed. It is commercially available from Lilly Medical.

Aldoxorubicin is a tumor-targeted doxorubicin conjugate in development by CytRx. It is the (6-maleimidocaproyl) hydrazone of doxorubicin. Essentially, this chemical name describes doxorubicin attached to an acid-sensitive linker (N-E-maleimidocaproic acid hydrazide, or EMCH).

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

### MODES FOR CARRYING OUT THE DISCLOSURE

### Nucleic Acid Molecules and Polynucleotides

This disclosure provides a first nucleic acid molecule encoding a chimeric antigen receptor (CAR) that binds to a PSCA cancer or tumor antigen, the CAR comprising, or consisting essentially of, or consisting of, a cell activation moiety comprising an extracellular, transmembrane, and intracellular domain (also referred to herein as cytoplasmic domain). The extracellular domain comprises a target-specific binding element otherwise referred to as the antigen binding domain. The intracellular domain or cytoplasmic domain comprises one or more costimulatory signaling region(s) and a signaling domain, such as a CD3 zeta chain portion. The CAR may optionally further comprise one or more of a signal peptide, flexible linker and/or a spacer domain of up to 300 amino acids, preferably 10 to 100 amino acids, more preferably 25 to 50 amino acids. The nucleic acid molecule can be DNA or RNA.

In one aspect, the present disclosure provides methods for making and using natural killer (NK) cells or other immune cells expressing a PSCA targeted chimeric antigen receptor (CAR) (also herein called PSCA CAR NK cells) co-expressing an IL-15 domain (e.g., at least a portion of IL-15, at least a portion of IL-15Ra, or a fusion protein that includes at least a portion of IL-15 and at least a portion of IL-15Ra) to treat a variety of solid tumors, (e.g., pancreatic cancer, prostate cancer, and urinary bladder cancer). The PSCA CAR NK cells described herein possess potent antigen-specific anti-tumor efficacy in vitro and in vivo. The PSCA CAR NK cells described herein also possess the potent antigen-specific anti-tumor efficacy.

In one aspect, described herein is a nucleic acid molecule comprising a first nucleic acid molecule encoding a chimeric antigen receptor (CAR) or polypeptide, wherein the chimeric antigen receptor or polypeptide comprises: an antibody single chain variable fragment (scFv) targeting PSCA, a spacer (e.g., a hinge domain), a transmembrane domain, a co-stimulatory domain, and a CD3 ξ signaling domain. The nucleic acid molecule further comprises a nucleotide molecule encoding an IL-15 domain. Non-limiting examples of an IL-15 domain can include, e.g., soluble IL-15 (sIL-15 or s15), membrane bound IL-15 (mbIL-15 or mIL-15 or m15), a fusion protein that includes soluble IL-15 and at least a portion of IL-15Rα (sIL-15c or s15c), and a fusion protein that includes a transmembrane domain, at least a portion of IL-15 and at least a portion of IL-15a (mbIL-15c or mIL-15c or m15c) that can optionally be codon-optimized.

PSCA ScFV

In one aspect, the scFv nucleic acid molecule of the first molecule encodes a heavy chain (HC) complementarity-determining region (CDR) 1 (CDRH1) comprising DYYI (aa 31 to aa 34 of SEQ ID NO: 33), an HC CDR 2 (CDRH2) comprising WIDPENGDTEFVPKFQG (aa 50 to aa 66 of SEQ ID NO: 33), and an HC CDR 3 (CDRH3) comprising GGF (aa 99 to aa 101 of SEQ ID NO: 33).

In another aspect, the scFv nucleic acid molecule of the first molecule encodes a light chain (LC) complementarity-determining region (CDR) 1 (CDRL1) comprising SASSSVRFIH (aa 24 to aa 33 of SEQ ID NO: 32), an LC CDR 2 (CDRL2) comprising DTSKLAS (aa 49 to aa 55 of SEQ ID NO: 32), and an LC CDR 3 (CDRL3) comprising QQWGSSPFT (aa 88 to aa 96 of SEQ ID NO: 32).

In a further aspect, the scFv nucleic acid molecule of the first molecule comprises a CDRH1 comprising DYYI (aa 31 to aa 34 of SEQ ID NO: 33), a CDRH2 comprising WIDPENGDTEFVPKFQG (aa 50 to aa 66 of SEQ ID NO: 33), a CDRH3 comprising GGF (aa 99 to aa 101 of SEQ ID NO: 33), a CDRL1 comprising SASSSVRFIH (aa 24 to aa 33 of SEQ ID NO: 32), a CDRL2 comprising DTSKLAS (aa 49 to aa 55 of SEQ ID NO: 32), and a CDRL3 comprising QQWGSSPFT (aa 88 to aa 96 of SEQ ID NO: 32).

In a yet further aspect, the scFv nucleic acid molecule of the first molecule encodes a light chain variable region of SEQ ID NO: 34 or an equivalent thereof, and a heavy chain variable region of SEQ ID NO: 35 or an equivalent thereof.

In one embodiment, the scFv nucleic acid molecule of the first molecule encodes the amino acid sequence of SEQ ID NO: 35, or an equivalent of each thereof, or a CDRH1 comprising SYSMS (aa 31 to aa 35 of SEQ ID NO: 35), a CDRH2 comprising YINDSGGSTFYPDTVKG (aa 50 to aa 66 of SEQ ID NO: 35), and a CDRH3 comprising RMYYGNSHWHFDV (aa 99 to aa 111 of SEQ ID NO: 35).

In a further embodiment, the scFv nucleic acid molecule of the first molecule encodes a CDRL1 comprising GTSQDINNYLN (aa 24 to aa 34 of SEQ ID NO: 34), a CDRL2 comprising YTSRLHS (aa 50 to aa 56 of SEQ ID NO: 34), and a CDRL3 comprising QQSKTLPWT (aa 89 to aa 97 of SEQ ID NO: 34).

In a yet further embodiment, the scFv nucleic acid molecule of the first molecule encodes a CDRH1 comprising SYSMS (aa 31 to aa 35 of SEQ ID NO: 35), a CDRH2 comprising YINDSGGSTFYPDTVKG (aa 50 to aa 66 of SEQ ID NO: 35), a CDRH3 comprising RMYYGNSHWHFDV (aa 99 to aa 111 of SEQ ID NO: 35), a CDRL1 comprising GTSQDINNYLN (aa 24 to aa 34 of SEQ ID NO: 34), a CDRL2 comprising YTSRLHS (aa 50 to aa 56 of SEQ ID NO: 34), and a CDRL3 comprising QQSKTLPWT (aa 89 to aa 97 of SEQ ID NO: 34).

In another aspect, the scFv nucleic acid molecule of the first molecule encodes a light chain variable region of SEQ ID NO: 34 or an equivalent thereof, and a heavy chain variable region of SEQ ID NO: 35 or an equivalent thereof.

In another aspect, the scFv nucleic acid molecule further comprise a polynucleotide encoding a flexible linker polypeptide between the HC and the LC. In some embodiments, a useful flexible linker is 1, 2, 3, 4, 5, 6, 7, 8, 9, or 10 repeats of the sequence GGGS (SEQ ID NO: 38, repeats disclosed as SEQ ID NO: 111). In some embodiments, a useful flexible linker is 1, 2, 3, 4, 5, 6, 7, 8, 9, or 10 repeats of the sequence GGGGS (SEQ ID NO: 39, repeats disclosed as SEQ ID NO: 112). A useful linker could comprise (G4S)₃ GGGGSGGGGSGGGGS (SEQ ID NO: 37). In one aspect, the scFv comprises HC—linker—LC, or alternatively LC—linker—HC.

In another aspect, the scFv nucleic acid molecule of the first molecule encodes the amino acid sequence of SEQ ID NO: 1, or an equivalent of each thereof.

In another aspect, the scFv nucleic acid molecule of the first molecule encodes the amino acid sequence of SEQ ID NO: 40, or an equivalent of each thereof.

Spacer Region

The first nucleic acid encoding the CAR or polypeptide described herein can include a nucleic acid molecule encoding a spacer located between the PSCA targeting domain (i.e., a PSCA targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG |
| | | (SEQ ID NO: 2) |
| IgG4 | 12 aa | ESKYGPPCPPCP |
| hinge | | (SEQ ID NO: 3) |
| (S→P) | | |
| (S228P) | | |
| IgG4 | 12 aa | ESKYGPPCPSCP |
| hinge | | (SEQ ID NO: 4) |
| IgG4 | 22 aa | ESKYGPPCPPCPGGGSSGGGSG |
| hinge | | (SEQ ID NO: 5) |
| (S228P) + | | |
| linker | | |
| CD28 | 39 aa | IEVMYPPPYLDNEKSNGTIIHVKGK |
| hinge | | HLCPSPLFPGPSKP |
| | | (SEQ ID NO: 6) |
| CD8 | 48 aa | AKPTTTPAPRPPTPAPTIASQPLSL |
| hinge- | | RPEACRPAAGGAVHTRGLDFACD |
| 48 aa | | (SEQ ID NO: 7) |
| CD8 | 45 aa | TTTPAPRPPTPAPTIASQPLSLRPE |
| hinge- | | ACRPAAGGAVHTRGLDFACD |
| 45 aa | | (SEQ ID NO: 8) |
| IgG4 | 129 aa | ESKYGPPCPPCPGGGSSGGGSGGQP |
| (HL-CH3) | | REPQVYTLPPSQEEMTKNQVSLTCL |
| Also | | VKGFYPSDIAVEWESNGQPENNYKT |
| called | | TPPVLDSDGSFFLYSRLTVDKSRWQ |
| IgG4 | | EGNVFSCSVMHEALHNHYTQKSLSL |
| (HL-ΔCH2) | | SLGK |
| (includes | | (SEQ ID NO: 9) |
| S228P in | | |
| hinge) | | |
| IgG4 | 229 aa | ESKYGPPCPSCPAPEFEGGPSVFLF |
| (L235E, | | PPKPKDTLMISRTPEVTCVVVDVSQ |
| N297Q) | | EDPEVQFNWYVDGVEVHNAKTKPRE |
| | | EQFQSTYRVVSVLTVLHQDWLNGKE |
| | | YKCKVSNKGLPSSIEKTISKAKGQP |
| | | REPQVYTLPPSQEEMTKNQVSLTCL |
| | | VKGFYPSDIAVEWESNGQPENNYKT |
| | | TPPVLDSDGSFFLYSRLTVDKSRWQ |
| | | EGNVFSCSVMHEALHNHYTQKSLSL |
| | | SLGK |
| | | (SEQ ID NO: 10) |
| IgG4 | 229 aa | ESKYGPPCPPCPAPEFEGGPSVFLF |
| (S228P, | | PPKPKDTLMISRTPEVTCVVVDVSQ |
| L235E, | | EDPEVQFNWYVDGVEVHNAKTKPRE |
| N297Q) | | EQFQSTYRVVSVLTVLHQDWLNGKE |
| | | YKCKVSNKGLPSSIEKTISKAKGQP |
| | | REPQVYTLPPSQEEMTKNQVSLTCL |
| | | VKGFYPSDIAVEWESNGQPENNYKT |
| | | TPPVLDSDGSFFLYSRLTVDKSRWQ |
| | | EGNVFSCSVMHEALHNHYTQKSLSL |
| | | SLGK |
| | | (SEQ ID NO: 11) |
| IgG4(CH3) | 107 aa | GQPREPQVYTLPPSQEEMTKNQVSL |
| Also | | TCLVKGFYPSDIAVEWESNGQPENN |
| called | | YKTTPPVLDSDGSFFLYSRLTVDKS |
| IgG4 | | RWQEGNVFSCSVMHEALHNHYTQKS |
| (ΔCH2) | | LSLSLGK |
| | | (SEQ ID NO: 12) |
| IgG1 | 16 aa | LEPKSCDKTHTCPPCP |
| Hinge | | (SEQ ID NO: 44) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fe hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The spacer region can also comprise an IgG4 hinge region having the sequence ESKYGPPCPSCP (SEQ ID NO: 4) or ESKYGPPCPPCP (SEQ ID NO: 3). The spacer region can also comprise the hinge sequence ESKYGPPCPPCP (SEQ ID NO: 3) followed by the linker sequence GGGSSGGGSG (SEQ ID NO: 2) followed by IgG4 CH3 sequence

(SEQ ID NO: 12)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

Thus, the entire spacer region can comprise the sequence:

(SEQ ID NO: 9)
ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

Transmembrane Domain

A variety of nucleic acid molecules encoding the transmembrane domain of the CARs can be used in the CAR. In some aspects, a spacer is located carboxy terminal to the spacer region.

In some cases, the transmembrane domain nucleic acid molecule encodes a domain selected from a CD4 transmembrane domain, a CD8 transmembrane domain, a CD28 transmembrane domain, or a NKG2D transmembrane domain. In one aspect, the transmembrane domain encodes an amino acid sequence of SEQ ID NOS: 13-20 or 65, or an equivalent of each thereof. In some cases, the transmembrane domain has 1, 2, 3, 4 of 5 amino acid changes (preferably conservative) compared to SEQ ID NOS: 13-20 or 65, respectively.

In one aspect, the nucleic acid molecule encodes a CD28 transmembrane domain that includes a sequence that is at least 90%, at least 95%, at least 98% identical to or identical to: FWVLVVVGGVLACYSLLVTVAFIIFWV (SEQ ID NO: 14). In some cases, the CD28 transmembrane domain has 1, 2, 3, 4 of 5 amino acid changes (preferably conservative) compared to SEQ ID NO: 14.

Table 2 includes additional examples of suitable transmembrane domains. Where a spacer region is present, the transmembrane domain (TM) is located carboxy terminal to the spacer region.

TABLE 2
Examples of Transmembrane Domains

| | Acces- | | |
|---|---|---|---|
| Name | sion | Length | Sequence |
| CD3z | J04132.1 | 21 aa | LCYLLDGILFIYGVILTALFL |
| | | | (SEQ ID NO: 13) |
| CD28 | NM_ | 27 aa | FWVLVVVGGVLACYSLLVTVA |
| | 006139 | | FIIFWV |
| | | | (SEQ ID NO: 14) |
| CD28 | NM_ | 28 aa | MFWVLVVVGGVLACYSLLVTV |
| (M) | 006139 | | AFIIFWV |
| | | | (SEQ ID NO: 15) |
| CD4 | M35160 | 22 aa | MALIVLGGVAGLLLFIGLGIF |
| | | | F |
| | | | (SEQ ID NO: 16) |
| CD8tm | NM_ | 21 aa | IYIWAPLAGTCGVLLLSLVIT |
| | 001768 | | (SEQ ID NO: 17) |
| CD8tm2 | NM_ | 23 aa | IYIWAPLAGTCGVLLLSLVIT |
| | 001768 | | LY |
| | | | (SEQ ID NO: 18) |
| CD8tm3 | NM_ | 24 aa | IYIWAPLAGTCGVLLLSLVIT |
| | 001768 | | LYC |
| | | | (SEQ ID NO: 19) |
| 41BB | NM_ | 27 aa | IISFFLALTSTALLFLLFF |
| | 001561 | | LTLRFSVV |
| | | | (SEQ ID NO: 20) |
| NKG2D | NM_ | 21 aa | PFFFCCFIAVAMGIRFIIMVA |
| | 007360 | | (SEQ ID NO: 65) |

Costimulatory Domain

In the CARs of this disclosure, one or more polynucleotides encoding one or more costimulatory domains can be used, non-limiting examples of such include CD28, 4-1BB, 2B4, OX40, DAP10, or DAP12 or a variant thereof having 1-5 (e.g., 1 or 2) amino acid modifications. In a further aspect, the costimulatory domain comprises one or more of CD28, 4-1BB, OX40, or 2B4 costimulatory domains or a variant thereof having 1-5 (e.g., 1 or 2) amino acid modifications.

In a further aspect, the polynucleotide encodes a domain that is suitable for use with a CD3(signaling domain. In some cases, the co-signaling domain is a CD28 co-signaling or costimulatory domain that includes a sequence that is at least 90%, at least 95%, at least 98% identical to or identical to: RSKRSRLLHSDYMNMTPRRPGPTRKHYQPYAPPRDFAAYRS (SEQ ID NO: 22). In some cases, the 4-1BB co-signaling or costimulatory domain has 1, 2, 3, 4 of 5 amino acid changes (preferably conservative) compared to SEQ ID NO: 20, 23 or 24.

The costimulatory domain(s) are located between the transmembrane domain and the CD3(signaling domain. Table 3 includes additional examples of suitable costimulatory domains together with the sequence of the CD3ξ signaling domain.

TABLE 3
CD3ζ Domain and Examples of Costimulatory
Domains

| | Acces- | | |
|---|---|---|---|
| Name | sion | Length | Sequence |
| CD3ζ | J04132.1 | 113 aa | RVKFSRSADAPAYQQGQNQLYNEL |
| | | | NLGRREEYDVLDKRRGRDPEMGGK |
| | | | PRRKNPQEGLYNELQKDKMAEAYS |
| | | | EIGMKGERRRGKGHDGLYQGLSTA |
| | | | TKDTYDALHMQALPPR |
| | | | (SEQ ID NO: 21) |
| CD28 | NM_ | 42 aa | RSKRSRLLHSDYMNMTPRRPGPTR |
| | 006139 | | KHYQPYAPPRDFAAYRS |
| | | | (SEQ ID NO: 22) |
| CD28gg* | NM_ | 42 aa | RSKRSRGGHSDYMNMTPRRPGPTR |
| | 006139 | | KHYQPYAPPRDFAAYRS |
| | | | (SEQ ID NO: 23) |
| 41BB | NM_ | 42 aa | KRGRKKLLYIFKQPFMRPVQTTQE |
| | 001561 | | EDGCSCRFPEEEEGGCEL |
| | | | (SEQ ID NO: 24) |
| OX40 | NM_ | 42 aa | ALYLLRRDQRLPPDAHKPPGGGSF |
| | 003327 | | RTPIQEEQADAHSTLAKI |
| | | | (SEQ ID NO: 25) |
| 2B4 | NM_ | 120 aa | WRRKRKEKQSETSPKEFLTIYEDV |
| | 016382 | | KDLKTRRNHEQEQTFPGGGSTIYS |
| | | | MIQSQSSAPTSQEPAYTLYSLIQP |
| | | | SRKSGSRKRNHSPSFNSTIYEVIG |
| | | | KSQPKAQNPARLSRKELENFDVYS |
| | | | (SEQ ID NO: 66) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-11B1 costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-11B1 costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-11B1 co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3 signaling domain and a short linker consisting of 2-10, e.g., 3 amino acids (e.g., GGG) is can be positioned between the costimulatory domain and the CD3 ξ signaling domain. CD3ξ Signaling Domain

The first nucleic acid molecule also comprises a polynucleotide encoding a signaling domain such as a CD3ξ signaling domain or any domain that is suitable substitute or for use with a CD3ξ signaling domain. In some cases, the CD3ξ signaling domain includes a sequence that is at least 90%, at least 95%, at least 98% identical to or identical to:

RVKFSRSADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO: 21). In some cases, the CD3ξ signaling has 1, 2, 3, 4 of 5 amino acid changes (preferably conservative) compared to SEQ ID NO: 21.

Self-Cleaving Peptide

In some aspects, the first nucleic acid molecule further comprises, or consists essentially of, or consists of a polynucleotide encoding self-cleaving peptide located between the CAR and the IL-15 domain. Non-limiting examples of such include a 2A self-cleaving peptide. 2A self-cleaving peptide refers to a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

Signal Peptide

The first nucleic acid molecule encoding the CAR can further comprise a polynucleotide encoding a signal peptide, e.g., a human GM-CSF receptor alpha signal sequence
(MLLLVTSLLLCELPHPAFLLIP; SEQ ID NO: 36); a IgGk signal peptide
(METDTLLLWVLLLWVPGSTG; SEQ ID NO: 29); a IgG2 signal peptide
(MGWSSIILFLVATATGVH; SEQ ID NO: 30); a IL-2 signal peptide
(MYRMQLLSCIALSLALVTNS; SEQ ID NO: 31) or an equivalent of each thereof.

In some embodiments, the signal peptide comprises, or consists essentially of, or yet further consists of MYRMQLLSCIALSLALVTNS (SEQ ID NO: 31), or MWLQSLLLLGTVACSIS (SEQ ID NO: 106), or MRISKPHLRSISIQCYLCLLLNSHFLTEA (SEQ ID NO: 107), or MRSSPGNMERIVICLMVIFLGTLV (SEQ ID NO: 108), or MGWSSIILFLVATATGVH (SEQ ID NO: 30), or MLLLVTSLLLCELPHPAFLLIP (SEQ ID NO: 36) or an equivalent of each thereof. In one embodiment, the signal peptide comprises, or consists essentially of, or yet further consists of MYRMQLLSCIALSLALVTNS (SEQ ID NO: 31), or MWLQSLLLLGTVACSIS (SEQ ID NO: 106), or MRISKPHLRSISIQCYLCLLLNSHFLTEA (SEQ ID NO: 107), or MRSSPGNMERIVICLMVIFLGTLV (SEQ ID NO: 108), or an equivalent of each thereof. In another embodiments, the signal peptide comprises, or consists essentially of, or yet further consists of MGWSSIILFLVATATGVH (SEQ ID NO: 30), or MLLLVTSLLLCELPHPAFLLIP (SEQ ID NO: 36) or an equivalent of each thereof. In one embodiment, the signal peptide is a secretary signal.

IL-15 Domain

The nucleic acid molecule also comprises a second nucleic acid molecule that encodes an IL-15 domain that includes at least a functional portion of human IL-15 (e.g., amino acids 30-162 human IL-15 isoform I; GenBank NP_0056) or a functional portion of human IL-15 receptor alpha subunit isoform I (e.g., amino acids 31-205 of GenBank NP_002180) or can be any domain having that structure or function, including but not limited to, soluble IL-15 (sIL-15), membrane bound IL-15 (mbIL-15 or mIL-15), sIL-15 complex IL-15Ra (sIL-15c), and mbIL-15 complexed with IL-15Rα (mbIL-15c or mIL-15c), and mimetics thereof. In some cases, the IL-15 polynucleotide encodes a domain includes a sequence that is at least 90%, at least 95%, at least 98% identical to or identical to:

GIHVFILGCFSAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTS (soluble IL-15; SEQ ID NO: 43). In some cases, the polynucleotide encodes an IL-15 domain has 1, 2, 3, 4 of 5 amino acid changes (preferably conservative) compared to SEQ ID NO: 43. In some embodiments, the polynucleotide encoding the IL-15 domain is codon optimized.

In some embodiments, the polynucleotide encodes an IL-15 domain that includes a transmembrane domain that is at least 90%, at least 95%, at least 98% identical to or identical to: VAISTSTVLLCGLSAVSLLACYL (SEQ ID NO: 74). In some cases, the transmembrane domain within the IL-15 domain has 1, 2, 3, 4 of 5 amino acid changes (preferably conservative) compared to SEQ ID NO: 74.

In some embodiments, the polynucleotide encodes a IL-15 domain includes a transmembrane domain sequence and a soluble IL-15 domain and has a sequence that is at least 90%, at least 95%, at least 98% identical to or identical to: VAISTSTVLLCGL SAVSLLACYLGIHVFILGCFSAGLPKTEANWVNVISDLKKIEDLIQSMHI DATLYTESDVHPSCKVTAMKCFLLELQVISLESGDASIHDTVENLIILANNSL SSNGNVTES GCKECEELEEKNIKEFLQSFVHIVQMFINTS (SEQ ID NO: 77) In some cases, polynucleotide encodes the transmembrane domain within the IL-15 domain has 1, 2, 3, 4 of 5 amino acid changes (preferably conservative) compared to SEQ ID NO: 77.

In some embodiments, the polynucleotide encodes an IL-15 domain that includes a functional portion of IL-15Rα. In some embodiments, the polynucleotide encodes an IL-15Rα includes a sequence that is at least 90%, at least 95%, at least 98% identical to or identical to: ITCPPPMSVEHADIWVKSYSLYSRERYICNSGFKRKAGTSSLTECVLNKATNVAHWTTPSL KCIRDPALVHQRPAPPSTVTTAGVTPQPESL SPSGKEPAASSPSSNNTAATTAAIVPGSQLM PSKSPSTGTTEISSHESSHGTPSQTTAKNWELTASASHQPPGVYPQGHSDTT (amino acids 31-205 of IL-15Ra; SEQ ID NO: 72). In some embodiments, the polynucleotide encodes an IL-15Rα has 1, 2, 3, 4 of 5 amino acid changes (preferably conservative) compared to SEQ ID NO: 72.

In some embodiments, the polynucleotide encodes a IL-15 domain that comprises a linker between the IL-15 portion and the IL-15Rα portion. Any linker disclosed herein may be used in the IL-15 domain. In some embodiments, the polynucleotide encodes a linker that includes a sequence that is at least 90%, at least 95%, at least 98% identical to or identical to:

(SEQ ID NO: 81)
SGGGSGGGGSGGGGSGGGGSGGGS.

In some embodiments, the polynucleotide encodes an IL-15 domain that comprises soluble IL-15, a linker and a portion of IL-15Ra. For example, polynucleotide encodes a sequence that is at least 90%, at least 95%, at least 98% identical to or identical to:

GIHVFILGCFSAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTSSGGGSGGGGSGGGGSGGGGSGGGSLQITCPPPMSVEHADIWVKSYSLYSRERYI CNSGFKRKAGTSSLTECVLNKATNVAHWTTPSLKCIRDPALVHQRPAPPSTVTTAGVTPQP ESL SPSGKEPAASSPSSNNTAATTAAIVPGSQLMPSKSPSTGTTEISSHESSHGTPSQTTAKN WELTASASHQPPGVYPQGHSDTT (SEQ ID NO: 70) or GIHVFILGCFSAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTSSGGGSGGGGSGGGGSGGGGSGGGSITCPPPMSVEHADIWVKSYSLYSRERYICN SGFKRKAGTSSLTECVLNKATNVAHWTTPSLKCIRDPALVHQRPAPPSTVTTAGVTPQPES L SPSGKEPAASSPSSNNTAATTAAIVPGSQLMPSKSPSTGTTEISSHESSHGTPSQTTAKNWE LTASASHQPPGVYPQGHSDTT (SEQ ID NO: 75). In some cases, the polynucleotide encodes an IL-15 domain has 1, 2, 3, 4 of 5 amino acid changes (preferably conservative) compared to SEQ ID NO: 75.

In some embodiments, the polynucleotide encodes an IL-15 domain comprises: a transmembrane domain, soluble IL-15, a linker and a portion of IL-15Ra. For example, a sequence that is at least 90%, at least 95%, at least 98% identical to or identical to:

VAISTSTVLLCGL SAVSLLACYLITCPPPMSVEHADIWVKSYSLYSRERYICNSGFKRKAGT SSLTECVLNKATNVAHWTTPSLKCIRDPALVHQRPAPPSTVTTAGVTPQPESLSPSGKEPAA SSPSSNNTAATTAAIVPGSQLMPSKSPSTGTTEISSHESSHGTPSQTTAKNWELTASASHQPP GVYPQGHSDTTVAISTSTVLLCGLSAVSLLACYLGIHVFILGCFSAGLPKTEANWVNVISDL KKIEDLIQSMHIDATLYTESDVHPSCKVTAMKCFLLELQVISLESGDASIHDTVENLIILANN SLSSNGNVTESGCKECEELEEKNIKEFLQSFVHIVQMFINTS (SEQ ID NO: 76). In some cases, the IL-15 domain has 1, 2, 3, 4 of 5 amino acid changes (preferably conservative) compared to SEQ ID NO: 76.

In some embodiments, the polynucleotide encodes an IL-15 domain that comprises IL-15-linker-IL15Rα. In some embodiments, the IL-15 is N terminal to the IL-15Ra. In some embodiments, the polynucleotide encodes an IL-15 domain that comprises an IL-15Rα-linker-IL15. In some embodiments, the IL-15Rα is N terminal to the IL-15. In some embodiments, the IL-15 domain can comprise SEQ ID NO: 43 with up to 1, 2, 3, 4 or 5 amino acid changes (preferably conservative amino acid changes) or SEQ ID NO: 70 with up to 1, 2, 3, 4 or 5 amino acid changes (preferably conservative amino acid changes) and SEQ ID NO: 72 with up to 1, 2, 3, 4 or 5 amino acid changes (preferably conservative amino acid changes) joined by a flexible linker. In some embodiments, the polynucleotide encodes an IL-15 domain that can comprise SEQ ID NO: 70 with up to 1, 2, 3, 4 or 5 amino acid changes (preferably conservative amino acid changes) and SEQ ID NO: with up to 1, 2, 3, 4 or 5 amino acid changes (preferably conservative amino acid changes) joined by a flexible linker. In some embodiments, polynucleotide encodes the IL-15 domain can comprise SEQ ID NO: with up to 1, 2, 3, 4 or 5 amino acid changes (preferably conservative amino acid changes) and SEQ ID NO: 72 with up to 1, 2, 3, 4 or 5 amino acid changes (preferably conservative amino acid changes) joined by a flexible linker.

In some embodiments, polynucleotide encodes a transmembrane domain “TM” that precedes the IL-15 domain. In some embodiments, the polynucleotide encodes a TM domain is immediately N-terminal to the IL-15 domain. In some embodiments, the polynucleotide encodes a TM domain that comprises the amino acid sequence that is at least 90%, at least 95%, at least 98% identical to or identical to: VAISTSTVLLCGLSAVSLLACYL (SEQ ID NO: 74). In some embodiments, the polynucleotide encodes a TM can comprise SEQ ID NO: 74 with up to 1, 2, 3, 4, or 5 amino acid changes (preferably conservative amino acid changes).

Truncated EGFR or truncated CD19 or LNGFR, and an optional Ribosomal Skip Sequence

In some cases, the nucleic acid molecules encoding the PSCA CAR or PSCA polypeptide are produced using a vector in which the CAR open reading frame is followed by a ribosome skip sequence and a truncated EGFR (EGFRt), which lacks the cytoplasmic signaling tail, or a truncated CD19R or a LNGFR. In this arrangement, co-expression of EGFRt provides an inert, non-immunogenic surface marker that allows for accurate measurement of gene modified cells, and enables positive selection of gene-modified cells, as well as efficient cell tracking of the therapeutic NK cells in vivo following adoptive transfer. Efficiently controlling proliferation to avoid cytokine storm and off-target toxicity is an important hurdle for the success of NK cell immunotherapy. The EGFRt, CD19t, or LNGFR incorporated in the PSCA CAR lentiviral or retroviral vector can act as suicide gene to ablate the CAR+NK cells in cases of treatment-related toxicity.

In some embodiments, the second nucleic acid molecule further comprises a polynucleotide encoding ribosomal skip sequence (e.g., LEGGGEGRGSLLTCGDVEENPGPR; SEQ ID NO: 27) and a polynucleotide encoding a truncated EGFR having a sequence that is at least 90%, at least 95%, at least 98% identical to or identical to:

LVTSLLLCELPHPAFLLIPRKVCNGIGIGEFKDSL SINATNIKHFKNCTSISGDLHILPVAFRG DSF THTPPLDPQELDILKTVKEITGFLLIQAWPENRTDLHAFENLEIIRGRTKQHGQFSLAVV SLNITSLGLRSLKEISDGDVIISGNKNLCYANTINWKKLFGTSGQKTKIISNRGENSCKATGQ VCHALCSPEGCWGPEPRDCVSCRNVSRGRECVDKCNLLEGEPREFVENSECIQCHPECLPQ AMNITCTGRGPDNCIQCAHYIDGPHCVKTCPAGVMGENNTLVWKYADAGHVCHLCHPN CTYGCTGPGLEGCPTNGPKIPSIATGMVGALLLLLVVALGIGLFM (SEQ ID NO: 28). In some cases, the truncated EGFR has 1, 2, 3, 4 of 5 amino acid changes (preferably conservative) compared to SEQ ID NO: 28. Other ribosomal skip sequences useful in the second nucleic acid molecule include T2At having a sequence that is at least 95% identical or identical to: EGRGSLLTCGDVEENPGP (SEQ ID NO: 46) or P2A having a sequence that is at least 95% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NOS: 46 or 47.

In some embodiments, the second nucleic acid molecule can encode a ribosomal skip sequence (e.g., LEGGGEGRGSLLTCGDVEENPGPR; SEQ ID NO: 27) and a polynucleotide encoding truncated CD19R (also called CD19t) having a sequence that is at least 90%, at least 95%, at least 98% identical to or identical to:

MPPPRLLFFLLFLTPMEVRPEEPLVVKVEEGDNAVLQCLKGTSDGPTQQLTWSRESPLKPF LKLSLGLPGLGIHMRPLAIWLFIFNVSQQMGGFYLCQPGPPSEKAWQPGWTVNVEGSGELF RWNVSDLGGLGCGLKNRSSEGPSSPSGKLMSPKLYVWAKDRPEIWEGEPPCVPPRDSLNQ SL SQDL TMAPGSTLWLSCGVPPDSVSRGPL SWTHVHPKGPKSLL SLELKDDRPARDMWV METGLLLPRATAQDAGKYYCHRGNLTMSFHLEITARPVLWHWLLRTGGWKVSAVTLAY LIFCLCSLVGILHLQRALVLRRKR (SEQ ID NO: 26). In some cases, the truncated CD19t has 1, 2, 3, 4 of 5 amino acid changes (preferably conservative) compared to SEQ ID NO: 26. Other ribosomal skip sequences useful in the second nucleic acid molecule include T2At having a sequence that is at least 95% identical or identical to: EGRGSLLTCGDVEENPGP (SEQ ID NO: 46) or P2A having a sequence that is at least 95% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NOS: 46 or 47.

In some embodiments, the second nucleic acid molecule encodes a ribosomal skip sequence (e.g., LEGGGEGRGSLLTCGDVEENPGPR; SEQ ID NO: 27) and tEGFR having a sequence that is at least 90%, at least 95%, at least 98% identical to or identical to:

(SEQ ID NO: 45)
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM.

Other ribosomal skip sequences useful in the second nucleic acid molecule include T2At having bGP-42,DNA sequence that is at least 95% identical or identical to: EGRGSLLTCGDVEENPGP (SEQ ID NO: 46) or P2A having a sequence that is at least 95% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NOS: 46 or 47.

In some embodiments, the second nucleic acid molecule further encodes a ribosomal skip sequence and a truncated LNGFR having a sequence that is at least 90%, at least 95%, at least 98% identical to or identical to:

MGAGATGRAMDGPRLLLLLLLGVSLGGAKEACPTGLYTHSGECCKACNLGEGVAQPCGA NQTVCEPCLDSVTFSDVVSATEPCKPCTECVGLQSMSAPCVEADDAVCRCAYGYYQDETT GRCEACRVCEAGSGLVFSCQDKQNTVCEECPDGTYSDEANHVDPCLPCTVCEDTERQLRE CTRWADAECEEIPGRWITRSTPPEGSDSTAPSTQEPEAPPEQDLIASTVAGVVTTVMGSSQP VVTRGTTDNLIPVYCSILAAVVVGLVAYIAFKRWNSCKQNK (SEQ ID NO: 78). In some cases, the truncated LNGFR has 1, 2, 3, 4 of 5 amino acid changes (preferably conservative) compared to SEQ ID NO: 78. Other ribosomal skip sequences useful the include T2At having a sequence that is at least 95% identical or identical to: EGRGSLLTCGDVEENPGP (SEQ ID NO: 46) or P2A having a sequence that is at least 95% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NOS: 46 or 47.

Vectors

Additionally, provided is a vector comprising, or consisting essentially of, or yet further consisting of one or more of a polynucleotide as disclosed herein, a complement thereof, a reverse sequence thereof, or a reverse-complement thereof.

In some embodiments, the vector is a non-viral vector or a viral vector. In one embodiment, the non-viral vector is a plasmid, e.g., RD114, RD114-TR or VSVG. In one embodiment, the viral vector is selected from the group of a retroviral vector, a lentiviral vector, an adenoviral vector, an adeno-associated viral vector, or an Herpes viral vector.

Additionally, the vector can further comprise a regulatory sequence directing the replication or the expression of the polynucleotide or both. In some embodiment, a plasmid is constructed and prepared, for example as shown in the Example, to produce a viral vector, such as a lentiviral vector.

In some embodiments, a vector is used to replicate or amplify a polynucleotide as disclosed herein. In further embodiments, the vector comprises one or more of: the polynucleotide, a complement thereof, a reverse sequence thereof, or a reverse-complement thereof. In yet further embodiments, the vector further comprises a regulatory sequence directing the replication of the polynucleotide.

In some embodiments, a vector is used to transcript or translate or both transcript and translate a polynucleotide as disclosed herein, for example, to a polypeptide as disclosed herein or a fragment thereof. In further embodiments, the vector comprises one or more of: the polynucleotide, a complement thereof, a reverse sequence thereof, or a reverse-complement thereof. In yet further embodiments, the vector further comprises a regulatory sequence directing the expression of the polynucleotide. Additionally or alternatively, the vector further comprises a ribosome skip sequence that follows the open reading frame of the CAR.

CAR Polypeptides

Also provided are the CAR and IL-15 polypeptides encoded by the nucleic acid molecules described herein.

In various embodiments: the chimeric antigen receptor or polypeptide comprises: a PSCA scFv (A1 LH), e.g., an scFv comprising the amino acid sequence

DIQLTQSPSTLSASVGDRVTITCSASSSVRFIHWYQQKPGKAPKRLIYDTSKLASGVPSRFSG SGSGTDFTLTISSLQPEDFATYYCQQWGSSPFTFGQGTKVEIKGSTSGGGGSGGGGSGGGG SEVQLVEYGGGLVQPGGSLRLSCAASGFNIKDYYIHWVRQAPGKGLEWVAWIDPENGDT EFVPKFQGRATMSADTSKNTAYLQMNSLRAEDTAVYYCKTGGFWGQGTLVTVSS (SEQ ID NO: 1) with up to 5 or up to 10 single amino acid substitutions (preferably outside the CDRs).

In various embodiments: the chimeric antigen receptor or polypeptide comprises: a PSCA scFv (A1 HL), e.g., an scFv comprising the amino acid sequence

EVQLVEYGGGLVQPGGSLRLSCAASGFNIKDYYIHWVRQAPGKGLEWVAWIDPENGDTE FVPKFQGRATMSADTSKNTAYLQMNSLRAEDTAVYYCKTGGFWGQGTLVTVSSGGGGS GGGGSGGGGSDIQLTQSPSTLSASVGDRVTITCSASSSVRFIHWYQQKPGKAPKRLIYDTSK LASGVPSRFSGSGSGTDFTLTISSLQPEDFATYYCQQWGSSPFTFGQGTKVEIKGSTS (SEQ ID NO:40) with up to 5 or up to 10 single amino acid substitutions (preferably outside the CDRs).

In certain embodiments, the PSCA scFv comprises a light chain variable region (A1 VL) that is at least 95% identical to or includes up to 5 single amino acid substitutions (preferably outside the CDRs, underlined) compared to:

(SEQ ID NO: 32)
DIQLTQSPSTLSASVGDRVTITCSASSSVRFIHWYQQKPGKAPKRLIY
DTSKLASGVPSRFSGSGSGTDFTLTISSLQPEDFATYYCQQWGSSPFT
FGQGTKVEIKGSTS.

In certain embodiments, the PSCA scFv comprises a heavy chain variable region (A1 VH) that is at least 95% identical to or includes up to 5 single amino acid substitutions (preferably outside the CDRs, underlined) compared to:

(SEQ ID NO: 33)
EVQLVEYGGGLVQPGGSLRLSCAASGFNIKDYYIHWVRQAPGKGLEWVA
WIDPENGDTEFVPKFQGRATMSADTSKNTAYLQMNSLRAEDTAVYYCKT
GGFWGQGTLVTVSS.

In some embodiments, the PSCA scFv comprises a light chain variable region (M1 VL) that is at least 95% identical to or includes up to 5 single amino acid substitutions (preferably outside the CDRs, underlined) compared to:

(SEQ ID NO: 34)
DIQMTQSPSSLSASVGDRVTITCGTSQDINNYLNWYQQKPGKVPKLLIY
YTSRLHSGVPSRFSGSGSGTDFTLTISSLQPEDVATYYCQQSKTLPWT
FGGGTQLTVL.

In certain embodiments, the PSCA scFv comprises a heavy chain variable region (M1 VH) that is at least 95% identical to or includes up to 5 single amino acid substitutions (preferably outside the CDRs, underlined) compared to:

(SEQ ID NO: 35)
QVQLVESGGGLVKPGGSLRLSCAASGFTFSSYSMSWIRQAPGKGLEWVS
YINDSGGSTFYPDTVKGRFTISRDNAKNSLYLQMNSLRAEDTAVYYCAR
RMYYGNSHWHFDVWGQGTTVTVSS.

In various embodiments: the chimeric antigen receptor or polypeptide comprises: a PSCA scFv, e.g., an scFv (M1 LH) comprising the amino acid sequence

DIQMTQSPSSLSASVGDRVTITCGTSQDINNYLNWYQQKPGKVPKLLIYYTSRLHSGVPSRF SGSGSGTDFTLTISSLQPEDVATYYCQQSKTLPWTFGGGTQLTVLGGGGSGGGGSGGGGS QVQLVESGGGLVKPGGSLRLSCAASGFTFSSYSMSWIRQAPGKGLEWVSYINDSGGSTFYP DTVKGRFTISRDNAKNSLYLQMNSLRAEDTAVYYCARRMYYGNSHWHFDVWGQGTTVT VSS (SEQ ID NO:41) with up to 5 or up to 10 single amino acid substitutions (preferably outside the CDRs).

In various embodiments: the chimeric antigen receptor or polypeptide comprises: a PSCA scFv, e.g., an scFv (M1 HL) comprising the amino acid sequence

QVQLVESGGGLVKPGGSLRLSCAASGFTFSSYSMSWIRQAPGKGLEWVSYINDSGGSTFYP DTVKGRFTISRDNAKNSLYLQMNSLRAEDTAVYYCARRMYYGNSHWHFDVWGQGTTVT VSSGGGGSGGGGSGGGGSDIQMTQSPSSLSASVGDRVTITCGTSQDINNYLNWYQQKPGK VPKLLIYYTSRLHSGVPSRFSGSGSGTDFTLTISSLQPEDVATYYCQQSKTLPWTFGGGTQL TVL (SEQ ID NO:42) with up to 5 or up to 10 single amino acid substitutions (preferably outside the CDRs).

The PSCA targeted CAR (also called “PSCA CAR”) or PSCA targeted polypeptide (also called “PSCA polypeptide”) described herein include a PSCA targeting scFv, e.g., a PSCA scFv described above. In some embodiments, an scFv comprising the amino acid sequence:

DIQLTQSPSTLSASVGDRVTITCSASSSVRFIHWYQQKPGKAPKRLIYDTSKLASGVPSRFSG SGSGTDFTLTISSLQPEDFATYYCQQWGSSPFTFGQGTKVEIKGSTS (SEQ ID NO:32) and the sequence EVQLVEYGGGLVQPGGSLRLSCAASGFNIKDYYIHWVRQAPGKGLEWVAWIDPENGDTE FVPKFQGRATMSADTSKNTAYLQMNSLRAEDTAVYYCKTGGFWGQGTLVTVSS (SEQ ID NO:33) (in either order) joined by a flexible linker.

In some embodiments, an scFv comprising the amino acid sequence: DIQMTQSPSSLSASVGDRVTITCGTSQDINNYLNWYQQKPGKVPKLLIYYTSRLHSGVPSRF SGSGSGTDFTLTISSLQPEDVATYYCQQSKTLPWTFGGGTQLTVL (SEQ ID NO:34) and the sequence QVQLVESGGGLVKPGGSLRLSCAASGFTFSSYSMSWIRQAPGKGLEWVSYINDSGGSTFYP DTVKGRFTISRDNAKNSLYLQMNSLRAEDTAVYYCARRMYYGNSHWHFDVWGQGTTVT VSS (SEQ ID NO:35) (in either order) joined by a flexible linker.

In some embodiments, a useful flexible linker is 1, 2, 3, 4, 5, 6, 7, 8, 9, or 10 repeats of the sequence GGGS (SEQ ID NO:38, repeats disclosed as SEQ ID NO: 111). In some embodiments, a useful flexible linker is 1, 2, 3, 4, 5, 6, 7, 8, 9, or 10 repeats of the sequence GGGGS (SEQ ID NO:39, repeats disclosed as SEQ ID NO: 112). A useful linker could comprise (G4S)₃

(SEQ ID NO: 37)
GGGGSGGGGSGGGGS.

A useful PSCA CAR or PSCA polypeptide can consist of or comprises the amino acid sequence of SEQ ID NO: X or SEQ ID NO: Y (mature CAR lacking a signal sequence). Any disclosed CAR or polypeptide can be expressed in a form that includes a signal sequence, e.g., a human GM-CSF receptor alpha signal sequence (MLLLVTSLLLCELPHPAFLLIP; SEQ ID NO:36); a IgGk signal peptide (METDTLLLWVLLLWVPGSTG; SEQ ID NO:29); a IgG2 signal peptide (MGWSSIILFLVATATGVH; SEQ ID NO:30); a IL-2 signal peptide

(MYRMQLLSCIALSLALVTNS; SEQ ID NO: 31).

The CAR or polypeptide can be expressed with additional sequences that are useful for monitoring expression, for example, a T2A or P2A skip sequence and a truncated EGFR or truncated CD19 or LNGFR (can consist of or comprise the amino acid sequence of SEQ ID NO:31).

The CAR or polypeptide can comprise the amino acid sequence of SEQ ID NOs: 1 or 40-42 or can comprise an amino acid sequence that is at least 95%, 96%, 97%, 98% or 99% identical to SEQ ID NOs: 1 or 40-42. The CAR or polypeptide can comprise the amino acid sequence of any of SEQ ID NOs: 1 or 40-42 with up to 1, 2, 3, 4 or 5 amino acid changes (preferably conservative amino acid changes). The CAR or polypeptide can comprise SEQ ID NO:32 with up to 1, 2, 3, 4 or 5 amino acid changes (preferably conservative amino acid changes) and SEQ ID NO: 33 with up to 1, 2, 3, 4 or 5 amino acid changes (preferably conservative amino acid changes) joined by a flexible linker. The CAR or polypeptide can comprise SEQ ID NO: 34 with up to 1, 2, 3, 4 or 5 amino acid changes (preferably conservative amino acid changes) and SEQ ID NO: 35 with up to 1, 2, 3, 4 or 5 amino acid changes (preferably conservative amino acid changes) joined by a flexible linker.

In some embodiments, the nucleic acid encoding amino acid sequences SEQ ID NOs: 1, 32-35, and 40-42 are codon optimized.

The CAR or polypeptide described herein can include a spacer located between the PSCA targeting domain (i.e., a PSCA targeted ScFv or variant thereof) and the transmembrane domain. A variety of different spacers can be used. Some of them include at least portion of a human Fc region, for example a hinge portion of a human Fc region or a CH3 domain or variants thereof. Table 1 provides various spacers that can be used in the CARs described herein.

IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain.

The immunoglobulin derived sequences can include one or more amino acid modifications, for example, 1, 2, 3, 4 or 5 substitutions, e.g., substitutions that reduce off-target binding.

The spacer region can also comprise an IgG4 hinge region having the sequence ESKYGPPCPSCP (SEQ ID NO: 4) or ESKYGPPCPPCP (SEQ ID NO: 3). The spacer region can also comprise the hinge sequence ESKYGPPCPPCP (SEQ ID NO: 3) followed by the linker sequence GGGSSGGGSG (SEQ ID NO: 2) followed by IgG4 CH3 sequence

(SEQ ID NO: 12)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

Thus, the entire spacer region can comprise the sequence:

(SEQ ID NO: 79)
ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A variety of transmembrane domains can be used in the CAR. In some cases, the transmembrane domain is a CD28 transmembrane domain that includes a sequence that is at least 90%, at least 95%, at least 98% identical to or identical to:

FWVLVVVGGVLACYSLLVTVAFIIFWV (SEQ ID NO: 14). In some cases, the CD28 transmembrane domain has 1, 2, 3, 4 of 5 amino acid changes (preferably conservative) compared to SEQ ID NO:14. Table 2 includes examples of suitable transmembrane domains. Where a spacer region is present, the transmembrane domain (TM) is located carboxy terminal to the spacer region.

The costimulatory domain can be any domain that is suitable for use with a CD3ξ signaling domain. In some cases, the co-signaling domain is a CD28 co-signaling domain that includes a sequence that is at least 90%, at least 95%, at least 98% identical to or identical to: RSKRSRLLHSDYMNMTPRRPGPTRKHYQPYAPPRDFAAYRS (SEQ ID NO: 22). In some cases, the 4-1BB co-signaling domain has 1, 2, 3, 4 of 5 amino acid changes (preferably conservative) compared to SEQ ID NO: 22.

The costimulatory domain(s) are located between the transmembrane domain and the CD3(signaling domain. Table 3 includes examples of suitable costimulatory domains together with the sequence of the CD3ξ signaling domain.

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ξ signaling domain and a short linker consisting of 2-10, e.g., 3 amino acids (e.g., GGG) is can be positioned between the costimulatory domain and the CD3ξ signaling domain.

The CD3ξ signaling domain can be any domain that is suitable for use with a CD3(signaling domain. In some cases, the CD3ξ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEGLY NELQKDKMAEAYSEIGMKGERRRGKGHDGLYQGLSTATKDTYDALHMQALPPR (SEQ ID NO: 21). In some cases, the CD3ξ signaling has 1, 2, 3, 4 of 5 amino acid changes (preferably conservative) compared to SEQ ID NO: 21.

The IL-15 domain is a domain that includes at least a functional portion of human IL-15 (e.g., amino acids 30-162 human IL-15 isoform I; GenBank NP_0056) or a functional portion of human IL-15 receptor alpha subunit isoform I (e.g., amino acids 31-205 of GenBank NP_002180) or can be any domain having that structure or function, including but not limited to, soluble IL-15 (sIL-15), membrane bound IL-15 (mbIL-15 or mIL-15), sIL-15 complex IL-15Rα (sIL-15c), and mbIL-15 complexed with IL-15Rα (mbIL-15c or mIL-15c), and mimetics thereof. In some cases, the IL-15 domain includes a sequence that is at least 90%, at least 95%, at least 98% identical to or identical to:

GIHVFILGCFSAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTS (soluble IL-15; SEQ ID NO: 43). In some cases, the IL-15 domain has 1, 2, 3, 4 of 5 amino acid changes (preferably conservative) compared to SEQ ID NO: 43. In some embodiments, the IL-15 domain is codon optimized.

In some embodiments, the IL-15 domain includes a transmembrane domain that is at least 90%, at least 95%, at least 98% identical to or identical to: VAISTSTVLLCGLSAVSLLACYL (SEQ ID NO: 74) In some cases, the transmembrane domain within the IL-15 domain has 1, 2, 3, 4 of 5 amino acid changes (preferably conservative) compared to SEQ ID NO: 74.

In some embodiments, the IL-15 domain includes a transmembrane domain sequence and a soluble IL-15 domain and has a sequence that is at least 90%, at least 95%, at least 98% identical to or identical to:

VAISTSTVLLCGL SAVSLLACYLGIHVFILGCFSAGLPKTEANWVNVISDLKKIEDLIQSMHI DATLYTESDVHPSCKVTAMKCFLLELQVISLESGDASIHDTVENLIILANNSL SSNGNVTES GCKECEELEEKNIKEFLQSFVHIVQMFINTS (SEQ ID NO: 77) In some cases, the transmembrane domain within the IL-15 domain has 1, 2, 3, 4 of 5 amino acid changes (preferably conservative) compared to SEQ ID NO: 77.

In some embodiments, the IL-15 domain comprises a linker between the IL-15 portion and the IL-15Rα portion. Any linker disclosed herein may be used in the IL-15 domain. In some embodiments, the linker includes a sequence that is at least 90%, at least 95%, at least 98% identical to or identical to: SGGGSGGGGSGGGGSGGGGSGGGS (SEQ ID NO: 81).

In some embodiments, the IL-15 domain includes a functional portion of IL-15Rα. In some embodiments, the IL-15Rα includes a sequence that is at least 90%, at least 95%, at least 98% identical to or identical to:

ITCPPPMSVEHADIWVKSYSLYSRERYICNSGFKRKAGTSSLTECVLNKATNVAHWTTPSL KCIRDPALVHQRPAPPSTVTTAGVTPQPESL SPSGKEPAASSPSSNNTAATTAAIVPGSQLM PSKSPSTGTTEISSHESSHGTPSQTTAKNWELTASASHQPPGVYPQGHSDTT (amino acids 31-205 of IL-15Ra; SEQ ID NO: 72). In some embodiments, the IL-15Rα has 1, 2, 3, 4 of 5 amino acid changes (preferably conservative) compared to SEQ ID NO: 72.

In some embodiments, the IL-15 domain comprises soluble IL-15, a linker and a portion of IL-15Ra. For example, a sequence that is at least 90%, at least 95%, at least 98% identical to or identical to:

GIHVFILGCF SAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTSSGGGSGGGGSGGGGSGGGGSGGGSLQITCPPPMSVEHADIWVKSYSLYSRERYI CNSGFKRKAGT SSLTECVLNKATNVAHWTTPSLKCIRDPALVHQRPAPPSTVTTAGVTPQP ESL SPSGKEPAASSPSSNNTAATTAAIVPGSQLMPSKSPSTGTTEISSHESSHGTPSQTTAKN WELTASASHQPPGVYPQGHSDTT (SEQ ID NO: 70) or GIHVFILGCFSAGLPKTEANWVNVISDLKKIEDLIQ SMHIDATLYTESDVHPSCKVTAMKCF LLELQVISLESGDASIHDTVENLIILANNSL SSNGNVTESGCKECEELEEKNIKEFLQSFVHIV QMFINTSSGGGSGGGGSGGGGSGGGGSGGGSITCPPPMSVEHADIWVKSYSLYSRERYICN SGFKRKAGTSSLTECVLNKATNVAHWTTPSLKCIRDPALVHQRPAPPSTVTTAGVTPQPES L SPSGKEPAASSPSSNNTAATTAAIVPGSQLMPSKSPSTGTTEISSHESSHGTPSQTTAKNWE LTASASHQPPGVYPQGHSDTT (SEQ ID NO: 75). In some cases, the IL-15 domain has 1, 2, 3, 4 of 5 amino acid changes (preferably conservative) compared to SEQ ID NO: 70.

In some embodiments, the IL-15 domain comprises: a transmembrane domain, soluble IL-15, a linker and a portion of IL-15Ra. For example, a sequence that is at least 90%, at least 95%, at least 98% identical to or identical to:

VAISTSTVLLCGL SAVSLLACYLITCPPPMSVEHADIWVKSYSLYSRERYICNSGFKRKAGT SSLTECVLNKATNVAHWTTPSLKCIRDPALVHQRPAPPSTVTTAGVTPQPESLSPSGKEPAA SSPSSNNTAATTAAIVPGSQLMPSKSPSTGTTEISSHESSHGTPSQTTAKNWELTASASHQPP GVYPQGHSDTTVAISTSTVLLCGLSAVSLLACYLGIHVFILGCFSAGLPKTEANWVNVISDL KKIEDLIQSMHIDATLYTESDVHPSCKVTAMKCFLLELQVISLESGDASIHDTVENLIILANN SLSSNGNVTESGCKECEELEEKNIKEFLQSFVHIVQMFINTS (SEQ ID NO: 76). In some cases, the IL-15 domain has 1, 2, 3, 4 of 5 amino acid changes (preferably conservative) compared to SEQ ID NO: 76.

In some embodiments, the IL-15 domain comprises IL-15-linker-IL15Rα. In some embodiments, the IL-15 is N terminal to the IL-15Rα. In some embodiments, the IL-15 domain comprises an IL-15Rα-linker-IL15. In some embodiments, the IL-15Rα is N terminal to the IL-15.

In some embodiments, the IL-15 domain can comprise SEQ ID NO: 43 with up to 1, 2, 3, 4 or 5 amino acid changes (preferably conservative amino acid changes) or SEQ ID NO: 70 with up to 1, 2, 3, 4 or 5 amino acid changes (preferably conservative amino acid changes) and SEQ ID NO: 72 with up to 1, 2, 3, 4 or 5 amino acid changes (preferably conservative amino acid changes) joined by a flexible linker. In some embodiments, the IL-15 domain can comprise SEQ ID NO: 43 with up to 1, 2, 3, 4 or 5 amino acid changes (preferably conservative amino acid changes) and SEQ ID NO: 72 with up to 1, 2, 3, 4 or 5 amino acid changes (preferably conservative amino acid changes) joined by a flexible linker. In some embodiments, the IL-15 domain can comprise SEQ ID NO: 70 with up to 1, 2, 3, 4 or 5 amino acid changes (preferably conservative amino acid changes) and SEQ ID NO: 72 with up to 1, 2, 3, 4 or 5 amino acid changes (preferably conservative amino acid changes) joined by a flexible linker.

In some embodiments, a transmembrane domain precedes the IL-15 domain. In some embodiments, the TM domain is immediately N-terminal to the IL-15 domain. In some embodiments, the TM domain comprises the amino acid sequence that is at least 90%, at least 95%, at least 98% identical to or identical to: VAISTSTVLLCGLSAVSLLACYL (SEQ ID NO: 74). In some embodiments, the TM can comprise SEQ ID NO: 74 with up to 1, 2, 3, 4, or 5 amino acid changes (preferably conservative amino acid changes).

In some embodiments, a CAR or peptide described herein can comprise a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to:

LVTSLLLCELPHPAFLLIPRKVCNGIGIGEFKDSL SINATNIKHFKNCTSISGDLHILPVAFRG DSF THTPPLDPQELDILKTVKEITGFLLIQAWPENRTDLHAFENLEIIRGRTKQHGQFSLAVV SLNITSLGLRSLKEISDGDVIISGNKNLCYANTINWKKLFGTSGQKTKIISNRGENSCKATGQ VCHALCSPEGCWGPEPRDCVSCRNVSRGRECVDKCNLLEGEPREFVENSECIQCHPECLPQ AMNITCTGRGPDNCIQCAHYIDGPHCVKTCPAGVMGENNTLVWKYADAGHVCHLCHPN CTYGCTGPGLEGCPTNGPKIPSIATGMVGALLLLLVVALGIGLFM (SEQ ID NO: 28). In some cases, the truncated EGFR has 1, 2, 3, 4 of 5 amino acid changes (preferably conservative) compared to SEQ ID NO: 28.

In some embodiments, a CAR or peptide described herein can comprise a ribosomal skip sequence (e.g., LEGGGEGRGSLLTCGDVEENPGPR; SEQ ID NO: 27) and a truncated CD19R (also called CD19t) having a sequence that is at least 90%, at least 95%, at least 98% identical to or identical to:

MPPPRLLFFLLFLTPMEVRPEEPLVVKVEEGDNAVLQCLKGTSDGPTQQLTWSRESPLKPF LKLSLGLPGLGIHMRPLAIWLFIFNVSQQMGGFYLCQPGPPSEKAWQPGWTVNVEGSGELF RWNVSDLGGLGCGLKNRSSEGPSSPSGKLMSPKLYVWAKDRPEIWEGEPPCVPPRDSLNQ SL SQDL TMAPGSTLWLSCGVPPDSVSRGPL SWTHVHPKGPKSLL SLELKDDRPARDMWV METGLLLPRATAQDAGKYYCHRGNLTMSFHLEITARPVLWHWLLRTGGWKVSAVTLAY LIFCLCSLVGILHLQRALVLRRKR (SEQ ID NO: 26). In some cases, the truncated CD19t has 1, 2, 3, 4 of 5 amino acid changes (preferably conservative) compared to SEQ ID NO: 26.

In some embodiments, a CAR or peptide described herein can comprise a ribosomal skip sequence (e.g., LEGGGEGRGSLLTCGDVEENPGPR; SEQ ID NO: 27) and tEGFR having a sequence that is at least 90%, at least 95%, at least 98% identical to or identical to:

(SEQ ID NO: 45)
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM.

In some embodiments, a CAR or peptide described herein can comprise a ribosomal skip sequence and a truncated LNGFR having a sequence that is at least 90%, at least 95%, at least 98% identical to or identical to:

MGAGATGRAMDGPRLLLLLLLGVSLGGAKEACPTGLYTHSGECCKACNLGEGVAQPCGA NQTVCEPCLDSVTFSDVVSATEPCKPCTECVGLQSMSAPCVEADDAVCRCAYGYYQDETT GRCEACRVCEAGSGLVFSCQDKQNTVCEECPDGTYSDEANHVDPCLPCTVCEDTERQLRE CTRWADAECEEIPGRWITRSTPPEGSDSTAPSTQEPEAPPEQDLIASTVAGVVTTVMGSSQP VVTRGTTDNLIPVYCSILAAVVVGLVAYIAFKRWNSCKQNK (SEQ ID NO: 78). In some cases, the truncated LNGFR has 1, 2, 3, 4 of 5 amino acid changes (preferably conservative) compared to SEQ ID NO: 78.

Other ribosomal skip sequences useful in a CAR or peptide described herein include T2At having a sequence that is at least 95% identical to: EGRGSLLTCGDVEENPGP (SEQ ID NO: 46) or P2A having a sequence that is at least 95% identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NO:46 or 47.

Cells

Further provided is an isolated cell comprising one or more of the following: a nucleic acid molecule as encoded herein, a polypeptide encoded by the nucleic acid molecules as disclosed herein, a CAR as disclosed herein, a cytokine as disclosed herein, a suicide gene product or a detectable marker or both as disclosed herein, a suicide gene as disclosed herein, a polynucleotide as disclosed herein, a vector as disclosed herein, or a vector system as disclosed herein.

Accordingly, provided is a cell comprising or expressing a polypeptide as described herein, or a polynucleotide encoding the polypeptide, or both. In some embodiments, the polypeptide is further processed, for example, cleaved by itself or by the cell, to yield any one or two or three or all four of the following: the CAR peptide, the cytokine, and the suicide gene product or detectable marker or both. In some embodiments, the cell further comprises any one or two or three or all four of the following: the CAR peptide, the cytokine, and the suicide gene product or detectable marker or both. In some embodiments, the cell expresses the CAR on the cell membrane. In further embodiments, the cell secretes the cytokine out of the cell or expresses the cytokine on the cell membrane. Additionally or alternatively, the cell expresses the suicide gene product or detectable marker or both, for example on the cell membrane. In some embodiments, the cell comprises the polynucleotide as disclosed herein and a regulatory sequence suitable for replicating or expressing the polynucleotide in the cell. In some embodiments, the cell is isolated. In one aspect, the cell comprises a polynucleotide encoding, or the encoded polypeptide comprising, or consisting essentially of, or yet further consisting of one or more of (i) an amino acid sequence of a PSCA CAR as disclosed herein, (ii) an amino acid sequence of an IL-15 domain, or (iii) an amino acid sequence of a suicide gene product or a detectable marker or both as disclosed herein; or a polynucleotide as disclosed herein; or both of the polypeptide and the polynucleotide. In some embodiments, the polypeptide is further processed, for example, cleaved by itself or by the cell, to yield one or more of the CAR peptide, the IL-15 domain, or the suicide gene product or detectable marker or both. In further embodiments, the cell further comprises one or more of the CAR peptide, the IL-15 domain, or the suicide gene product a detectable marker or both. In yet further embodiments, the cell expresses the CAR on the cell membrane. In some embodiments, the cell expresses the IL-15 domain. In further embodiments, the cell secretes the IL-15 domain. In some embodiments, the cell expresses the IL-15 domain on the cell membrane. In some embodiments, the cell expresses the suicide gene product or detectable marker or both. In further embodiments, the cell expresses the suicide gene product or detectable marker or both on the cell membrane. In some embodiments, the cell comprises or further comprises the polynucleotide as disclosed herein and a regulatory sequence suitable for replicating or expressing the polynucleotide in the cell. In some embodiments, the cell is isolated.

In some embodiments, the cell is a prokaryotic cell or a eukaryotic cell. In some embodiments, the cell is a eukaryotic cell, optionally selected from an animal cell, a mammalian cell, a bovine cell, a feline cell, a canine cell, a murine cell, an equine cell, or a human cell. In some embodiments, the eukaryotic cell is an immune cell, optionally a T-cell, a B cell, a NK cell, a NKT cell, a dendritic cell, a myeloid cell, a monocyte, or a macrophage, optionally derived from hematopoietic stem cells (HSCs) or induced pluripotent stem cells (iPSCs). In some embodiments, HSCs and iPSCs are referred to herein as a precursor cell. In some embodiments, the cell is a stem cell, such as an HSC or an iPSC.

In some embodiments, the cell is an immune cell such as a T-cell, a B cell, an NK cell, an NKT cell, a dendritic cell, a myeloid cell, a monocyte, a macrophage, any subsets thereof, or any other immune cell. In further embodiments, the immune cell is derived from hematopoietic stem cells (HSCs) or induced pluripotent stem cells (iPSCs). The eukaryotic cell can be from any preferred species, e.g., an animal cell, a mammalian cell such as a human, a bovine cell, a murine cell, an equine cell, a feline cell, or a canine cell. The cells may be derived from patients, donors, or cell lines, such as those available off-the-shelf. The cells can be autologous or allogeneic to the subject being treated. In some embodiments, the cell further comprise a detectable or purification marker. In some embodiments, the cell expresses a CAR as disclosed herein.

Also provided is a cell population comprising, or alternatively consisting essentially of, or yet consisting of a cell as disclosed herein. In some embodiments, the cell population is homogenous or heterogeneous. In further embodiments, the cell population is substantially homogenous, for example, consisting essentially of the one or more isolated cells. In some embodiments, the cell population is heterogeneous, such as comprising, or consisting essentially of, or yet further consisting of an isolated cell introduced with a polynucleotide as disclosed herein and a residual cell not introduced with the polynucleotide. In some embodiments, the cell is a T cell that has been modified to remove CD52 expression using gene editing technology, e.g., CRISPR or TALEN.

In some embodiments, the cell is for replicating the polynucleotide or the vector or a vector system. In further embodiments, the cell is a cell line. In some embodiments, the cell may be a prokaryotic or a eukaryotic cell. In further embodiments, the cell is an E. coli cell. In yet further embodiments, the cell is a MAX Efficiency™ Stbl2™ Competent Cell (Invitrogen, 10268019) which is for propagating a polynucleotide with a size larger than 10 Kb (such as P64) with optimized efficiency and quality. In some embodiments, the cell is the NEB® Stable Competent E. coli (High Efficiency) (NEB, C3030H), which is suitable for other plasmids with a size equal to or less than 10 Kb.

Further provided is an isolated cell comprising one or more of the following: a polypeptide as disclosed herein, or a fragment thereof as disclosed herein, a CAR as disclosed herein, a polynucleotide as disclosed herein, or a vector as disclosed herein, or a vector system as disclosed herein. In some embodiments, the cell comprises the CAR in the cell membrane. In further embodiments, the cell secretes a cytokine as disclosed herein. Additionally or alternatively, the cell expresses a suicide gene product or a detectable marker or both as disclosed herein, for example, on the cell surface.

Prior to expansion and genetic modification of the cells disclosed herein, cells may be obtained from a subject—for instance, in embodiments involving autologous therapy—or a commercially available cell line or culture, or a stem cell such as an induced pluripotent stem cell (iPSC).

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Production Methods and Compositions

In one aspect, provided is a method of producing a cell as disclosed herein, such as a cell expressing a CAR. In some embodiments, the cell further secretes a cytokine or expresses a suicide gene product (or a detectable marker or both) or both secretes a cytokine and expresses a suicide gene product or a detectable marker or both. In some embodiments, the method comprises, or consists essentially of, or yet further consists of transducing a cell or a population thereof with a polynucleotide as disclosed herein or a vector as disclosed herein or a vector system as disclosed herein. In some embodiments, the method comprises, or consists essentially or, or yet further consists of transducing a cell or a population thereof with the vectors of the vector system concurrently or subsequently. Additionally or alternatively, the method comprises, or consists essentially of, or yet further consists of culturing a cell or a cell population as disclosed herein.

In some embodiments, the cell is selected from a hematopoietic stem cell (HSC), an induced pluripotent stem cell (iPSC), or an immune cell. In some embodiments, the method further comprises culturing the transduced HSC or iPSC under conditions for differentiation to an immune cell. In some embodiments, the immune cell is selected from a T-cell, a B cell, an NK cell, a dendritic cell, a myeloid cell, a monocyte, or a macrophage. In some embodiments, the immune cell is derived from an HSC or an iPSC. In some embodiments, the cell is an immune cell.

Methods of isolating relevant cells are well known in the art and can be readily adapted to the present application; an exemplary method is described in the examples below. Isolation methods for use in relation to this disclosure include, but are not limited to Life Technologies DYNABEADS® System; STEMCELL™ Technologies EASYSEP™, ROBOSEP™ ROSETTESEP™, SEPMATE™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+MicroBeads may be used to isolate CD4+ and CD8+ T-cells. Alternate non-limiting examples of cells that may be isolated according to known techniques include bulked T-cells, NK T-cells, and gamma delta T-cells.

In some embodiments, a method as disclosed herein further comprises isolating cells expressing a suicide gene product as disclosed herein. In further embodiments, the method further comprises contacting cells to be isolated with an antibody that recognizes and binds to a suicide gene product, such as RQR8 (in some embodiments, an anti-RQR8 antibody is an anti-CD34 antibody or an anti-CD20 antibody), thereby isolating the suicide-gene-product-expressing cells. In further embodiments, the anti-CD20 antibody is selected from one or more of the following: rituximab, ocrelizumab, ofatumumab, binutuzumab, ibritumomab, or iodine i 131 tositumomab. Additionally or alternatively, the anti-CD34 antibody is selected from human CD34 APC-conjugated antibody (R&D Systems, Catalog Number FAB7227A), anti-human CD34 antibody, Clone 581 (STEMCELL™ Technologies), or purified anti-human CD34 antibody, Clone 561 (BIOLEGEND©). In further embodiments, an CD34 MicroBead kit is used to isolate the suicide-gene-product-expressing cells, such as CD34 MicroBead Kits (available from Miltenyi Biotec), DYNABEADS™ CD34 Positive Isolation Kit (available from INVITROGEN™), or EASYSEP™ Human CD34 Positive Selection Kit II (available from STEMCELL™ Technologies).

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™), NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and-4, CCRF-HSB-2 (CCL-120.1), J.RT3-T3.5 (ATCC TIB-153), J45.01 (ATCC CRL-1990), J.CaM1.6 (ATCC CRL-2063), RS4;II (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4,-5,-6,-7,-8,-9,-10, and-16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK—YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-I leukemia, KG-I leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-a and TCR-P) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

The preparation of exemplary vectors and the generation of CAR expressing cells using said vectors is discussed herein. In summary, the expression of natural or synthetic nucleic acids encoding a polypeptide as disclosed herein, a CAR as disclosed herein, or any combination thereof is typically achieved by operably linking a nucleic acid encoding the polypeptide, CAR to a promoter, and incorporating the construct into an expression vector. The vectors can be suitable for replication and integration eukaryotes. Methods for producing cells comprising vectors or exogenous nucleic acids are well-known in the art. See, for example, Sambrook et al. (2001, Molecular Cloning: A Laboratory Manual, Cold Spring Harbor Laboratory, New York).

The polynucleotide can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer or promoter or both which directs or direct efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently co-transfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then co-cultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either co-transfection with a selectable marker or infection with pseudotyped virus. In both cases, the vectors integrate. Alternatively, vectors can be introduced in an episomally maintained plasmid. High titer recombinant retrovirus-containing supernatants are produced.

In some embodiments, the cells expressing a CAR or a suicide gene product (or a detectable marker or both) or both are enriched, for example, via selection based on the binding to the antigen that the CAR recognizes and binds (such as a CD19 or a fragment thereof) or the binding to a ligand of the suicide gene product (such as an antibody) or both. Such selection may be achieved by immobilizing the antigen or the ligand or both on a plate, a bead, a column, a membrane, a matrix, or any other suitable solid support, and selecting the cells recognizing and binding the immobilized antigen or ligand or both.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the tumor relevant antigen ex vivo can activate and expand the selected CAR expressing cell subpopulation. Alternatively, the cells may be activated in vivo by interaction with a tumor relevant antigen. Without wishing to be bound by the theory, a cytokine as disclosed herein, such as IL15, expressed by the cell, improves activation and expansion of the cells.

In the case of certain immune cells, additional cell populations, soluble ligands or cytokines or both, or other stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies DYNABEADS© System activation and expansion kits; BD Biosciences PHOSFLOW™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 DYNABEADS© may be used to activate and expand a population of isolated T-cells.

In one aspect, provided is a composition comprising, or consisting essentially of, or yet further consisting of a carrier (optionally a pharmaceutically acceptable carrier) and one or more of the following: a polypeptide as disclosed herein or a fragment thereof, a CAR as disclosed herein, a polynucleotide as disclosed herein, a vector as disclosed herein, a vector system as disclosed herein an isolated complex, an isolated cell as disclosed herein, or a cell population as disclosed herein. In a further embodiment, the carrier is a pharmaceutically acceptable carrier.

In another aspect, provided is an isolated complex comprising, or consisting essentially of, or yet further consisting of one or more of the following: a polypeptide as disclosed herein bound to a cancer cell (for example, expressing PSCA), a CAR as disclosed herein bound to a cancer cell (for example, expressing PSCA), an isolated cell as disclosed herein bound to a cancer cell (for example, expressing PSCA), a cancer cell (for example, expressing PSCA) bound with an isolated or engineered cell, an isolated or engineered cell as disclosed herein bound to a cytokine and a cancer cell (for example, expressing PSCA); or a cancer cell (for example, expressing PSCA) and an isolated or engineered cell as disclosed herein which is further bound to a cytokine. In one aspect, provided is a method of producing a CAR expressing cell. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or a vector system encodes the CAR. In some embodiments of any aspect as disclosed herein, the cell is selected from a Hematopoietic stem cell (HSC), an induced pluripotent stem cell (iPSCs), or an immune cell.

In some embodiments of any aspect as disclosed herein, the cell population comprises, or consists essentially of, or yet further consists of one or more of the following: an HSC, an iPSC, or an immune cell.

In some embodiments of any aspect as disclosed herein, the immune cell is selected from T-cells, B cells, NK cells, dendritic cells, myeloid cells, monocytes, or macrophages. In some embodiments of any aspect as disclosed herein, the immune cell is derived from an HSC or an iPSC.

In some embodiments of any aspect as disclosed herein, the method further comprises selecting or enriching the cell comprising the polynucleotide or the vector or a vector system as disclosed herein. Additionally or alternatively, the method further comprises selecting or enriching the cell expressing the CAR. In some embodiments, the method further comprises culturing the cell under a suitable condition, such as in a suitable culture medium, under a suitable culture temperature, with a suitable oxygen supply, or any combination thereof. In some embodiments, the method further comprises preserving the cell.

### Pharmaceutical Compositions

Additional aspects of the disclosure relate to compositions comprising, or alternatively consisting essentially of, or yet further consisting of, a carrier and one or more of the products—e.g., a cell population as disclosed herein, a CAR, an isolated cell comprising a CAR, a polypeptide, a polynucleotide, an isolated nucleic acid, a vector, a vector system, a cell, a cell population, or an isolated cell containing the CAR or the bispecific antibody or both as disclosed herein or nucleic acids encoding such—described in the embodiments disclosed herein. In some embodiments, the carrier is a pharmaceutically acceptable carrier. In further aspects, the composition may additionally comprise an immunoregulatory molecule.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for local or systemic administration, e.g., oral, intravenous, intracranial, topical, enteral, or parenteral administration. In certain embodiments, the compositions of the present disclosure are formulated for intravenous administration.

### Methods

In one aspect, provided is a method of inhibiting the growth of a cancer cell expressing PSCA or a tissue comprising the cancer cell. The method comprises, or consists essentially of, or yet further consists of contacting the cancer cell or the tissue with an isolated cell as disclosed herein or a cell population as disclosed herein. In some embodiments, the contacting is in vitro, or ex vivo, or in vivo. In some embodiments, the contacting is in vivo and the isolated cells are autologous or allogeneic to a subject being treated. In some embodiments, the contacting is in vivo and the isolated cells are allogenic to a subject being treated. When practiced in vitro, the method can be used to test for personalized therapy or alternatively, assay for combination therapies. When practiced in vivo, the method can be practiced in an animal to treat the animal, or alternatively, as an animal model to test personalized therapies or combination therapies. Methods to determine if the cell growth in vitro or in vivo are described herein and known in the art, e.g., by monitoring tumor growth by scanning technology in vivo. Alternatively, reduction in tumor burden, longer overall survival, longer progression free survival, longer time to tumor recurrence can also be monitored for determining the effectiveness of the therapy.

In some embodiments, the method further comprises contacting the cancer cell or the tissue with an effective amount of a therapy that upregulates the expression PSCA on the cancer cell.

In one embodiment, the contacting is in vivo and the isolated cells are autologous or allogeneic to a subject being treated. In another embodiment, the contacting is in vivo and the isolated cells are allogenic to a subject being treated.

In a further aspect, the method further comprises a therapy practiced prior to, concurrently or after the administration of the cells as described herein. Non-limiting examples include for example, cytoreductive therapy or the administration of a second cancer agent, e.g., gemcibabine or aldoxorubicin.

In some embodiments, the second anti-cancer therapy comprises administering an antibody that recognizes and binds to a suicide gene product, such as RQR8 (in some embodiments, an anti-RQR8 antibody is an anti-CD34 antibody or an anti-CD20 antibody), after the administration of the cells, thereby eliminating the suicide-gene-product-expressing cells. In further embodiments, the anti-CD20 antibody is selected from one or more of the following: rituximab, ocrelizumab, ofatumumab, binutuzumab, ibritumomab, or iodine i 131 tositumomab. In yet further embodiments, the administration of the antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells.

In some embodiments, the cytoreductive therapy comprises, or consists essentially of, or yet further consists of one or more of the following: a chemotherapy, a cryotherapy, a hyperthermia, a targeted therapy, or a radiation therapy.

In a further aspect, the method further comprises determining if the cancer cell expresses PSCA using methods know in the art, and then administering to a subject with the PSCA expressing tumor or cancer cell the compositions as described herein.

In another aspect, provided is a method for treating a cancer that expresses PSCA in a subject in need thereof. The method comprises, or consists essentially of, or yet further consists of administering the subject an isolated cell as disclosed herein or a cell population as disclosed herein to the subject, thereby treating the cancer. Methods to determine if the cancer has been treated include, for example, monitoring tumor growth by scanning technology in vivo. Alternatively, reduction in tumor burden, longer overall survival, longer progression free survival, longer time to tumor recurrence can also be monitored for determining the effectiveness of the therapy.

In yet another aspect, provided is a method for treating a solid tumor or cancer in a patient in need thereof, comprising, or alternatively consisting essentially of, or yet further consisting of administering a population of autologous or allogenic human NK cells transduced by a vector comprising, or alternatively consisting essentially of, or yet further consisting of a nucleic acid molecule disclosed herein, and wherein the solid tumor or cancer comprises cells expressing PSCA.

In yet another aspect, provided is a method of reducing or eliminating PSCA-positive cells in a subject comprising administering a population of autologous or allogeneic human NK cells transduced by a vector comprising a nucleic acid molecule disclosed herein.

In some embodiments, the subject has been selected for the therapy by determining expression of PSCA in a sample isolated from the subject and the sample comprise a cell expressing PSCA. In some embodiments, the sample is suspect of comprising or comprises a cancer cell. In further embodiments, the sample is a cancer or tumor biopsy. In some embodiments, the expression is determined by contacting the sample with an anti-PSCA antibody or an antigen binding fragment thereof in vitro, or ex vivo, or in vivo and detecting binding between the sample and the antibody or antigen binding fragment thereof. In further embodiments, the antibody or antigen binding fragment comprises a detectable marker.

In some embodiments, the method further comprises monitoring the therapy by isolating a sample from the subject and determining expression of PSCA in the sample isolated from the subject.

In some embodiments, the method further comprises determining expression of PSCA in a sample isolated from the subject prior to administration of the cell or the cell population.

In some embodiments, the method further comprises administering to the subject a cytoreductive therapy, or a therapy that upregulates the expression of PSCA, or both.

In a further aspect, the method further comprises a therapy practiced prior to, concurrently or after the administration of the cells as described herein. Non-limiting examples include for example, cytoreductive therapy or the administration of a second cancer agent, e.g., gemcibabine or aldoxorubicin.

In some embodiments, the cytoreductive therapy comprises, or consists essentially of, or yet further consists of one or more of the following: a chemotherapy, a cryotherapy, a hyperthermia, a targeted therapy, or a radiation therapy.

In some embodiments, the administration is applied to the subject as a first line therapy, or a second line therapy, or third line therapy, or a fourth line therapy.

In some embodiments, the second anti-cancer therapy comprises administering an antibody that recognizes and binds to a suicide gene product, such as RQR8 (in some embodiments, an anti-RQR8 antibody is an anti-CD34 antibody or an anti-CD20 antibody), after the administration of the cells, thereby eliminating the suicide-gene-product-expressing cells. In further embodiments, the anti-CD20 antibody is selected from one or more of the following: rituximab, ocrelizumab, ofatumumab, binutuzumab, ibritumomab, or iodine i 131 tositumomab. In yet further embodiments, the administration of the antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells.

In some embodiments, the cell (such as the isolated or engineered cell) is autologous or allogeneic to the subject in need. In some embodiments, the cell (such as the isolated or engineered cell) is allogenic to the subject in need.

In some embodiments of a method as disclosed herein, the administration is applied to the subject as a first line therapy, or a second line therapy, or third line therapy, or a fourth line therapy.

In some embodiments of any aspect as disclosed herein, the subject is a mammal, a canine, a feline, an equine, a murine, or a human patient.

In some embodiments where tEGFR is used as the suicide gene product or the detectable marker or both, the method further comprises administering an antibody that recognizes and binds tEGFR (an anti-tEGFR antibody) after the administrating the cell, thereby eliminating tEGFR expressing cells. In some embodiments, the administration of the anti-tEGFR antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells. Suitable anti-tEGFR antibody may be selected from one or more of the following: cetuximab, necitumumab, or panitumumab.

In some embodiments where the suicide gene product or detectable marker or both comprises, or consists essentially of, or yet further consists of RQR8, the method further comprises administering an antibody that recognizes and binds RQR8 (i.e., an anti-RQR8 antibody, such as an anti-CD20 antibody or an anti-CD34 antibody or both) after the administrating the cell, thereby eliminating RQR8 expressing cells. In some embodiments, the anti-RQR8 antibody recognizes and binds a CD34 epitope in the RQR8. Additionally or alternatively, the anti-RQR8 antibody recognizes and binds a CD20 epitope in the RQR8. In some embodiments, the anti-CD20 antibody is selected from one or more of the following: rituximab, ocrelizumab, ofatumumab, binutuzumab, ibritumomab, or iodine i 131 tositumomab. In some embodiments, the administration of the anti-RQR8 antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells. Suitable anti-RQR8 antibody may be selected from one or more of the following: rituximab, ofatumumab, ocrelizumab, obinutuzumab, ibritumomab, or tositumomab.

In some embodiments where the suicide gene product or detectable marker or both comprises, or consists essentially of, or yet further consists of RQR8, the method further comprises purifying the cells by selecting RQR8 expressing cells. In some embodiments, an anti-RQR8 antibody is used to select the RQR8 expressing cells. In further embodiments, a system available to one of skill in the art, such as CliniMACS CD34 Reagent System is used to select the RQR expressing cells.

In some embodiments, the cancer cell is a primary cancer cell or a metastatic cancer cell. In some embodiments, the cancer cell is from a carcinoma, an adenocarcinoma, gallbladder adenocarcinoma and transitional cell carcinoma a sarcoma, a myeloma, a leukemia, or a lymphoma. In some embodiments, the cancer is selected from a pancreatic cancer, a prostate cancer, urinary bladder cancer, a cervical cancer, an esophageal cancer, or a gastric cancer.

In some embodiments, the administering step may be repeated for once, twice, three times, four times, five times, six times, seven times, eight times, nine times, ten times or more. In further embodiments, two administrations are about 1 day apart, about 2 days apart, about 3 days apart, about 4 days apart, about 5 days apart, about 6 days apart, about 1 week apart, about 10 days apart, about 2 weeks apart, about 3 weeks apart, about 4 weeks apart, about 1 month apart, about 2 months apart, about 3 months apart, about 4 months apart, about 5 months apart, about 6 months apart, about 7 months apart, about 8 months apart, about 9 months apart, about 10 months a part, about 11 months apart, about 1 year apart, about 1.5 years apart, about 2 years apart, about 3 years apart, about 5 years apart, about 10 years apart or longer.

Administration of the cells or compositions can be performed in one dose, continuously or intermittently throughout the course of treatment and an effective amount to achieve the desired therapeutic benefit is provided. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cell are administered to the host or subject using methods known in the art and described, for example, in WO2012079000A1. This administration of the cells or compositions of the disclosure can be done to generate an animal model of the desired disease, disorder, or condition for experimental and screening assays.

### Combination Therapies

The compositions as described herein can be administered as first line, second line, third line, fourth line, or other therapy and can be combined with another anti-cancer therapy, such as cytoreductive interventions. They can be administered sequentially or concurrently as determined by the treating physician.

In some embodiments, the composition or method as disclosed herein can be combined with therapies that may upregulate the expression of a tumor or other antigen to which the CAR binds. In some embodiments, some clinical drugs can increase targeted antigens.

In some embodiments, the composition or method as disclosed herein can be combined with another anti-cancer therapy, such as cytoreductive interventions, or surgical removal of the cancer or tumor. In some embodiments, the cytoreductive therapy comprises, or alternatively consists essentially of, or yet consists of one or more of chemotherapy, cryotherapy, hyperthermia, targeted therapy, immunotherapy, or radiation therapy.

In some embodiments, the immunotherapy regulates immune checkpoints. In further embodiments, the immunotherapy comprises, or consists essentially of, or yet further consists of an immune checkpoint inhibitor, such as an Cytotoxic T-Lymphocyte Associated Protein 4 (CTLA4) inhibitor, or a Programmed Cell Death 1 (PD-1) inhibitor, or a Programmed Death Ligand 1 (PD-L1) inhibitor. In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KNO35 (envafolimab)).

In some embodiments, the compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

### Kits

As set forth herein, the present disclosure provides methods for producing and administering CAR cells. In one particular aspect, the present disclosure provides kits for performing these methods as well as instructions for carrying out the methods of the present disclosure such as collecting cells or tissues, performing the screen/transduction/etc., analyzing the results, or any combination thereof.

In one aspect, the kit comprises, or alternatively consists essentially of, or yet further consists of, any one or more of: a polypeptide as disclosed herein, a CAR as disclosed herein, a polynucleotide as disclosed herein, a vector as disclosed herein, a vector system as disclosed herein, a cell as disclosed herein, such as isolated allogenic cells, preferably T cells or NK cells, a cell population as disclosed herein, a composition as disclosed herein, an isolated complex as disclosed herein, or an optional instruction for use in a method as disclosed herein, for example, on the procuring of autologous cells from a patient. Such a kit may also comprise, or alternatively consist essentially of, or yet further comprise media and other reagents appropriate for the transduction, selection, activation, expansion or any combination thereof of CAR expressing cells, such as those disclosed herein.

In one aspect the kit comprises, or alternatively consists essentially of, or yet further consists of, an isolated CAR expressing cell or population thereof. In some embodiments, the cells of this kit may require activation or expansion or both prior to administration to a subject in need thereof. In further embodiments, the kit may further comprise, or consist essentially of, media and reagents, such as those covered in the disclosure above, to activate or expand or both activate and expand the isolated CAR expressing cell. In some embodiments, the cell is to be used for a CAR therapy. In further embodiments, the kit comprises instructions on the administration of the isolated cell to a patient in need of a CAR therapy.

The kits of this disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXAMPLES

The disclosed PSCA CARs and peptides and their use is further described in the following examples, which do not limit the scope the claims.

Materials and Methods

The following materials and methods were used in the Examples set forth herein.

Cell Lines

The PSCA(+) Canpan-1 and PSCA(−) PANC-1 cell lines were cultured in RPMI-1640 (Lonza) containing 10% fetal bovine serum (FBS, Hyclone) (complete RPMI) or in Dulbecco's Modified Eagles Medium (DMEM, Life Technologies) containing 10% FBS, 1X AA, 25 mM HEPES (Irvine Scientific), and 2 mM L-Glutamine (Fisher Scientific) (complete DMEM). All cells were cultured at 37° C. with 5% C02.

DNA Constructs, Lentivirus Production, and Retrovirus Production

PSCA CAR Construction

PSCA CAR containing anti-PSCA scFv-hinge-CD28-CD3z-2A fragment and the IL-15-2A-truncated EGFR (tEGFR) fragment were directly synthesized and then cloned into a retroviral or a lentiviral vector. The inserted sequences and orientations were confirmed by sequencing.

Retrovirus Production

Retrovirus carrying PSCA CAR was produced by co-transfecting an envelope plasmid, RD 114, RD114-TR, or VSVG in GP2-293T cells using lipofectamine, polyethylenimine (PEI), or CaHpo4. Retrovirus were collected 48 hours post transfection, filtered through 0.45 μm filter, and frozen at −80° C. in aliquot.

PSCA CAR NK Production and Assays

Generation of PSCA CAR NK

Primary NK cells were enriched from cord blood using a negative selection kit following manufacturer's instructions and expanded with IL-21 and 41BBL K562 feeders prior to retroviral infection. Expanded NK cells were infected with retrovirus. CAR NK transduction rate was determined by the expression of EGFR (Biolegend, #352906) by FACS analysis 72 hours post viral infection.

Real Time Cell Analysis (RTCA)

The in vitro function of CAR NK was evaluated by RTCA assay (ACEA Bioscience, xCELLigence RTCA MP). Briefly, target cell media was added to each well of 96 well E-Plates (ACEA Biosciences) and the background impedance was measured prior to adding target cells. Target cells were seeded at a density of 8,000 cells/well and incubated in the xCELLigence RTCA MP instrument at 37-degree incubator for 24 hours. CAR NK cells were then added to the target cells in a 1:3 E/T ratio and data were collected every 15-minute intervals for 72 hours. The normalized cell index for the target cells was analyzed using xIMT software (ACEA Biosciences).

Pancreatic Metastasis Model

Female NSG mice that were 8 weeks old were engrafted with pancreatic liver metastatic tumor cells (0.2 million of CAPAN1/mouse) expressing luciferase by i.p. injection. Tumor engraftment was confirmed by IVIS imaging prior to CAR NK treatment. CAR NK cells were directly recovered from liquid nitrogen and given to the mice by IP (2 million/mouse) and IV (2 million/mouse) injection. CAR NK were given in three cycles and each cycle contained 4 doses and performed in two weeks. Total 12 doses were given in 6 weeks.

IVIS Imaging

D-luciferin substrate (Biosynth, #L8220) was dissolved by PBS and given to the CAPAN1-Luc engrafted mice by IP injection (150 mg/kg). Mice were anesthetized by 2% isoflurane and oxygen (1 L/min) in an image chamber then subject to image acquisition. Luminescent image was captured by Lago-X (Spectral Instruments Imaging) following manufacturer's instructions. Luminescence was quantified by Aura Imaging Software (Version 2.2.1.1).

Generation of PSCA CAR NK Cells

The PSCA CAR constructs for NK cells were composed with two different anti-PSCA scFv domains (FIG. 2).

In some cases, the CAR constructs have a signal peptide, an anti-PSCA which can comprise a variable light chain and a variable heavy chain joined by a flexible linker in either VL-linker-VH or VH-linker-VL conformation, a spacer or hinge from IgG1 or IgG4, a transmembrane domain, a CD28 or 2B4 costimulatory domain, CD3ξ optionally, a codon optimized IL-15 (soluble or membrane bound and both with and without being complexed with IL-15a), and a truncated human epidermal growth factor receptor truncated EGFR or CD19 or LNGFR as a marker (FIG. 2). The CAR construct can also use other costimulatory domains such as 41BB, 2B4, other spacer domains and transmembrane domains such as CD28, NKG2D, CD8 hinge, and CD4 transmembrane domain. The IL-15 domain, which can comprise IL-15 alone or IL-15 and IL-15Rα connected by a linker, can follow the transmembrane domain. For CAR constructs for NK cells, the transmembrane domain can be the CD28 or NKG2D transmembrane domain and the costimulatory domain can be the CD28 or 2B4 costimulatory domain. All experiments were performed using primary human NK cells.

Validation that PSCA CAR NK Cells Selectively Target PSCA-Positive Cells

To determine whether PSCA CAR NK cells demonstrate selective activity against PSCA-positive cancer and noncancerous cells, the PSCA CAR NK cells were co-cultured with either PSCA-positive cells or PSCA-negative cells.

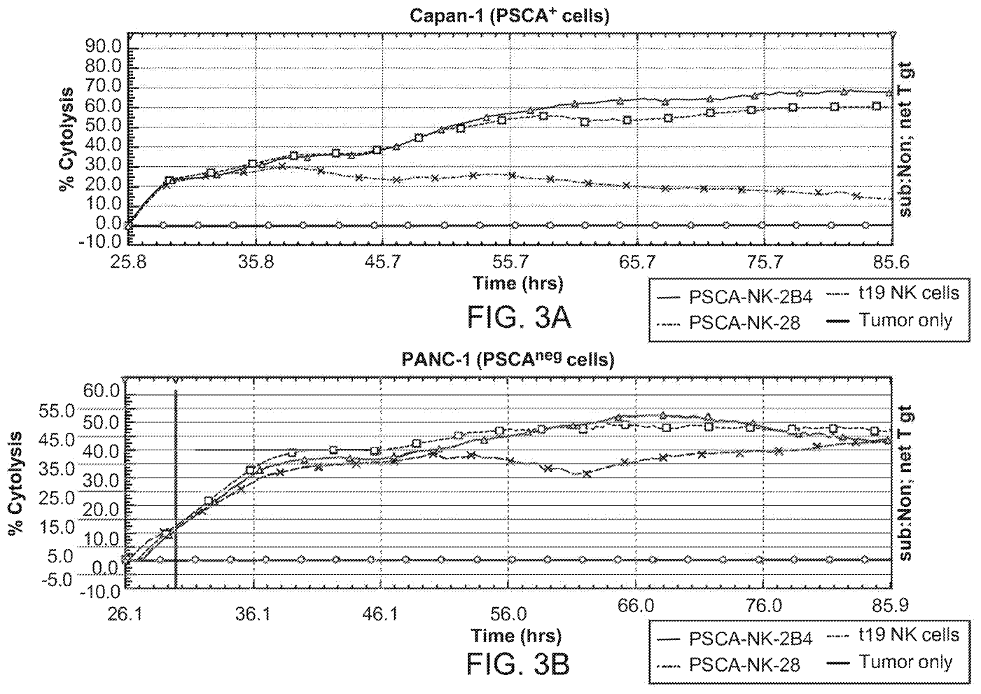

As shown in FIGS. 3A-3B, PSCA-CAR NK cells with either the CD28 or 2B4 costimulatory domain (PSCA-NK-28 and PSCA-NK-2B4, respectively) lyse pancreatic PSCA⁺ tumor cells in an antigen-specific manner compared to NK cells expressing truncated CD19 without PSCA-CAR (t19 NK cells) in vitro. Data were collected using the xCELLigence Real-Time Cell Analysis (RTCA). Both of the PSCA-CAR NK cells lyse PSCA(+) Canpan-1 pancreatic cancer cells (FIG. 3A). Importantly, there is no CAR activity of PSCA-CAR NK cells against PSCA(−) PANC-1 cell line. The t19 NK cells and the condition with only tumor cells served as a control.

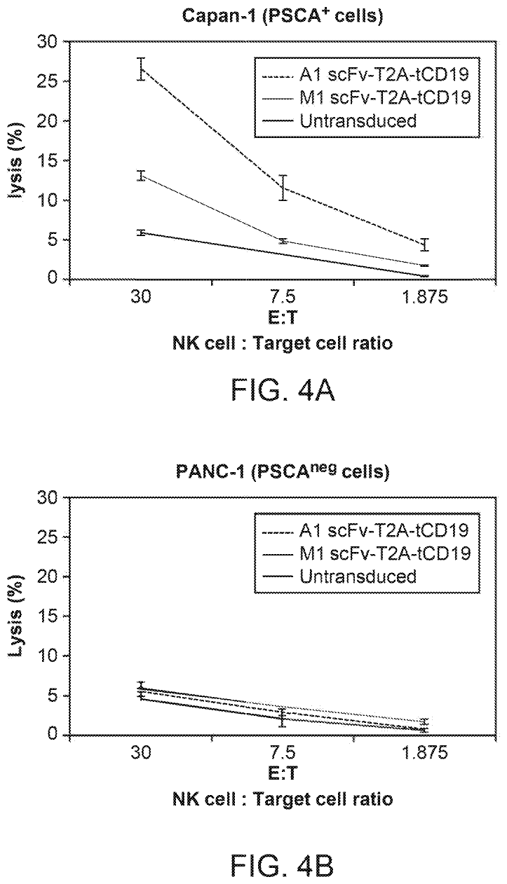

Two different codon-optimized PSCA scFv sequences were used in tumor killing assays. A1 scFv comprises SEQ ID NOs: connected with a flexible linker. M1 scFv comprises SEQ ID NOs: connected with a flexible linker. As shown in FIGS. 4A-4B, both scFv constructs of PSCA CAR NK cells were co-cultured with either PSCA⁺ CAPAN-1 tumor cells or PSCA^{neg} PANC-1 cells at the shown effector:target ratios (30:1, 7.5:1, and 1.875:1). Data were collected using a ⁵C release assay. Both the A1 scFv CAR NK cells and M1 scFv CAR NK cells effectively lysed PSCA⁺ CAPAN-1 tumor cells at all ratios (FIG. 4A). The PSCA^{neg} PANC-1 cells served as a control and show CAR-mediated cell lysis is antigen specific (FIG. 4B).

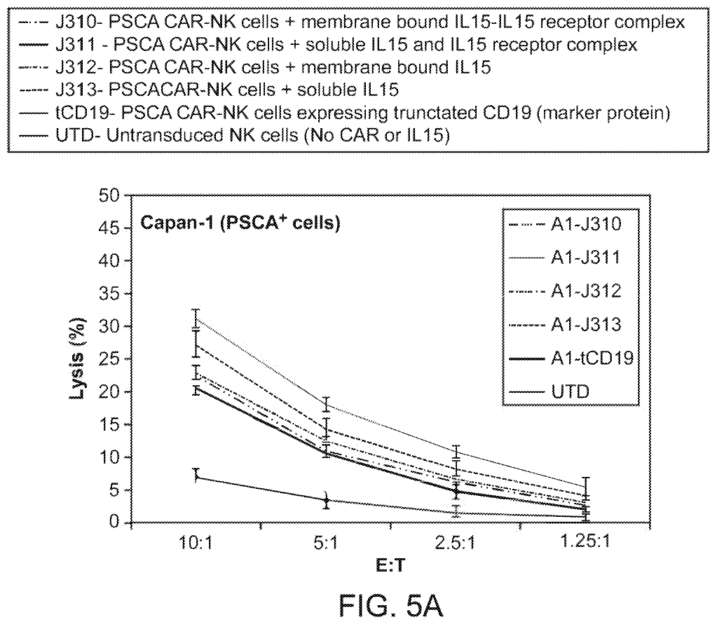

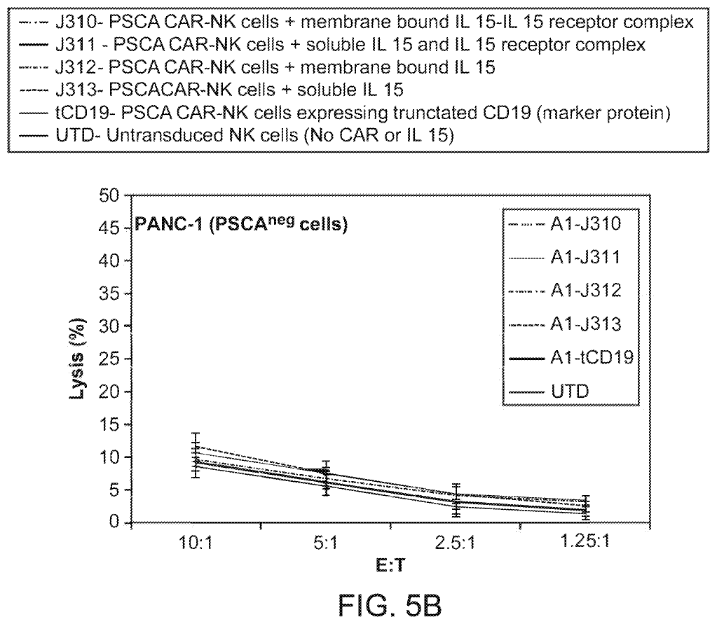

Cytotoxicity of PSCA CARs comprising 4 different types of IL-15 domains, including soluble (s)IL-15, membrane bound (mb)IL-15, sIL-15 complex IL-15Rα, and mbIL-15 complexed with IL-15a, were tested either PSCA⁺ CAPAN-1 tumor cells or PSCA^{neg} PANC-1 cells at the shown effector:target ratios (10:1, 5:1, 2.5:1, and 1.25:1). Data were collected using a ⁵¹C release assay. All A1 PSCA CAR NK cells effectively lysed PSCA⁺ CAPAN-1 tumor cells (FIG. 5A). PSCA CAR NK cells comprising an IL-15 domain lysed cells the tumor cells as effectively or, in some cases, more effectively than the PSCA CAR NK cells without the IL-15 domain (“A1-tCD19”). In particular, the PSCA CAR NK cells with soluble IL-15 and IL-15 complex had a surprising increase in antigen specific tumor cell lysis (FIG. 5A, “A1-J311” red) followed closely by PSCA CAR NK cells with soluble IL-15 (FIG. 5A, “A1-J313” purple). The PSCA^{neg} PANC-1 cells served as a control and show the CAR-mediated cell lysis is antigen specific (FIG. 5B).

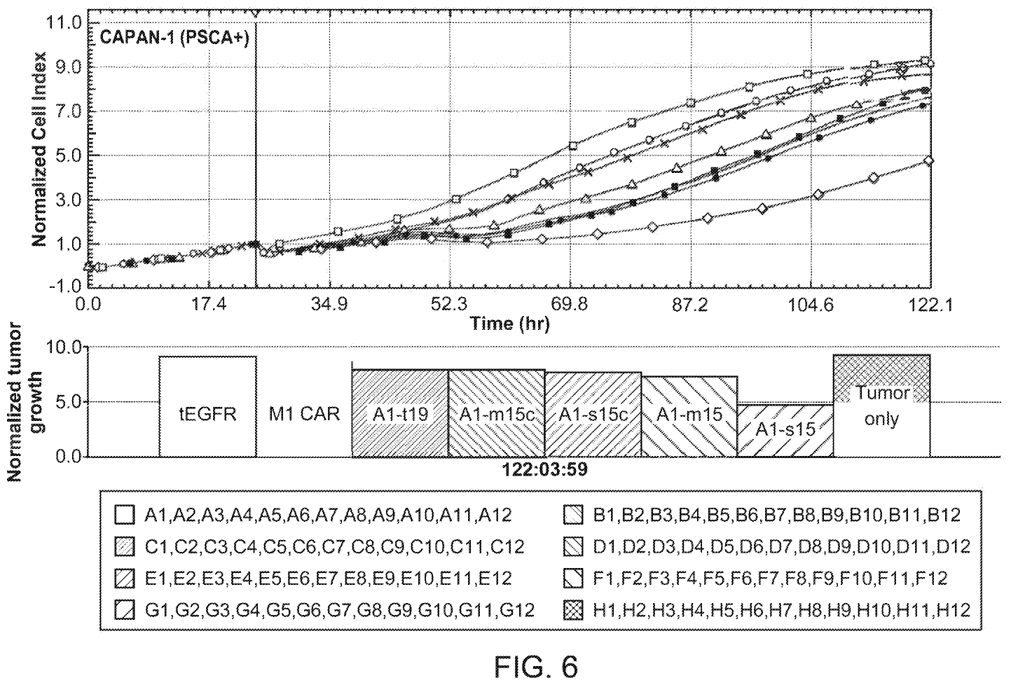

A shown in FIG. 6, the cytotoxicity of PSCA-CAR NK cells with 4 different types of IL-15 against PSCA(+) Canpan-1 pancreatic cancer cells was confirmed and quantified using xCELLigence Real-Time Cell Analysis (RTCA). The top panel shows the normalized tumor growth index over time (in hours following the initiation of co-culturing the PSCA⁺ CAPAN-1 tumor cells with the PSCA CAR NK cells). The bottom panel shows quantitative data at one time point of around 25 hours (see the vertical solid line in the top panel) after setting up the co-culture of tumor cells and CAR NK cells. Again, all PSCA CAR NK cells led to a decrease in tumor growth compared to untreated (“tumor only”) and the tEGFR NK cells without the anti-PSCA domain. The A1 PSCA CAR NK cells with soluble IL-15 (A1-si5) reduced tumor growth the most, followed by A1 PSCA CAR NK cells with membrane bound IL-15 (A1-m15), A1 PSCA CAR NK cells with soluble IL-15 complex IL-15Rα (A1-s15c), A1 PSCA CAR NK cells with membrane bound IL-15 complex IL-15Rα (A1-m15c), and A1 PSCA CAR NK cells without an IL-15 domain (A1-t19).

Validation that PSCA CAR NK Cells Delivered In Vivo in a Mouse Model Exhibit Potent Anti-Tumor Activity

To evaluate in vivo efficacy of PSCA CAR NK cells to selectively target PSCA-positive cells in the mouse model, PSCA CAR NK cells were delivered and tumor size was evaluated over time.

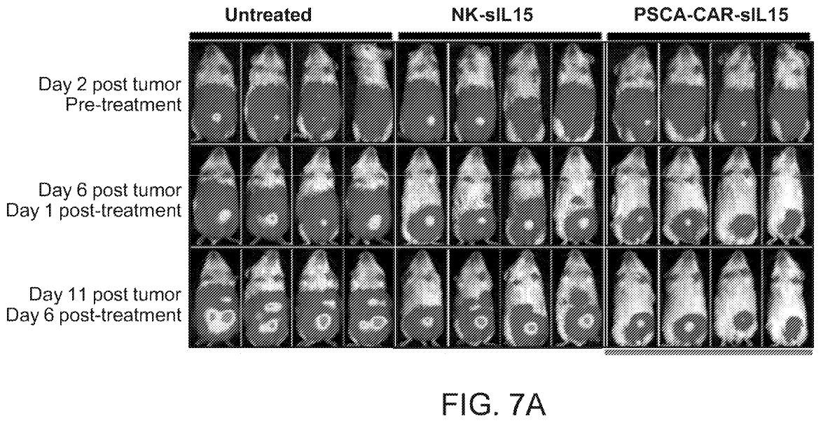

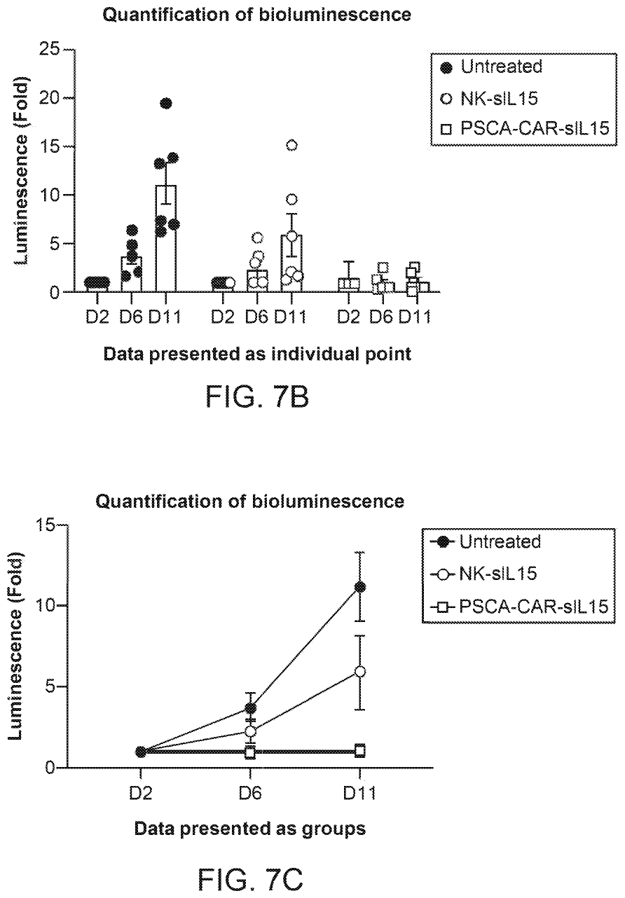

As FIGS. 7A-7C show, the manufactured frozen, off-the-shelf primary human PSCA-CAR-sIL-15 NK cells show in vivo efficacy and eradicate tumor cells more effectively when compared to the untreated group and also better than the group with sIL-15-transduced NK cells without a PSCA CAR in the animal model with mice engrafted with PSCA(+) Capan-1 cells. The human PSCA-CAR-sIL-15 NK cells treatment significantly eliminated tumor engrafts.

NK Cells Expressing PSCA CAR and Soluble IL-15 Target PSCA Expressing Pancreatic Cells Lines

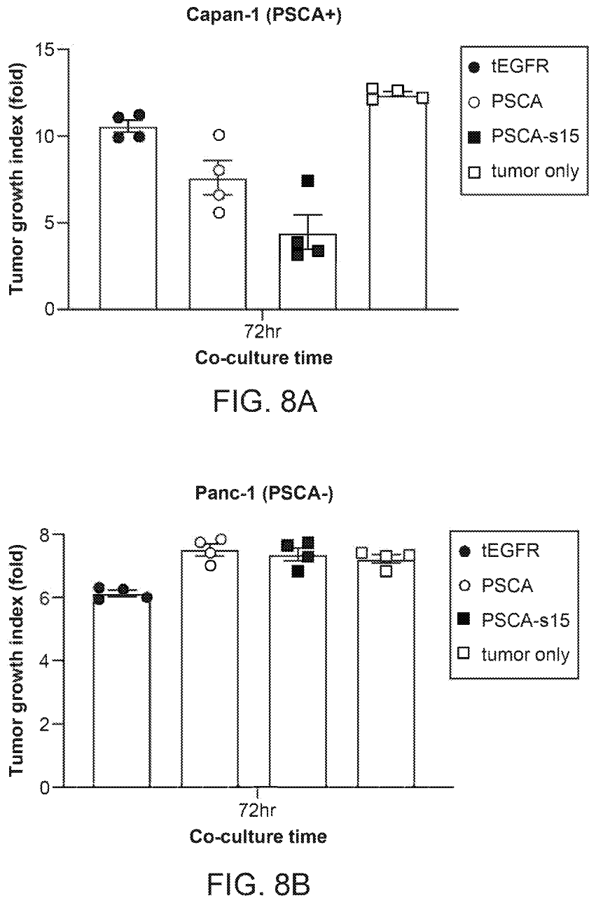

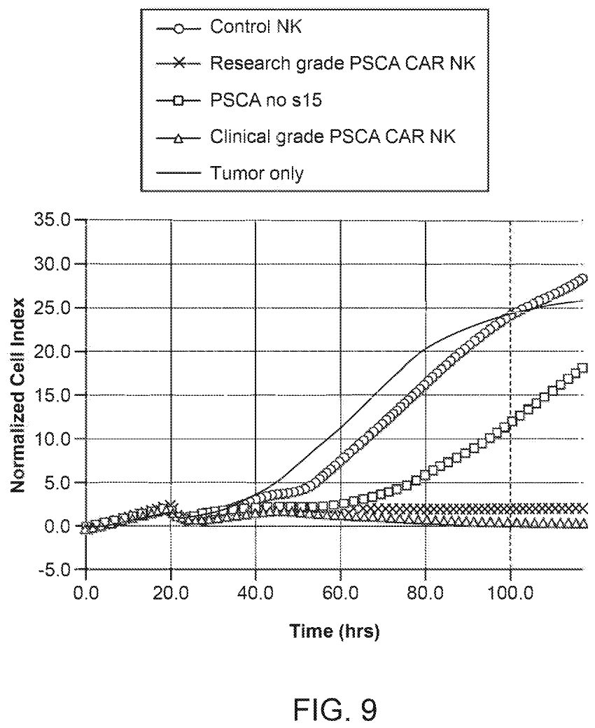

PSCA CAR NK cells-expressing soluble IL-15 were tested for cytotoxicity against two different pancreatic cell lines. FIG. 8A shows the impact on Capan-1 (PSCA⁺) cells and FIG. 8B shows the impact on Panc-1 (PSCA−) cells. Cytotoxicity was tested after co-culture of NK cells with pancreatic cells for a duration of 72 hrs at an E:T ratio of 1:3.

Research and clinical PSCA CAR NK vector expressing soluble IL-15 were tested for cytotoxicity against Capan-1 (PSCA⁺) cells. Non-human DNA sequences were removed from the research grade PSCA CAR—soluble IL-15 vector to create the clinical grade PSCA CAR—soluble IL-15 vector. Cytotoxicity was tested after co-culture of indicated NK cells or PSCA CAR—soluble NK cells with pancreatic cells for a duration of 72 hrs at an E:T ratio of 1:3. Cell growth was analyzed by the xCELLigence Real-Time Cell Analysis (RTCA).

Anti-Tumor Effect of PSCA CAR—Soluble IL15 NK Cells

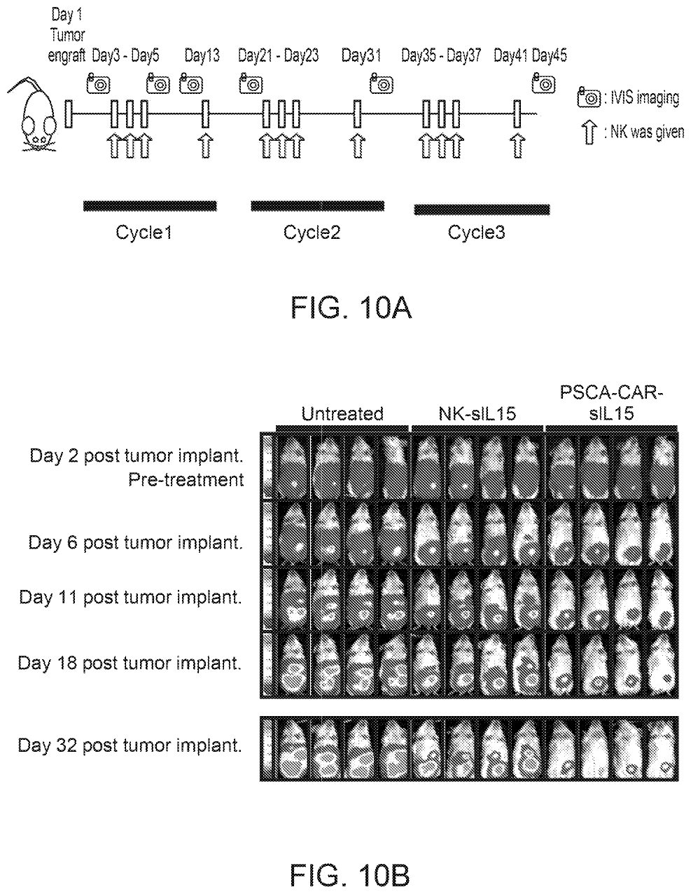

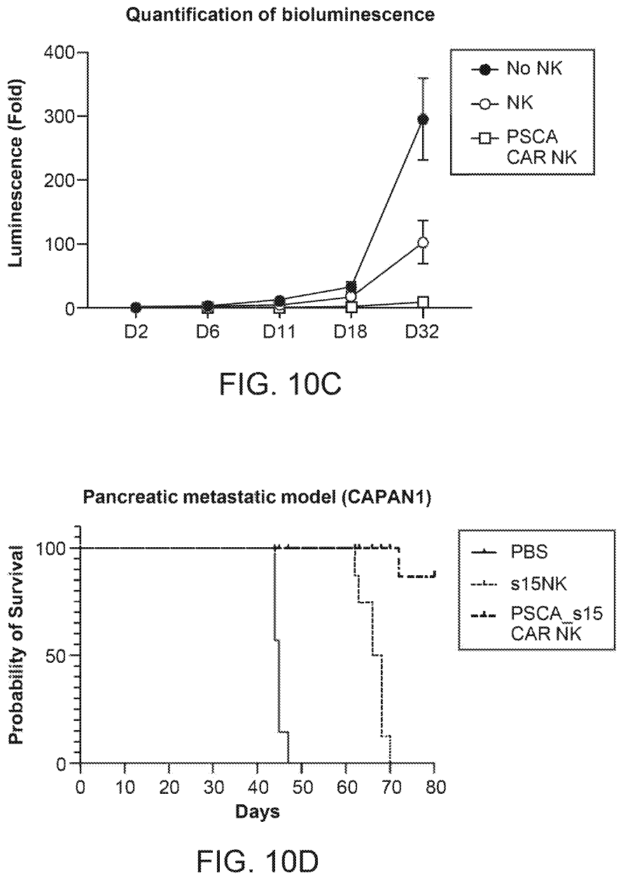

As schematically depicted in FIG. 10A, NSG mice were intraperitoneally engrafted with 0.2×10⁹ CAPAN1 cells expressing luciferase (luc) then received three cycles of treatment with NK cells expressing soluble IL-15 or PSCA CAR and soluble IL-15. Each cycle included four injections each composed of 2×10⁹ cells administered intraperitoneally and 2×10⁹ administered intravenously. FIG. 10B is IVIS imaging of mice that were engrafted with CAPAN1_luc and treated with or without the indicated NK or CAR NK cells expressing soluble IL-15. Mice were imaged prior- and post-NK treatment. FIG. 10C shows a quantification of bioluminescence of pancreatic cancer cells. FIG. 10D shows survival analysis of mice engrafted with CAPAN1_luc and treated with or without indicated NK cells or CAR NK cells expressing soluble IL-15.

PSCA CAR—Soluble IL15 NK Cells are Effective in Combination with Anticancer Drugs

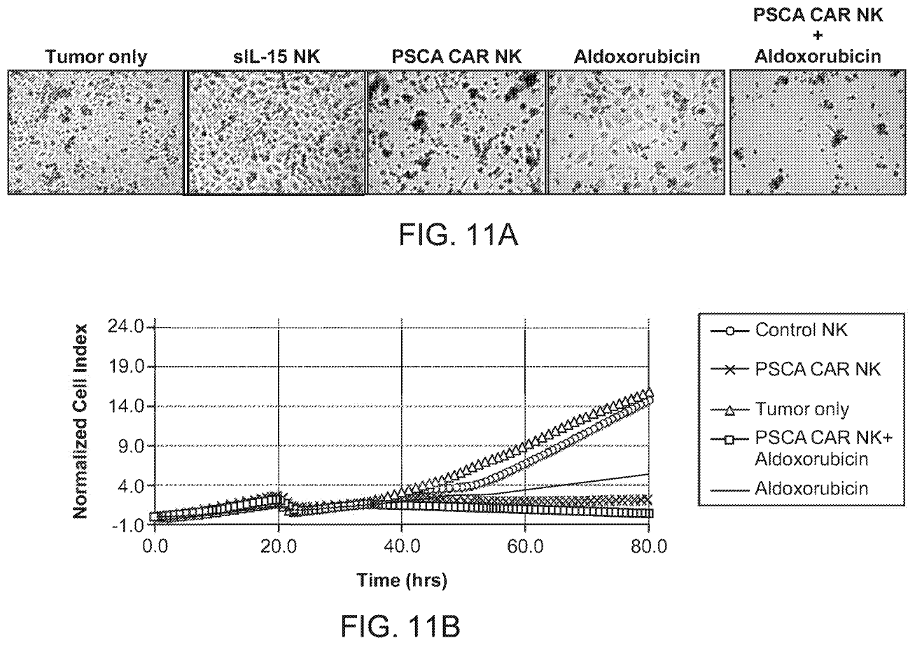

FIG. 11A is a series of representative images of 48 hr post co-culture of the pancreatic cancer cell line CAPAN1 with PSCA CAR—soluble IL-15 NK cells (Effector/Target=1:3, PSCA CAR NK) or aldoxorubicin (0.3 μM) or in combination. FIG. 11B depicts an analysis of normalized tumor growth index of CAPAN1 treated with PSCA CAR—soluble IL-15 NK cells, aldoxorubicin (0.3 μM), or in combination, analyzed by the xCELLigence Real-Time Cell Analysis (RTCA). NK cells expressing soluble IL-15 served as control.

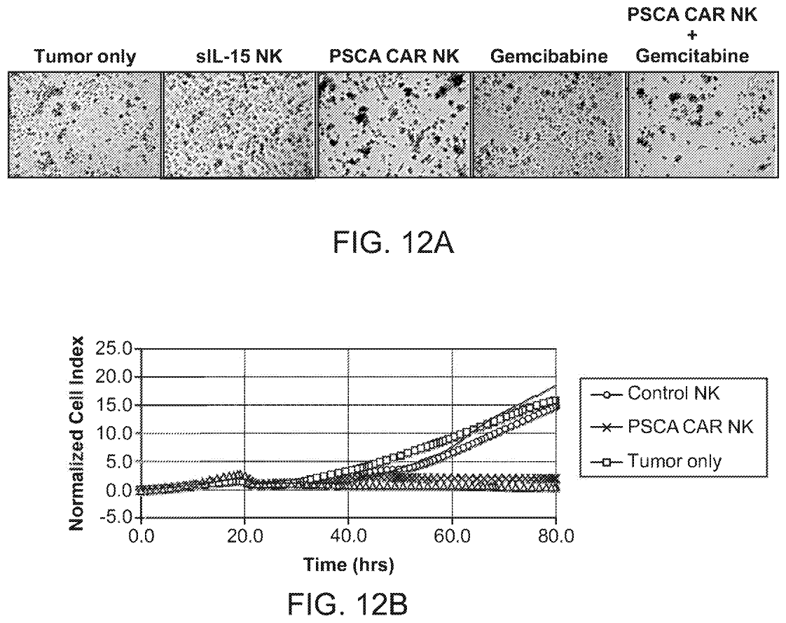

FIG. 12A is a series of representative images of 48 hr post co-culture of the pancreatic cancer cell line CAPAN1 with PSCA CAR NK—soluble IL-15 cells expressing (Effector/Target=1:3, PSCA CAR NK) or gemcibabine (0.3 μM) or in combination. FIG. 12B depicts an analysis of normalized tumor growth index of CAPAN1 treated with PSCA CAR—soluble IL-15 NK cells, aldoxorubicin (0.3 μM), or in combination, analyzed by the xCELLigence Real-Time Cell Analysis (RTCA). NK cells expressing soluble IL-15 served as control.

Cryopreservation does not Impact PSCA CAR NK Cell Function

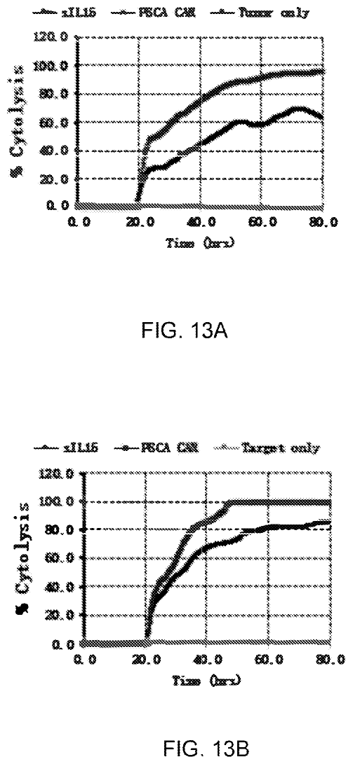

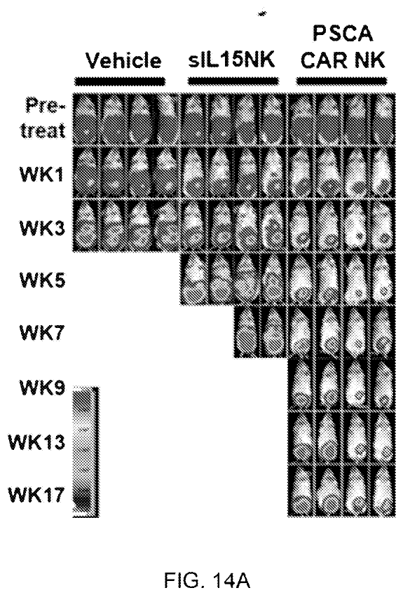

FIG. 13A and FIG. 13B depict the results of in vitro tumor cell lysis assays using freshly prepared and previously frozen, respectively, NK cells expressing soluble IL-15 only (sIL-15) or soluble IL-15 and a PSCA CAR (PSCA CAR). As can be seen, freezing did not significantly impact the activity of the PSCA CAR.

Anti-Tumor Effect of PSCA CAR—Soluble IL15 NK Cells

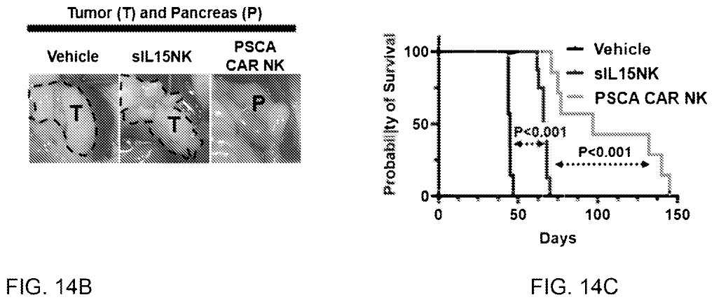

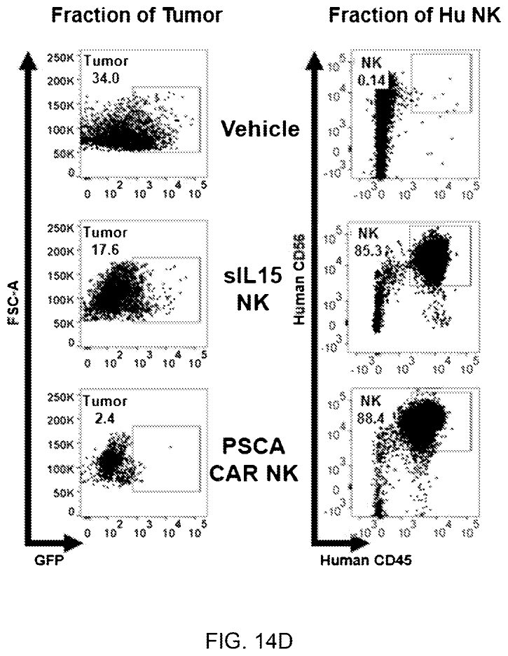

NSG mice were intraperitoneally engrafted with 0.2×10⁶ CAPAN1 cells expressing luciferase (luc) then received three cycles of treatment with saline, NK cells expressing soluble IL-15 or PSCA CAR NK cells expressing soluble IL-15. Each cycle included four injections with each injection composed of 2×10⁶ cells administered intraperitoneally and 2×10⁶ administered intravenously. FIG. 14A is IVIS imaging of mice that were engrafted with CAPAN1-luc and treated with saline or with the indicated NK cells expressing soluble IL-15 or CAR NK cells expressing soluble IL-15. The treatment periods were Days 4-18, Days 19-33 and Days 34-48. Mice were imaged prior- and post-NK treatment. FIG. 14B is images of the pancreas of saline treated, sIL-15 NK cell treated or PSCA CAR/sIL-15 treated mice. FIG. 14C shows survival analysis of mice engrafted with CAPAN1_luc and treated with NK cells expressing sIL-15 or PSCA CAR NK cells expressing sIL-15. Treatment with the PSCA CAR/sIL-15 NK cells significantly suppressed tumor growth, protected the pancreas and liver from metastatic spread, and significantly prolonged survival of the mice over either control arm (saline or sIL-15 NK cells) of the study.

FACS analysis was performed at Day 45 to assess the presence of tumor cells and NK cells cells isolated from the pancreas. As can be seen in FIG. 14D, PSCA CAR/sIL-15 NK treatment resulted in the near-absence of tumor cells and the persistence of PSCA CAR NK cells.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A nucleic acid molecule comprising:
(i) a first nucleotide molecule encoding a chimeric antigen receptor (CAR), and
(ii) a second nucleotide molecule encoding an IL-15 domain,
wherein the CAR comprises: a single chain variable fragment (scFv) targeting prostate stem cell antigen (PSCA), a spacer, a transmembrane domain, a co-stimulatory domain, and a CD3 ξ signaling domain.

2. The nucleic acid molecule of claim 1, wherein the scFv comprises a heavy chain (HC) complementarity-determining region (CDR) 1 (CDRH1) comprising DYYI (aa 31 to aa 34 of SEQ ID NO: 33), an HC CDR 2 (CDRH2) comprising WIDPENGDTEFVPKFQG (aa 50 to aa 66 of SEQ ID NO: 33), and an HC CDR 3 (CDRH3) comprising GGF (aa 99 to aa 101 of SEQ ID NO: 33).

3. The nucleic acid molecule of claim 1, wherein the scFv comprises a light chain (LC) complementarity-determining region (CDR) 1 (CDRL1) comprising SASSSVRFIH (aa 24 to aa 33 of SEQ ID NO: 32), an LC CDR 2 (CDRL2) comprising DTSKLAS (aa 49 to aa 55 of SEQ ID NO: 32), and an LC CDR 3 (CDRL3) comprising QQWGSSPFT (aa 88 to aa 96 of SEQ ID NO: 32).

4. The nucleic acid molecule of claim 1, wherein the scFv comprises a CDRH1 comprising DYYI (aa 31 to aa 34 of SEQ ID NO: 33), a CDRH2 comprising WIDPENGDTEFVPKFQG (aa 50 to aa 66 of SEQ ID NO: 33), a CDRH3 comprising GGF (aa 99 to aa 101 of SEQ ID NO: 33), a CDRL1 comprising SASSSVRFIH (aa 24 to aa 33 of SEQ ID NO: 32), a CDRL2 comprising DTSKLAS (aa 49 to aa 55 of SEQ ID NO: 32), and a CDRL3 comprising QQWGSSPFT (aa 88 to aa 96 of SEQ ID NO: 32).

5. The nucleic acid molecule of claim 1, wherein the scFv comprises a light chain variable region of SEQ ID NO: 32 or an equivalent thereof, and a heavy chain variable region of SEQ ID NO: 33 or an equivalent thereof.

6. The nucleic acid molecule of claim 1, wherein the scFv comprises the amino acid sequence of SEQ ID NOs: 32 and 33, or an equivalent of each thereof.

7. The nucleic acid molecule of claim 1, wherein the scFv comprises a CDRH1 comprising SYSMS (aa 31 to aa 35 of SEQ ID NO: 35), a CDRH2 comprising YINDSGGSTFYPDTVKG (aa 50 to aa 66 of SEQ ID NO: 35), and a CDRH3 comprising RMYYGNSHWHFDV (aa 99 to aa 111 of SEQ ID NO: 35).

8. The nucleic acid molecule of claim 1, wherein the scFv comprises a CDRL1 comprising GTSQDINNYLN (aa 24 to aa 34 of SEQ ID NO: 34), a CDRL2 comprising YTSRLHS (aa 50 to aa 56 of SEQ ID NO: 34), and a CDRL3 comprising QQSKTLPWT (aa 89 to aa 97 of SEQ ID NO: 34).

9. The nucleic acid molecule of claim 1, wherein the scFv comprises a CDRH1 comprising SYSMS (aa 31 to aa 35 of SEQ ID NO: 35), a CDRH2 comprising YINDSGGSTFYPDTVKG (aa 50 to aa 66 of SEQ ID NO: 35), a CDRH3 comprising RMYYGNSHWHFDV (aa 99 to aa 111 of SEQ ID NO: 35), a CDRL1 comprising GTSQDINNYLN (aa 24 to aa 34 of SEQ ID NO: 34), a CDRL2 comprising YTSRLHS (aa 50 to aa 56 of SEQ ID NO: 34), and a CDRL3 comprising QQSKTLPWT (aa 89 to aa 97 of SEQ ID NO: 34).

10. The nucleic acid molecule of claim 1, wherein the scFv comprises a light chain variable region of SEQ ID NO: 34 or an equivalent thereof, and a heavy chain variable region of SEQ ID NO: 35 or an equivalent thereof.

11. The nucleic acid molecule of claim 1, wherein the scFv comprises the amino acid sequence of SEQ ID NOs: 1, 40, 41 or 42, or an equivalent of each thereof.

12. The nucleic acid molecule of claim 1,
wherein the transmembrane domain comprises any one of: a CD4 transmembrane domain, a CD8 transmembrane domain, a CD28 transmembrane domain, or a NKG2D transmembrane domain, and
wherein the costimulatory domain comprises any one or more of a CD28, a 4-1BB, or a 2B4 costimulatory domain, and
wherein a linker of 3 to 15 amino acids is located between the costimulatory domain and the CD3 ξ signaling domain.

13. (canceled)

14. The nucleic acid molecule of claim 1,
wherein the transmembrane domain comprises any one of SEQ ID NOs: 13-20, 85, 86, or 93-95, or an equivalent of each thereof, and
wherein the costimulatory domain comprises the amino acid sequence of any one or more of SEQ ID NOs: 22-25 or 66, or an equivalent of each thereof, and
wherein the CD3ξ signaling domain comprises the amino acid sequence of SEQ ID NO: 21, or an equivalent thereof, and
wherein the spacer comprises any one of SEQ ID NOs: 2-12 or 44, or an equivalent of each thereof.

15.-19. (canceled)

20. The nucleic acid molecule of claim 1, wherein the CAR further comprises a signal sequence or a linker located between the VL and the VH encoding nucleic acid molecules and wherein the PSCA antigen binding domain comprises VL-linker-VH or VH-linker-VL, and wherein the signal sequence is any one of a human GM-CSF receptor alpha signal sequence, an IgGk signal peptide, an IgG2 signal peptide, or an IL-2 signal peptide, or wherein the signal sequence comprises any one of SEQ ID NOs: 29-31, 36, or 106-108, or an equivalent of each thereof.

21. (canceled)

22. (canceled)

23. The nucleic acid molecule claim 20, wherein the CAR comprises the amino acid sequence of any one of SEQ ID NOs: 1, 40, 41 or 42, or an equivalent of each thereof, or a variant of each thereof having 1-5 amino acid modifications.

24. The nucleic acid molecule of claim 1, wherein the IL-15 domain comprises a soluble human IL-15 or a fusion protein comprising soluble IL-15 and a function portion of IL-15Rα, or the IL-15 domain comprises SEQ ID NO: 43 and SEQ ID NO: 72 connected by a linker, or the IL-15 domain comprises SEQ ID NO: 70 and SEQ NO: 72, connected by a linker, or an equivalent of each thereof, or
wherein the IL-15 domain comprises a membrane-bound IL-15 wherein the IL-15 domain comprises SEQ ID NOs: 43, 70, or 72, or an equivalent of each thereof.

25.-27. (canceled)

28. The nucleic acid molecule of claim 1,
wherein the second polypeptide further comprises a human IL-15Rα, or
wherein the IL-15Rα comprises SEQ ID NO: 72, or an equivalent thereof, or
wherein the second polypeptide comprises a fusion protein comprising a soluble IL-15 and an IL-15Rα, optionally connected by a linker, or
wherein the nucleic acid molecule further comprises a first ribosomal skip sequence or a fourth nucleotide sequence encoding a self-cleaving peptide located between the first nucleotide sequence and the second nucleotide sequence.

29.-31. (canceled)

32. The nucleic acid molecule of claim 1, wherein the nucleic acid molecule further comprises a third nucleotide sequence encoding a detectable marker or a suicide gene product or both, and
wherein the third nucleotide sequence encodes any one of tEGFR, tCD19, or LNGFR domain, or
wherein the third nucleotide sequence encodes SEQ ID NO: 45 or an equivalent thereof.

33. (canceled)

34. (canceled)

35. The nucleic acid molecule of claim 32, comprising from 5′ to 3′, any one of the following:
(1) the first nucleotide sequence, the first ribosomal skip sequence or the third nucleotide sequence encoding the self-cleaving peptide, the second nucleotide sequence, a second ribosomal skip sequence or a fifth nucleotide sequence encoding a self-cleaving peptide located, and the fourth nucleotide sequence;
(2) the first nucleotide sequence, the first ribosomal skip sequence or the third nucleotide sequence encoding the self-cleaving peptide, the fourth nucleotide sequence, the second ribosomal skip sequence or the fifth nucleotide sequence encoding the self-cleaving peptide located, and the second nucleotide sequence;
(3) the second nucleotide sequence, the first ribosomal skip sequence or the third nucleotide sequence encoding the self-cleaving peptide, the first nucleotide sequence, the second ribosomal skip sequence or the fifth nucleotide sequence encoding the self-cleaving peptide located, and the fourth nucleotide sequence;
(4) the second nucleotide sequence, the first ribosomal skip sequence or the third nucleotide sequence encoding the self-cleaving peptide, the fourth nucleotide sequence, the second ribosomal skip sequence or the fifth nucleotide sequence encoding the self-cleaving peptide located, and the first nucleotide sequence;
(5) the fourth nucleotide sequence, the first ribosomal skip sequence or the third nucleotide sequence encoding the self-cleaving peptide, the first nucleotide sequence, the second ribosomal skip sequence or the fifth nucleotide sequence encoding the self-cleaving peptide located, and the second nucleotide sequence; or
(6) the fourth nucleotide sequence, the first ribosomal skip sequence or the third nucleotide sequence encoding the self-cleaving peptide, the second nucleotide sequence, the second ribosomal skip sequence or the fifth nucleotide sequence encoding the self-cleaving peptide located, and the first nucleotide sequence.

36.-37. (canceled)

38. The nucleic acid molecule of claim 1, further comprising any one or more of: SEQ ID NOs: 46 or 47, or an equivalent of each thereof.

39. (canceled)

40. A vector comprising the nucleic acid molecule of claim 1.

41.-46. (canceled)

47. A cell comprising one or more of the following: a nucleic acid molecule of claim 1.

48.-58. (canceled)

59. A method of inhibiting the growth of a cancer cell expressing PCSA or a tissue comprising the cancer cell, comprising contacting the cancer cell or the tissue with a cell of claim 47.

60. (canceled)

61. (canceled)

62. A method for treating a cancer that expresses PSCA in a subject in need thereof, comprising administering the subject a cell of claim 47, thereby treating the cancer.

63. (canceled)

64. (canceled)

65. A method for treating a solid tumor or cancer in a patient comprising administering a population of autologous or allogeneic human NK cells transduced by a vector comprising the nucleic acid molecule of claim 1, wherein the solid tumor or cancer comprises cells expressing PSCA.

66. A method of reducing or eliminating PSCA-positive cells in a subject comprising administering a population of autologous or allogeneic human NK cells transduced by a vector comprising the nucleic acid molecule of any one of claim 1.

67.-81. (canceled)

82. A human NK cells transduced by a vector comprising the nucleic acid molecule of claim 1, wherein the human NK cells are stable after one, two, or three freeze-thaw cycles.

83. (canceled)

84. (canceled)


SEQUENCE LISTING (112 sequences)

SEQ ID NO: 1 - protein, 237 aa, Artificial Sequence
  Description of Artificial Sequence: Synthetic polypeptide
  PSCA scFv (A1 LH)
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGGSGGGGSGGGGSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 2 - protein, 10 aa, Artificial Sequence
  Description of Artificial Sequence: Synthetic peptide
  linker
GGGSSGGGSG

SEQ ID NO: 3 - protein, 12 aa, Artificial Sequence
  Description of Artificial Sequence: Synthetic peptide
  IgG4 hinge (S228P)
ESKYGPPCPPCP

SEQ ID NO: 4 - protein, 12 aa, Homo sapiens
  IgG4 hinge
ESKYGPPCPSCP

SEQ ID NO: 5 - protein, 22 aa, Homo sapiens
  IgG4 hinge (S228P)+ linker
ESKYGPPCPPCPGGGSSGGGSG

SEQ ID NO: 6 - protein, 39 aa, Homo sapiens
  CD28 hinge
IEVMYPPPYLDNEKSNGTIIHVKGKHLCPSPLFPGPSKP

SEQ ID NO: 7 - protein, 48 aa, Homo sapiens
  CD8 hinge-48aa
AKPTTTPAPRPPTPAPTIASQPLSLRPEACRPAAGGAVHTRGLDFACD

SEQ ID NO: 8 - protein, 45 aa, Homo sapiens
  CD8 hinge-45aa
TTTPAPRPPTPAPTIASQPLSLRPEACRPAAGGAVHTRGLDFACD

SEQ ID NO: 9 - protein, 129 aa, Artificial Sequence
  Description of Artificial Sequence: Synthetic polypeptide
  IgG4(HL-CH3)
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  Description of Artificial Sequence: Synthetic polypeptide
  IgG4(L235E,N297Q)
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  Description of Artificial Sequence: Synthetic polypeptide
  IgG4(S228P,L235E,N297Q)
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  Description of Artificial Sequence: Synthetic polypeptide
  IgG4(CH3)
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
  CD3z transmembrane domain
LCYLLDGILFIYGVILTALFL

SEQ ID NO: 14 - protein, 27 aa, Homo sapiens
  CD28 transmembrane domain
FWVLVVVGGVLACYSLLVTVAFIIFWV

SEQ ID NO: 15 - protein, 28 aa, Homo sapiens
  CD28(m) transmembrane domain
MFWVLVVVGGVLACYSLLVTVAFIIFWV

SEQ ID NO: 16 - protein, 22 aa, Homo sapiens
  CD4 transmembrane domain
MALIVLGGVAGLLLFIGLGIFF

SEQ ID NO: 17 - protein, 21 aa, Homo sapiens
  CD8 alpha transmembrane domain
IYIWAPLAGTCGVLLLSLVIT

SEQ ID NO: 18 - protein, 23 aa, Homo sapiens
  CD8tm2 transmembrane domain
IYIWAPLAGTCGVLLLSLVITLY

SEQ ID NO: 19 - protein, 24 aa, Homo sapiens
  CD8tm3 transmembrane domain
IYIWAPLAGTCGVLLLSLVITLYC

SEQ ID NO: 20 - protein, 27 aa, Homo sapiens
  41BB transmembrane domain
IISFFLALTSTALLFLLFFLTLRFSVV

SEQ ID NO: 21 - protein, 112 aa, Homo sapiens
  CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
  CD28 co-signaling domain
RSKRSRLLHSDYMNMTPRRPGPTRKHYQPYAPPRDFAAYRS

SEQ ID NO: 23 - protein, 41 aa, Homo sapiens
  CD28gg* costimulatory domain
RSKRSRGGHSDYMNMTPRRPGPTRKHYQPYAPPRDFAAYRS

SEQ ID NO: 24 - protein, 42 aa, Homo sapiens
  4-1BB costimulatory signaling region
KRGRKKLLYIFKQPFMRPVQTTQEEDGCSCRFPEEEEGGCEL

SEQ ID NO: 25 - protein, 42 aa, Homo sapiens
  OX40 costimulatory domain
ALYLLRRDQRLPPDAHKPPGGGSFRTPIQEEQADAHSTLAKI

SEQ ID NO: 26 - protein, 323 aa, Artificial Sequence
  Description of Artificial Sequence: Synthetic polypeptide
  truncated CD19t
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Artificial Sequence
  Description of Artificial Sequence: Synthetic peptide
  ribosomal skip sequence
LEGGGEGRGSLLTCGDVEENPGPR

SEQ ID NO: 28 - protein, 354 aa, Artificial Sequence
  Description of Artificial Sequence: Synthetic polypeptide
  truncated EGFR
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 20 aa, Artificial Sequence
  Description of Artificial Sequence: Synthetic peptide
  IgGk signal peptide
METDTLLLWVLLLWVPGSTG

SEQ ID NO: 30 - protein, 18 aa, Artificial Sequence
  Description of Artificial Sequence: Synthetic peptide
  IgG2 signal peptide
MGWSSIILFLVATATGVH

SEQ ID NO: 31 - protein, 20 aa, Artificial Sequence
  Description of Artificial Sequence: Synthetic peptide
  IL-2 signal peptide
MYRMQLLSCIALSLALVTNS

SEQ ID NO: 32 - protein, 110 aa, Artificial Sequence
  Description of Artificial Sequence: Synthetic polypeptide
  PSCA scFv (A1 VL)
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTS

SEQ ID NO: 33 - protein, 112 aa, Artificial Sequence
  Description of Artificial Sequence: Synthetic polypeptide
  PSCA scFv (A1 VH)
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 34 - protein, 107 aa, Artificial Sequence
  Description of Artificial Sequence: Synthetic polypeptide
  PSCA scFv (M1 VL)
DIQMTQSPSSLSASVGDRVTITCGTSQDINNYLNWYQQKPGKVPKLLIYYTSRLHSGVPSRFSGSGSGTDFTLTISSLQPEDVATYYCQQSKTLPWTFGGGTQLTVL

SEQ ID NO: 35 - protein, 122 aa, Artificial Sequence
  Description of Artificial Sequence: Synthetic polypeptide
  PSCA scFv (M1 VH)
QVQLVESGGGLVKPGGSLRLSCAASGFTFSSYSMSWIRQAPGKGLEWVSYINDSGGSTFYPDTVKGRFTISRDNAKNSLYLQMNSLRAEDTAVYYCARRMYYGNSHWHFDVWGQGTTVTVSS

SEQ ID NO: 36 - protein, 22 aa, Homo sapiens
  GM-CSF receptor alpha signal sequence
MLLLVTSLLLCELPHPAFLLIP

SEQ ID NO: 37 - protein, 15 aa, Artificial Sequence
  Description of Artificial Sequence: Synthetic peptide
  linker
GGGGSGGGGSGGGGS

SEQ ID NO: 38 - protein, 4 aa, Artificial Sequence
  Description of Artificial Sequence: Synthetic peptide
  linker
GGGS

SEQ ID NO: 39 - protein, 5 aa, Artificial Sequence
  Description of Artificial Sequence: Synthetic peptide
  linker
GGGGS

SEQ ID NO: 40 - protein, 237 aa, Artificial Sequence
  Description of Artificial Sequence: Synthetic polypeptide
  PSCA scFv (A1 HL)
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGGGGSGGGGSGGGGSDIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTS

SEQ ID NO: 41 - protein, 244 aa, Artificial Sequence
  Description of Artificial Sequence: Synthetic polypeptide
  PSCA scFv (M1 LH)
DIQMTQSPSSLSASVGDRVTITCGTSQDINNYLNWYQQKPGKVPKLLIYYTSRLHSGVPSRFSGSGSGTDFTLTISSLQPEDVATYYCQQSKTLPWTFGGGTQLTVLGGGGSGGGGSGGGGSQVQLVESGGGLVKPGGSLRLSCAASGFTFSSYSMSWIRQAPGKGLEWVSYINDSGGSTFYPDTVKGRFTISRDNAKNSLYLQMNSLRAEDTAVYYCARRMYYGNSHWHFDVWGQGTTVTVSS

SEQ ID NO: 42 - protein, 244 aa, Artificial Sequence
  Description of Artificial Sequence: Synthetic polypeptide
  PSCA scFv (M1 HL)
QVQLVESGGGLVKPGGSLRLSCAASGFTFSSYSMSWIRQAPGKGLEWVSYINDSGGSTFYPDTVKGRFTISRDNAKNSLYLQMNSLRAEDTAVYYCARRMYYGNSHWHFDVWGQGTTVTVSSGGGGSGGGGSGGGGSDIQMTQSPSSLSASVGDRVTITCGTSQDINNYLNWYQQKPGKVPKLLIYYTSRLHSGVPSRFSGSGSGTDFTLTISSLQPEDVATYYCQQSKTLPWTFGGGTQLTVL

SEQ ID NO: 43 - protein, 133 aa, Homo sapiens
  soluble IL-15 sequence
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 44 - protein, 16 aa, Unknown
  Description of Unknown: IgG1 Hinge
LEPKSCDKTHTCPPCP

SEQ ID NO: 45 - protein, 357 aa, Artificial Sequence
  Description of Artificial Sequence: Synthetic polypeptide
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 46 - protein, 18 aa, Thosea asigna virus
  T2A
EGRGSLLTCGDVEENPGP

SEQ ID NO: 47 - protein, 22 aa, Artificial Sequence
  Description of Artificial Sequence: Synthetic peptide
  P2A
GSGATNFSLLKQAGDVEENPGP

SEQ ID NO: 48-64 - skipped (17 entries)

SEQ ID NO: 65 - protein, 21 aa, Homo sapiens
  NKG2D transmembrane domain
PFFFCCFIAVAMGIRFIIMVA

SEQ ID NO: 66 - protein, 120 aa, Homo sapiens
  2B4 costimulatory signaling region
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 67-69 - skipped (3 entries)

SEQ ID NO: 70 - protein, 334 aa, Artificial Sequence
  Description of Artificial Sequence: Synthetic polypeptide
  IL-15 domain
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 71 - protein, 26 aa, Artificial Sequence
  Description of Artificial Sequence: Synthetic peptide
  linker
SGGGSGGGGSGGGGSGGGGSGGGSLQ

SEQ ID NO: 72 - protein, 175 aa, Homo sapiens
  IL-15Ra
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 73 - skipped

SEQ ID NO: 74 - protein, 23 aa, Unknown
  Description of Unknown: Transmembrane domain
VAISTSTVLLCGLSAVSLLACYL

SEQ ID NO: 75 - protein, 332 aa, Artificial Sequence
  Description of Artificial Sequence: Synthetic polypeptide
  IL-15 domain
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 76 - protein, 354 aa, Artificial Sequence
  Description of Artificial Sequence: Synthetic polypeptide
  IL-15 domain
VAISTSTVLLCGLSAVSLLACYLITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 77 - protein, 156 aa, Unknown
  Description of Unknown: IL-15 transmembrane domain
VAISTSTVLLCGLSAVSLLACYLGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 78 - protein, 282 aa, Artificial Sequence
  Description of Artificial Sequence: Synthetic polypeptide
  truncated LNGFR
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNK

SEQ ID NO: 79 - protein, 129 aa, Artificial Sequence; identical to SEQ ID NO: 9
  Description of Artificial Sequence: Synthetic polypeptide
  spacer sequence

SEQ ID NO: 80 - protein, 65 aa, Unknown
  Description of Unknown: IL15 receptor
ITCPPPMSVEHADIWVKSYSLYSRERYICNSGFKRKAGTSSLTECVLNKATNVAHWTTPSLKCIR

SEQ ID NO: 81 - protein, 24 aa, Artificial Sequence
  Description of Artificial Sequence: Synthetic peptide
  linker
SGGGSGGGGSGGGGSGGGGSGGGS

SEQ ID NO: 82 - protein, 556 aa, Homo sapiens [sequence omitted]

SEQ ID NO: 83 - protein, 557 aa, Homo sapiens
  CD19 isoform 2
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 84 - DNA, 48 bp, Unknown
  Description of Unknown: IgG1 heavy chain hinge
ctcgagcccaaatcttgtgacaaaactcacacatgcccaccgtgcccg

SEQ ID NO: 85 - DNA, 81 bp, Unknown
  Description of Unknown: CD28 transmembrane region
ttttgggtgctggtggtggttggtggagtcctggcttgctatagcttgctagtaacagtggcctttattattttctgggtg

SEQ ID NO: 86 - DNA, 126 bp, Unknown
  Description of Unknown: 4-1BB co-stimulatory signaling region
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 87 - DNA, 123 bp, Unknown
  Description of Unknown: CD28 co-stimulatory signaling region
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 88 - DNA, 339 bp, Unknown
  Description of Unknown: CD3 zeta signaling region
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 89 - protein, 51 aa, Homo sapiens
  CD8 alpha hinge domain
PAKPTTTPAPRPPTPAPTIASQPLSLRPEACRPAAGGAVHTRGLDFACDIY

SEQ ID NO: 90 - protein, 49 aa, Mus muculus
  CD8 alpha hinge domain
KVNSTTTKPVLRTPSPVHPTGTSQPQRPEDCRPRGSVKGTGLDFACDIY

SEQ ID NO: 91 - protein, 51 aa, Unknown
  Description of Unknown: Cat CD8 alpha hinge domain
PVKPTTTPAPRPPTQAPITTSQRVSLRPGTCQPSAGSTVEASGLDLSCDIY

SEQ ID NO: 92 - protein, 220 aa, Homo sapiens
  CD28 Sequence
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 93 - protein, 21 aa, Mus muculus
  CD8 alpha transmembrane domain
IWAPLAGICVALLLSLIITLI

SEQ ID NO: 94 - protein, 21 aa, Rattus sp.
  CD8 alpha transmembrane domain
IWAPLAGICAVLLLSLVITLI

SEQ ID NO: 95 - protein, 68 aa, Homo sapiens
  CD28 transmembrane and cytoplasmic domain
FWVLVVVGGVLACYSLLVTVAFIIFWVRSKRSRLLHSDYMNMTPRRPGPTRKHYQPYAPPRDFAAYRS

SEQ ID NO: 96 - DNA, 105 bp, Unknown
  Description of Unknown: ICOS costimulatory signaling region
acaaaaaagaagtattcatccagtgtgcacgaccctaacggtgaatacatgttcatgagagcagtgaacacagccaaaaaatccagactcacagatgtgacccta

SEQ ID NO: 97 - DNA, 108 bp, Unknown
  Description of Unknown: OX40 costimulatory signaling region
agggaccagaggctgccccccgatgcccacaagccccctgggggaggcagtttccggacccccatccaagaggagcaggccgacgcccactccaccctggccaagatc

SEQ ID NO: 98 - protein, 20 aa, Unknown
  Description of Unknown: DAP10 costimulatory signaling region
RPRRSPAQDGKVYINMPGRG

SEQ ID NO: 99 - protein, 21 aa, Unknown
  Description of Unknown: DAP12 costimulatory signaling region
ESPYQELQGQRSDVYSDLNTQ

SEQ ID NO: 100 - protein, 114 aa, Unknown
  Description of Unknown: CD3zeta signaling domain
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 101 - protein, 108 aa, Unknown
  Description of Unknown: CD3zeta signaling domain
RVKFSRSADAPAYKQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 102 - protein, 108 aa, Unknown
  Description of Unknown: CD3zeta signaling domain
RVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 103 - protein, 114 aa, Unknown
  Description of Unknown: soluble IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 104 - protein, 162 aa, Unknown
  Description of Unknown: soluble IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 105 - protein, 61 aa, Artificial Sequence
  Description of Artificial Sequence: Synthetic polypeptide
  IL15RA sushi domain
  MOD_RES (3)..(3): P or A
  MOD_RES (5)..(5): M or V
  MOD_RES (13)..(13): W, R or Q
  MOD_RES (20)..(20): Y or H
  MOD_RES (26)..(26): I or V
  MOD_RES (51)..(51): V or A
  MOD_RES (53)..(53): V or A
CPXPXSVEHADIXVKSYSLXSRERYXCNSGFKRKAGTSSLTECVLNKATNXAXWTTPSLKC

SEQ ID NO: 106 - protein, 17 aa, Artificial Sequence
  Description of Artificial Sequence: Synthetic peptide
  signal peptide
MWLQSLLLLGTVACSIS

SEQ ID NO: 107 - protein, 29 aa, Artificial Sequence
  Description of Artificial Sequence: Synthetic peptide
  signal peptide
MRISKPHLRSISIQCYLCLLLNSHFLTEA

SEQ ID NO: 108 - protein, 24 aa, Artificial Sequence
  Description of Artificial Sequence: Synthetic peptide
  signal peptide
MRSSPGNMERIVICLMVIFLGTLV

SEQ ID NO: 109 - protein, 8 aa, Artificial Sequence
  Description of Artificial Sequence: Synthetic peptide
  2A peptide consensus motif
  MOD_RES (2)..(2): V or I
  MOD_RES (4)..(4): Any amino acid thought to be self-cleaving
DXEXNPGP

SEQ ID NO: 110 - protein, 75 aa, Artificial Sequence
  Description of Artificial Sequence: Synthetic polypeptide
  linker
  SITE (1)..(75): This sequence may encompass 1-15 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 111 - protein, 40 aa, Artificial Sequence
  Description of Artificial Sequence: Synthetic polypeptide
  linker
  SITE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 112 - protein, 50 aa, Artificial Sequence
  Description of Artificial Sequence: Synthetic polypeptide
  linker
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS